(19) United States
(12) Patent Application Publication
(10) Pub. No.: US 2024/0358293 A1
(43) Pub. Date: Oct. 31, 2024

(54) INCORPORATION OF SILVER SALTS WITHIN A TRANSCUTANEOUS BIOSENSOR FOR INFECTION CONTROL

(71) Applicant: Abbott Diabetes Care Inc., Alameda, CA (US)

(72) Inventors: Jonathan D. MCCANLESS, Oakland, CA (US); Benjamin J. FELDMAN, Berkeley, CA (US); Udo HOSS, San Ramon, CA (US)

(73) Assignee: Abbott Diabetes Care Inc., Alameda, CA (US)

(21) Appl. No.: 18/639,650

(22) Filed: Apr. 18, 2024

Related U.S. Application Data
(60) Provisional application No. 63/496,853, filed on Apr. 18, 2023.

(51) Int. Cl. A61B 5/1486; A61B 5/00; A61L 31/08; A61L 31/16

(52) U.S. Cl. CPC A61B 5/14865; A61B 5/6849; A61L 31/088; A61L 31/16; A61B 2562/125; A61L 2300/404

(57) ABSTRACT

The present disclosure provides analyte sensors comprising a sensor tail substrate having a lower portion and configured for insertion into a tissue; a first working electrode disposed on the lower portion of the sensor tail substrate; a first sensing layer disposed upon a surface of the first working electrode; and a membrane disposed over at least the sensing layer; wherein the membrane comprises 20% to 50% by weight of a silver salt. The present disclosure also provides methods of using such analyte sensors for detecting one or more analytes present in a biological sample and methods of manufacturing the analyte sensors.

### FIELD

The present disclosure provides analyte sensors comprising a sensor tail substrate having a lower portion and configured for insertion into a tissue; a first working electrode disposed on the lower portion of the sensor tail substrate; a first sensing layer disposed upon a surface of the first working electrode; and a membrane disposed over at least the sensing layer; wherein the membrane comprises 20% to 50% by weight of a silver salt. The present disclosure also provides methods of using such analyte sensors for detecting one or more analytes present in a biological sample and methods of manufacturing the analyte sensors.

### BACKGROUND

The detection of various analytes within an individual can sometimes be vital for monitoring the condition of their health and well-being. Deviation from normal analyte levels can be indicative of an underlying physiological condition, such as a metabolic condition or illness, or exposure to particular environmental factors or stimuli. Glucose levels, for example, can be particularly important to detect and monitor in diabetic individuals.

Analyte monitoring in an individual can occur periodically or continuously over a period of time. Periodic analyte monitoring can take place by withdrawing a sample of bodily fluid, such as blood, at set time intervals and analyzing ex vivo. Continuous analyte monitoring can be conducted using one or more sensors that remain implanted within a tissue of an individual, such as dermally, subcutaneously, or intravenously, through which analyses can take place in vivo. Implanted sensors can collect analyte data continuously, at planned intervals, or sporadically, depending on an individual's particular health needs and/or previously determined analyte levels.

Although the entirety of a sensor can be implanted within an individual (e.g., surgically), it is more common for at least the bioactive and communication path (e.g., flex circuit) portions of the sensor to be implanted internally (e.g., through a skin penetration), with one or more additional sensor components remaining external to the individual's body. In many instances, sensors suitable for measuring analyte levels in vivo can extend from a sensor housing that is designed to be worn “on-body” for extended periods of time, such as upon the skin. Such on-body analyte sensors can be especially desirable, since they often can be applied directly by a user, rather than relying on a medical professional to perform an invasive sensor implantation procedure.

Despite the desirability of on-body analyte sensors, their use can cause complications. When positioning an on-body analyte sensor onto the skin of a user, a needle or other introducer is used to puncture the skin and allow implantation of at least a portion of a sensor through the dermal region. Accordingly, a transdermal skin wound is created in order for the sensor to undergo positioning for analyte monitoring (i.e., the “insertion site,” including the actual wound and areas adjacent thereto), and at least an active portion of the sensor remains within the skin for the wear duration of the on-body analyte sensor. Both during localization and during wear, microorganism incursion into the wound at the sensor insertion site and along any length of the sensor, including the active area, can occur, such as by exposure to skin microorganisms and/or the external environment. The possibility of existing microorganisms near the insertion site and/or migration of microorganisms from adjacent areas, including the external environment, can create a rich environment for microorganism growth. Such growth can be harmful to the user and/or can lead to altered functioning of the analyte sensor itself, such as causing a shortened life of the sensor and/or providing erroneous or altered data or perceived sensitivity and/or response times. Therefore, a need exists to prevent or reduce microorganisms near and/or adjacent to the sensing layer of an analyte sensor that can result in erroneous measurements.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims, as well as from the appended drawings.

The present disclosure provides an analyte sensor comprising:
a sensor tail substrate having a lower portion and configured for insertion into a tissue;
a first working electrode disposed on the lower portion of the sensor tail substrate;
a sensing layer disposed upon a surface of the first working electrode; and
a membrane disposed over at least the sensing layer;
wherein the membrane comprises 20% to 50% by weight of a silver salt with a particle size of ≤60 μm.

The present disclosure provides an analyte sensor comprising:
a portion positionable into a tissue below the surface of the skin;
a first working electrode disposed on the portion positionable into a tissue;
a sensing layer disposed upon a surface of the first working electrode; and
a membrane disposed over at least the sensing layer;
wherein the membrane comprises 20% to 50% by weight of a silver salt with a maximum particle diameter of ≤60 μm.

In some embodiments, the silver salt in the membrane disposed over at least the sensing layer is uniformly distributed within the membrane.

In some embodiments, the silver salt in the membrane disposed over at least the sensing layer is selected from the group consisting of silver chloride, silver-silver chloride, silver iodide, and combinations thereof.

In some embodiments, the silver salt in the membrane disposed over at least the sensing layer is silver iodide.

In some embodiments, the silver salt in the membrane disposed over at least the sensing layer has a particle size of ≤53 μm.

In some embodiments, the silver salt in the membrane disposed over at least the sensing layer has a maximum particle diameter of ≤53 μm.

In some embodiments, the silver iodide in the membrane disposed over at least the sensing layer has a purity of at least 99%.

In some embodiments, the membrane disposed over at least the sensing layer comprises 30% to 40% by weight of silver iodide.

In some embodiments, the membrane disposed over at least the sensing layer comprises 35% by weight of silver iodide.

In some embodiments, the analyte sensor does not induce cytotoxicity as measured according to ISO 10993-5.

In some embodiments, the analyte sensor does not cause skin irritation or skin sensitization according to ISO 10993-10.

In some embodiments, the analyte sensor does not cause intradermal irritation according to ISO 10993-10.

In some embodiments, the analyte sensor does not cause systemic toxicity according to ISO 10993-11.

In some embodiments, the analyte sensor is non-hemolytic according to ISO 10993-4 and ASTM F 756.

In some embodiments, the analyte sensor does not cause intramuscular irritation according to ISO 10993-6.

In some embodiments, the analyte sensor is not a potential mutagen according to ISO 10993-3 and OECD 471.

The present disclosure provides a method of inhibiting microorganism growth in an area in and around an insertion site comprising inserting an analyte sensor in a subject in need thereof comprising:
a first portion positionable above a surface of the skin;
a second portion positionable below the surface of the skin, wherein the second portion is in contact with bodily fluid and configured to measure signals indicative of analyte levels in the bodily fluid;
a first working electrode disposed on the second portion;
a sensing layer disposed upon a surface of the first working electrode; and
a membrane disposed over at least the sensing layer;
wherein the membrane comprises 20% to 50% by weight of a silver salt with a

particle size of ≤60 μm, and wherein the analyte sensor does not induce cytotoxicity as measured according to ISO 10993-5.

The present disclosure also provides a method of inhibiting microorganism growth in an area in and around an insertion site comprising inserting an analyte sensor into a subject in need thereof, the analyte sensor comprising:
a first portion positionable above a surface of the skin;
a second portion positionable below the surface of the skin, wherein the second portion is in contact with bodily fluid and configured to measure signals indicative of analyte levels in the bodily fluid;
a first working electrode disposed on the second portion;
a sensing layer disposed upon a surface of the first working electrode; and
a membrane disposed over at least the sensing layer;
wherein the membrane comprises 20% to 50% by weight of a silver salt with a maximum particle diameter of ≤60 μm, and wherein the analyte sensor does not induce cytotoxicity as measured according to ISO 10993-5.

In some embodiments, the silver salt in the membrane disposed over at least the sensing layer is silver iodide.

In some embodiments, the silver iodide in the membrane disposed over at least the sensing layer has a particle size of ≤53 μm.

In some embodiments, the silver iodide in the membrane disposed over at least the sensing layer has a maximum particle diameter of ≤53 μm.

In some embodiments, the silver iodide in the membrane disposed over at least the sensing layer has a purity of at least 99%.

The present disclosure provides a method of preparing an analyte sensor overcoated with a membrane comprising:
(a) mixing a silver salt having a particle size of ≤60 μm and at least one membrane polymer to form a silver salt dispersion, wherein the dispersion comprises 20% to 50% by weight of the silver salt;
(b) dipping an analyte sensor into the silver salt dispersion to overcoat the analyte sensor with the silver salt dispersion;
(c) curing the analyte sensor; and
(d) baking the analyte sensor.

The present disclosure provides a method of preparing an analyte sensor overcoated with a membrane comprising:
(a) mixing a silver salt having a maximum particle diameter of ≤60 μm and at least one membrane polymer to form a silver salt dispersion, wherein the dispersion comprises 20% to 50% by weight of the silver salt;
(b) dipping an analyte sensor into the silver salt dispersion to overcoat the analyte sensor with the silver salt dispersion;
(c) curing the analyte sensor; and
(d) baking the analyte sensor.

In some embodiments, the silver salt mixed with at least one membrane polymer is silver iodide.

The present disclosure provides an analyte sensor comprising:
a sensor tail substrate having a lower portion and configured for insertion into a tissue;
a first working electrode disposed on the lower portion of the sensor tail substrate;
a sensing layer disposed upon a surface of the first working electrode;
a membrane disposed over at least the sensing layer; and
a non-electrochemical functional layer comprising a silver salt;
wherein the analyte sensor does not induce cytotoxicity as measured according to ISO 10993-5.

In some embodiments, the silver salt in the non-electrochemical functional layer is silver iodide with a particle size of ≤60 μm and a purity of at least 99%.

In some embodiments, the silver salt in the non-electrochemical functional layer is silver iodide with a maximum particle diameter of ≤60 μm and a purity of at least 99%.

In some embodiments, the silver salt in the non-electrochemical functional layer is silver-silver chloride.

### BRIEF DESCRIPTION OF THE DRAWINGS

The following figures are included to illustrate some aspects of the present disclosure and should not be viewed as exclusive embodiments. The subject matter disclosed is capable of considerable modifications, alterations, combinations, and equivalents in form and function, without departing from the scope of this disclosure.

FIG. 1 shows a diagram of an illustrative sensing system that can incorporate an analyte sensor of the present disclosure.

FIGS. 2A-2C show cross-sectional diagrams of analyte sensors including a single sensing layer.

FIGS. 3A-3C show cross-sectional diagrams of analyte sensors including two sensing layers.

FIG. 4 shows a cross-sectional diagram of an analyte sensor including two sensing layers.

FIGS. 5A-5C show perspective views of analyte sensors including two sensing layers upon separate working electrodes.

FIG. 6 is a fluorescent micrograph image showing a biofilm comprised of microorganisms formed on the sensor tail of an analyte sensor having no antimicrobial quality.

FIG. 7 shows glucose analyte sensor measurements having no antimicrobial quality and reduced sensitivity in the later half to two thirds of the sensor life due to biofilm comprised of microorganisms formed on the sensor tail thereof.

FIGS. 8A-8D show plots of the current response for a sensor coated with a silver-containing membrane polymer against a control.

FIGS. 9A-9E show plots of the current response for a sensor coated with a silver-iodide containing membrane polymer against a control.

FIGS. 10A-10D show plots of the current response for sensors coated with different concentrations of copper-containing membrane polymer against a control.

FIG. 11 is a composite fluorescence and visible light image showing a biofilm comprised of microorganisms formed on the sensor tail of an analyte sensor having no antimicrobial quality.

FIG. 12 is a cross-sectional view depicting a portion of an analyte sensor that is compatible with one or more embodiments of the present disclosure.

FIG. 13 shows a plan view of an implantable analyte sensor that is compatible with one or more embodiments of the present disclosure.

FIG. 14 is a cross-sectional view depicting a portion of an analyte sensor having a membrane that is compatible with one or more embodiments of the present disclosure.

### DETAILED DESCRIPTION

The present disclosure is directed to an analyte sensor comprising a sensor tail substrate having a lower portion and configured for insertion into a tissue; a first working electrode disposed on the lower portion of the sensor tail substrate; a sensing layer disposed upon a surface of the first working electrode; a membrane disposed over at least the sensing layer; wherein the membrane comprises 20% to 50% by weight of an antimicrobial silver salt.

The antimicrobial silver salts can dissolve into ions, for example Ag and I′ ions for silver iodide, which can diffuse from a membrane to the surrounding tissue in which the analyte sensor can be inserted to provide protection against microorganism incursion at or near the sensor tail and the sensing elements thereof. Further, the present disclosure provides a multi-tiered technique (e.g., antimicrobial compounds diffusing from different locations on the sensor tail, combinations of antimicrobial compounds diffusing, different concentrations of antimicrobial compounds diffusing, and the like) for combating microorganism incursion. Thus, increasing tissue health, reducing immune cell infiltration (e.g., by reducing or eliminating host user infection immune response which leads to high immune cell density and tissue encapsulation), and the like. Accordingly, in some instances, the analyte sensors of the present disclosure are themselves designed to be self-preserving (and have increased operating longevity). Moreover, they can be designed to permit the use of the analyte sensors in already infected or wounded tissue, if necessary, without compromising the functionality of the analyte sensor. Diffusion of dissolved ions from the antimicrobial silver salts can occur by any mechanism, examples of which are described hereinbelow.

The present disclosure further provides methods of detecting an analyte using the disclosed sensors and methods of manufacturing the disclosed analyte sensors.

### I. Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification will control.

The articles “a,” “an,” and “the” are used herein to refer to one or to more than one (i.e., to at least one) of the grammatical object of the article. By way of example, “an element” means one element or more than one element.

As used herein, the term “about” means±10% of the specified value, unless otherwise indicated.

The term “at least” prior to a number or series of numbers is understood to include the number associated with the term “at least,” and all subsequent numbers or integers that could logically be included, as clear from context. When “at least” is present before a series of numbers or a range, it is understood that “at least” can modify each of the numbers in the series or range. For example, “at least 3” means at least 3, at least 4, at least 5, etc. When “at least” is present before a component in a method step, then that component is included in the step, whereas additional components are optional.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

As used herein, the terms “measure,” “measuring,” and “measured” can encompass the meaning of a respective one or more of the terms “determine,” “determining,” “determined,” “calculate,” “calculating,” and “calculated.”

As used herein, an “analyte” is a substrate that is subject to be measured or detected. The analyte can be from, for example, a biofluid and can be tested in vivo, ex vivo, or in vitro.

As used herein, a “sensor” is a device configured to detect the presence and/or measure the level of an analyte in a sample via electrochemical oxidation and reduction reactions on the sensor. These reactions are transduced to an electrical signal that can be indicative of presence and be correlated to an amount, concentration, or level of an analyte in the sample.

As used herein, a “working electrode” is an electrode at which the analyte (or additional compound(s) whose level depends on the level of the analyte) is electrooxidized or electroreduced with or without the agency of an electron transfer agent.

As used herein, a “counter electrode” refers to an electrode paired with the working electrode, through which passes a current equal in magnitude and opposite in sign to the current passing through the working electrode. In the context of embodiments of the present disclosure, the term “counter electrode” includes both a) counter electrodes and b) counter electrodes that also function as reference electrodes (i.e., counter/reference electrodes), unless otherwise indicated.

As used herein, a “reference electrode” includes both a) reference electrodes and b) reference electrodes that also function as counter electrodes (i.e., counter/reference electrodes), unless otherwise indicated.

As used herein, “electrolysis” is the electrooxidation or electroreduction of a compound either directly at an electrode or via one or more electron transfer agents.

As used herein, an “electron transfer agent” is a compound that carries electrons between the analyte and the working electrode, either directly, or in cooperation with other electron transfer agents. One example of an electron transfer agent is a redox mediator.

As used herein, a “redox mediator” is an electron-transfer agent for carrying electrons between an analyte, an analyte-reduced or analyte-oxidized, enzyme, and an electrode, either directly, or via one or more additional electron-transfer agents. A redox mediator that includes a polymeric backbone can also be referred to as a “redox polymer.”

A “reactive group” is a functional group of a molecule (e.g., a polymer, a crosslinking agent, an enzyme) that is capable of reacting with another compound to couple at least a portion (e.g., another reactive group) of that other compound to the molecule. Reactive groups include carboxy, activated ester, sulfonyl halide, sulfonate ester, isocyanate, isothiocyanate, epoxide, aziridine, halide, aldehyde, ketone, amine, acrylamide, thiol, acyl azide, acyl halide, hydrazine, hydroxylamine, alkyl halide, imidazole, pyridine, phenol, alkyl sulfonate, halotriazine, imido ester, maleimide, hydrazide, hydroxy, and photo-reactive azido aryl groups. Activated esters, as understood in the art, generally include esters of succinimidyl, benzotriazolyl, or aryl substituted by electron-withdrawing groups such as sulfo, nitro, cyano, or halo groups; or carboxylic acids activated by carbodiimides.

As used herein, a “sensing layer” is a component of the sensor including constituents that facilitate the electrolysis of the analyte. The sensing layer can include constituents such as a redox mediator (e.g., an electron transfer agent or a redox polymer), a catalyst (e.g., an analyte-specific enzyme), which catalyzes a reaction of the analyte to produce a response at the working electrode, or both an electron transfer agent and a catalyst. In some embodiments of the present disclosure, a sensor includes a sensing layer that is non-leachably disposed in proximity to or on the working electrode.

As used herein, a “sensing element” is an application or region of an analyte-specific reactant disposed with the sensing layer. As such, a sensing element is capable of interacting with the analyte. A sensing layer can have more than one sensing element making up the analyte detection area disposed on the working electrode. In some embodiments, the sensing element includes an analyte-specific reactant and an electron transfer agent (e.g., electron transfer agent). In some embodiments, the sensing element includes an analyte-specific reactant, a redox mediator, and a crosslinker.

As used herein, “crosslinking agent” or “crosslinker” is a molecule that contains at least two (e.g., 2, 3, or 4) reactive groups (e.g., terminal functional groups) that can link at least two molecules together (intermolecular crosslinking) or at least two portions of the same molecule together (intramolecular crosslinking). A crosslinking agent having more than two reactive groups can be capable of both intermolecular and intramolecular crosslinkings at the same time.

A “membrane solution” is a solution that contains the components for crosslinking and forming the membrane, including, e.g., polymer (e.g., a modified polymer containing heterocyclic nitrogen groups), a crosslinking agent, and a solvent (e.g., a buffer or an alcohol-buffer mixed solvent).

As used herein, a “biofluid” is any bodily fluid or bodily fluid derivative in which the analyte can be measured. Examples of biofluid include, for example, dermal fluid, subcutaneous fluid, interstitial fluid, plasma, blood (e.g., from a vein or blood vessel), lymph, synovial fluid, cerebrospinal fluid, saliva, bronchoalveolar lavage, amniotic fluid, sweat, or tears.

The term “patient” refers to a living animal, and thus encompasses a living mammal and a living human, for example. The term “user” can be used herein as a term that encompasses the term “patient.”

All ranges disclosed herein are inclusive of the endpoints, and the endpoints are independently combinable with each other. Each range disclosed herein constitutes a disclosure of any point or sub-range lying within the disclosed range. For example, the range from X to Y, is inclusive of X and Y. And, the range between X and Y, is inclusive of X and Y.

The phrase “enzyme composition” refers to a composition that includes one or more enzymes for detecting and/or measuring an analyte. In some non-limiting embodiments, the enzyme compositions can include one or more enzymes, polymers, redox mediators, and/or crosslinkers.

As used herein, the term “antimicrobial” or the phrase “antimicrobial compound,” refer to a substance or material that is detrimental (microbicidal) or microstatic (i.e., preventing or reducing colonization, expansion, and/or proliferation without necessarily being detrimental) to a microorganism, including bacteria, fungi, viruses, protozoans, and the like.

As used herein, the phrase “antimicrobial quality” refers to any mechanism, structure, system, or other technique for imparting any antimicrobial characteristic to a tangible material, including one or more components of the analyte sensors described herein and/or human skin to which the one or more sensor components come into contact.

As used herein, the phrases “upper portion of the sensor tail substrate” or “upper portion of the sensor tail,” refer to a portion of the sensor tail that does not extend beyond the dermis and, generally, includes the top 25% by length of the sensor tail beginning from the proximal-most part of the sensor tail extending from the base of an analyte sensor, encompassing any value and subset therebetween. The “upper portion” of the sensor tail is more proximal to the skin surface than the sensing layer(s) of the sensor tail comprising electrode(s) and analyte sensing layer(s).

As used herein, the phrases “lower portion of the sensor tail substrate” or “lower portion of the sensor tail,” refer to a portion of the sensor tail that extends beyond the upper portion and, generally, includes the bottom 75% by length of the sensor tail beginning from the end of the upper portion to the tip (most distal part) of the sensor tail (or alternatively from the tip of the sensor tail to the upper portion). The “lower portion” of the sensor tail includes at least the sensing layer(s) located at least at or near the tip of the sensor tail and is more distal from the base of the analyte sensor compared to the upper portion thereof. That is, the lower portion is defined as at most 75% by length of the sensor tail beginning at the tip thereof (i.e., beginning at the distal-most part of the sensor tail from the base of the analyte sensor).

### II. Analyte Sensors

### Sensors, Compositions, and Methods of the Disclosure

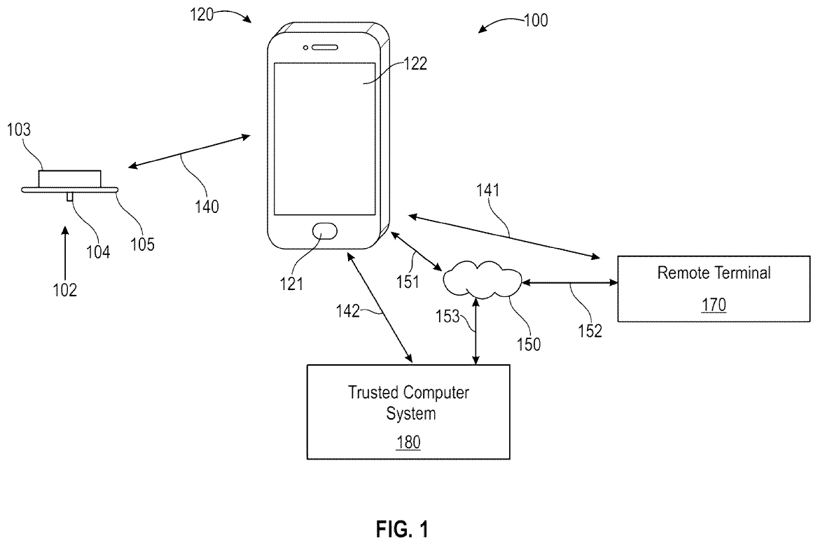

Before describing the analyte sensors of the present disclosure and their components in further detail, a brief overview of suitable in vivo analyte sensor configurations and sensor systems employing the analyte sensors will be provided so that the embodiments of the present disclosure can be better understood. FIG. 1 shows a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to some embodiments. Reader device 120 can be a multi-purpose smartphone or a dedicated electronic reader instrument. While only one reader device 120 is shown, multiple reader devices 120 can be present in some instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to some embodiments, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to some embodiments, by individuals other than a primary user who have an interest in the user's analyte levels. Reader device 120 can include display 122 and optional input component 121. Display 122 can include a touch-screen interface, according to some embodiments.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some embodiments.

Sensor 104 is adapted to be at least partially inserted into a tissue of interest, such as within the dermal or subcutaneous layer of the skin. Sensor 104 can include a sensor tail of sufficient length for insertion to a desired depth in a given tissue. The sensor tail can include at least one working electrode. In some configurations, the sensor tail can include a sensing layer for detecting an analyte. A counter electrode can be present in combination with the at least one working electrode. Particular electrode configurations upon the sensor tail are described in more detail below.

The sensing layer can be configured for detecting a particular analyte. For example, but not by way of limitation, the disclosed analyte sensors include at least one sensing layer configured to detect an analyte (e.g., glucose, ketone). In some embodiments, a sensor of the present disclosure includes two sensing layers, where each sensing layer is configured to detect a different analyte. Alternatively, the two sensing layers can be configured to detect the same analyte. In some embodiments, a first sensing layer can be configured to detect an analyte (e.g., glucose) and a second sensing layer can be configured to detect the first (i.e., same) analyte or a second analyte different from the first analyte (e.g., ketone, creatinine).

In some embodiments of the present disclosure, one or more analytes can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In some embodiments, analyte sensors of the present disclosure can be adapted for assaying dermal fluid or interstitial fluid to determine a concentration of one or more analytes in vivo. In some embodiments, the biological fluid is interstitial fluid.

Referring still to FIG. 1, sensor 104 can automatically forward data to reader device 120. For example but not by the way of limitation, analyte concentration data (i.e., glucose concentration) can be communicated automatically and periodically, such as at a some frequency as data is obtained or after a some time period has passed, with the data being stored in a memory until transmittal (e.g., every minute, five minutes, or other predetermined time period). In some embodiments, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In some embodiments, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In some illustrative embodiments, the introducer can include a needle or similar sharp. As would be readily recognized by a person skilled in the art, other types of introducers, such as sheaths or blades, can be present in alternative embodiments. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more embodiments. After opening the access pathway, the needle or other introducer can be withdrawn so that it does not represent a sharps hazard. In some embodiments, suitable needles can be solid or hollow, beveled or non-beveled, and/or circular or non-circular in cross-section. In some embodiments, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In some embodiments, a tip of the needle (while present) can be angled over the terminus of sensor 104, such that the needle penetrates a tissue first and opens an access pathway for sensor 104. In some embodiments, sensor 104 can reside within a lumen or groove of the needle, with the needle similarly opening an access pathway for sensor 104. In either case, the needle can be subsequently withdrawn after facilitating sensor insertion.

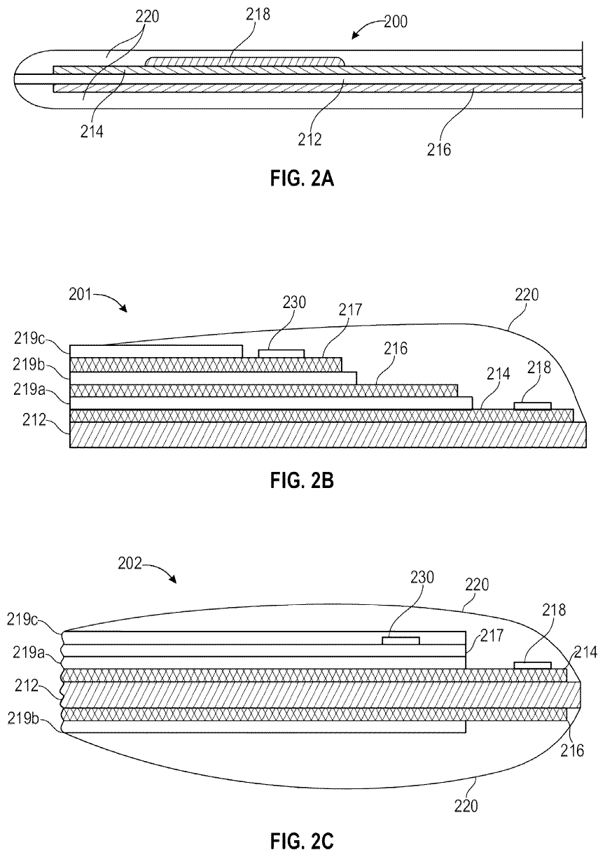

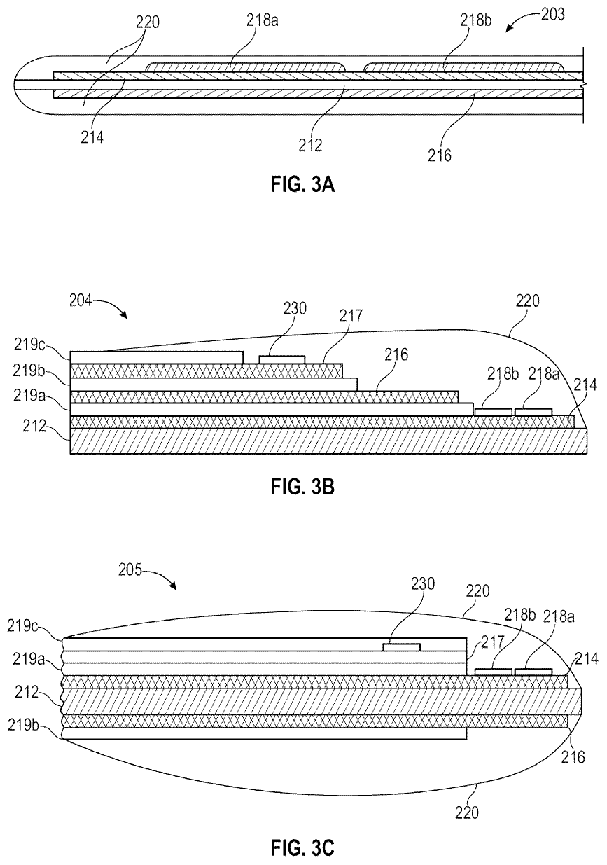

Sensor configurations featuring a single sensing layer that can be configured for the detection of a corresponding single analyte can employ two-electrode or three-electrode detection motifs, as described further herein in reference to FIGS. 2A-2C. Sensor configurations featuring two different sensing layers for detection of separate analytes, either upon separate working electrodes or upon the same working electrode, are described separately thereafter in reference to FIGS. 3A-5C. Sensor configurations having multiple working electrodes can be particularly advantageous for incorporating two different sensing layers within the same sensor tail, since the signal contribution from each sensing layer can be determined more readily.

When a single working electrode is present in an analyte sensor, three-electrode sensor configurations can include a working electrode, a counter electrode, and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the sensor tail. Suitable sensor configurations can be substantially flat in shape, substantially cylindrical in shape or any other suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. 2A shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Sensing layer 218 is disposed as at least one layer upon at least a portion of working electrode 214. Sensing layer 218 can include multiple spots or a single spot configured for detection of an analyte, as discussed further herein.

Referring still to FIG. 2A, membrane 220 overcoats at least sensing layer 218. In some embodiments, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to sensing layer 218 (i.e., membrane 220 is a mass transport limiting membrane having some permeability for the analyte of interest). In some embodiments, and further described below, membrane 220 is not crosslinked. Analyte sensor 200 can be operable for assaying an analyte by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations can be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 2B, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 2C. Thus, in certain embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 2B and 2C. As with sensor 200 shown in FIG. 2A, sensing layer 218 in analyte sensors 201 and 202 can include multiple spots or a single spot. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat sensing layer 218, as well as other sensor components, in analyte sensors 201 and 202, thereby serving as a mass transport limiting membrane. In some embodiments, the additional electrode 217 can be overcoated with membrane 220. Although FIGS. 2B and 2C have depicted electrodes 214, 216, and 217 as being overcoated with membrane 220, it is to be recognized that in some embodiments only working electrode 214 is overcoated. Moreover, the thickness of membrane 220 at each of electrodes 214, 216, and 217 can be the same or different. As in two-electrode analyte sensor configurations (FIG. 2A), one or both faces of analyte sensors 201 and 202 can be overcoated with membrane 220 in the sensor configurations of FIGS. 2B and 2C, or the entirety of analyte sensors 201 and 202 can be overcoated. Accordingly, the three-electrode sensor configurations shown in FIGS. 2B and 2C should be understood as being non-limiting of the embodiments disclosed herein, with alternative electrode and/or layer configurations remaining within the scope of the present disclosure.

FIG. 3A shows an illustrative configuration for sensor 203 having a single working electrode with two different sensing layers disposed thereon. FIG. 3A is similar to FIG. 2A, except for the presence of two sensing layers upon working electrode 214: first sensing layer 218a and second sensing layer 218b, which are responsive to different analytes and are laterally spaced apart from one another upon the surface of working electrode 214. Sensing layers 218a and 218b can include multiple spots or a single spot configured for detection of each analyte. The composition of membrane 220 can vary or be compositionally the same at sensing layers 218a and 218b. First sensing layer 218a and second sensing layer 218b can be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first sensing layer 218a and second sensing layer 218b disposed thereon. FIGS. 3B and 3C are otherwise similar to FIGS. 2B and 2C and can be better understood by reference thereto. As with FIG. 3A, the composition of membrane 220 can vary or be compositionally the same at sensing layers 218a and 218b.

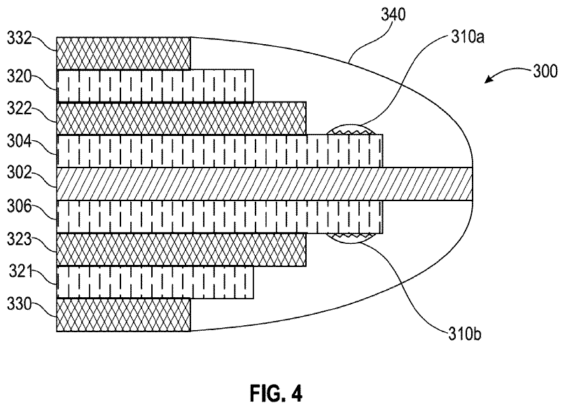

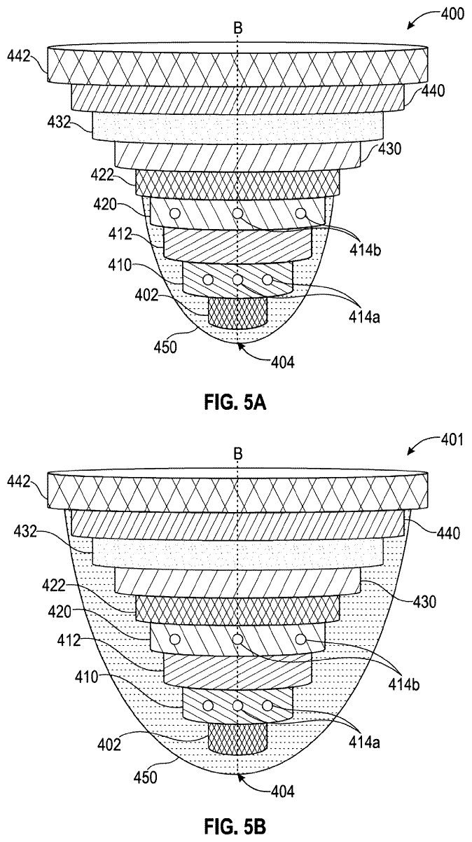

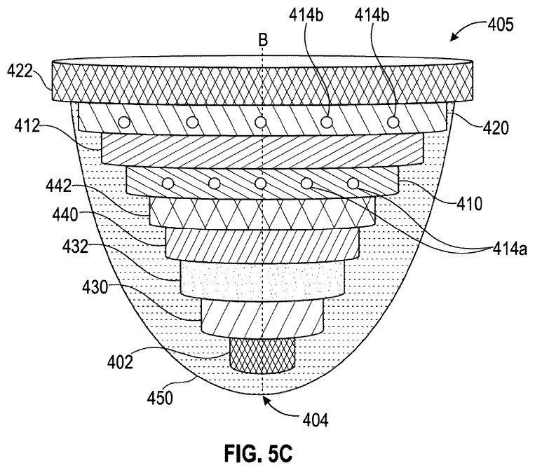

Illustrative sensor configurations having multiple working electrodes, specifically two working electrodes, are described in further detail in reference to FIGS. 4-5C. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte, e.g., for the detection of a third and/or fourth analyte.

FIG. 4 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First sensing layer 310a is disposed upon the surface of working electrode 304, and second sensing layer 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least sensing layers 310a and 310b, according to various embodiments, with other components of analyte sensor 300 or the entirety of analyte sensor 300 optionally being overcoated with membrane 340.

Like analyte sensors 200, 201, and 202, analyte sensor 300 can be operable for assaying an analyte by any of coulometric, amperometric, voltammetric, or potentiometric electrochemical detection techniques.

Alternative sensor configurations having multiple working electrodes and differing from the configuration shown in FIG. 4 can feature a counter/reference electrode instead of separate counter and reference electrodes 320, 321, and/or feature layer and/or membrane arrangements varying from those expressly depicted. For example, and not by the way of limitation the positioning of counter electrode 320 and reference electrode 321 can be reversed from that depicted in FIG. 4. In addition, working electrodes 304 and 306 need not necessarily reside upon opposing faces of substrate 302 in the manner shown in FIG. 4.

Although suitable sensor configurations can feature electrodes that are substantially planar in character, it can be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described herein below. FIGS. 5A-5C show perspective views of analyte sensors featuring two working electrodes that are disposed concentrically with respect to one another. It can be appreciated that sensor configurations having a concentric electrode disposition but lacking a second working electrode are also possible in the present disclosure.

FIG. 5A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 5A, first sensing layers 414a and second sensing layers 414b, which are responsive to different analytes or the same analyte, are disposed upon the exposed surfaces of working electrodes 410 and 420, respectively, thereby allowing contact with a fluid to take place for sensing. Although sensing layers 414a and 414b have been depicted as three discrete spots in FIG. 5A, it can be appreciated that fewer or greater than three spots, including a continuous layer of sensing layer, can be present in alternative sensor configurations.

In FIG. 5A, sensor 400 is partially coated with membrane 450 upon working electrodes 410 and 420 and sensing layers 414a and 414b disposed thereon. FIG. 5B shows an alternative sensor configuration in which the substantial entirety of sensor 401 is overcoated with membrane 450. Membrane 450 can be the same or vary compositionally at sensing layers 414a and 414b.

It can be further appreciated that the positioning of the various electrodes in FIGS. 5A and 5B can differ from that expressly depicted. For example, the positions of counter electrode 430 and reference electrode 440 can be reversed from the depicted configurations in FIGS. 5A and 5B. Similarly, the positions of working electrodes 410 and 420 are not limited to those that are expressly depicted in FIGS. 5A and 5B. FIG. 5C shows an alternative sensor configuration to that shown in FIG. 5B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of sensing layers 414a and 414b (five discrete sensing spots illustratively shown in FIG. 5C), thereby facilitating an increased signal strength in some cases. Similarly, central substrate 402 can be omitted in any concentric sensor configuration disclosed herein, wherein the innermost electrode can instead support subsequently deposited layers.

Several parts of the sensor are further described below.

### III. General Structure of the Analyte Sensor System

In some embodiments, the present disclosure is directed to an analyte sensor comprising:
a sensor tail substrate having a lower portion and configured for insertion into a tissue;
a first working electrode disposed on the lower portion of the sensor tail substrate;
a first sensing layer disposed upon a surface of the first working electrode; and
a membrane disposed over at least the first sensing layer;
wherein the membrane comprises 20% to 50% by weight of an antimicrobial silver salt.

In some embodiments, the present disclosure is directed to an analyte sensor comprising:
a sensor tail substrate having a lower portion and configured for insertion into a tissue;
a first working electrode disposed on the lower portion of the sensor tail substrate;
a first sensing layer disposed upon a surface of the first working electrode, wherein the first sensing layer comprises a first analyte;
a membrane disposed over at least the first sensing layer;
a second working electrode disposed on the lower portion of the sensor tail substrate; and
a second sensing layer disposed upon a surface of the second working electrode and responsive to a second analyte differing from the first analyte;
wherein the membrane comprises 20% to 50% by weight of an antimicrobial silver salt.

### 1. Sensor Tail Substrate

The wear duration of an analyte sensor over an extended period of time (e.g., greater than two weeks, or even longer) can be limited in some instances. For example, analyte sensor chemistry can support long wear times, but there can also be a desire to minimize the risk of infection or biofilm formation at or near the insertion site and the active area of the analyte sensor. Microorganism incursion into or near the operational components of an analyte sensor, such as the membrane or any other active area thereof (i.e., the sensing layer(s)), can result in decreased accuracy and/or other loss of functionality, particularly during extended wear over multiple days or even longer over multiple weeks if microorganism incursion has occurred. In vivo analyte sensors comprise a sensor tail component that can be implanted into a tissue of a user (e.g., transdermally, dermally, subcutaneously, or intravenously) and in some instances, as previously disclosed herein, the sensor tail includes one or more sensing layers at at least the distal tip thereof. As used herein, the term “sensor tail,” refers to the portion of the analyte sensor extending from the base of an external component thereof and of which at least a lower portion can be inserted into the tissue of a user; the sensor tail of the present disclosure typically comprises one or more sensing layers at at least a lower portion thereof (e.g., at or near the distal tip), as described in greater detail herein below. In some embodiments, the sensing layers can be located at a different (e.g., less distal) portion along the sensor tail. Regardless of the implantation qualities of the sensor as a whole (e.g., whether it can be wholly or partially implanted into a tissue of a user), at least a portion of the sensor tail and sensing layer thereof can be in contact with bodily fluid once introduced to a tissue of a user.

Performance of analyte sensors can be highly dependent upon biologic events local to or near the sensor tail. Typically, for accurate analyte measurement, an analyte sensor permits an undisturbed (i.e., consistent or predictable) pathway to communicate with the sensing layer(s); likewise, the sensing layer(s) (and potentially other elements, such as reference materials) must maintain communication with the intended body fluid of interest in an undisturbed or predictable manner. Moreover, for electrochemical sensors, stable connectivity (e.g., electrode connectivity and other electronics) can be necessary to ensure proper analyte sensor functionality. Accordingly, maintaining such pathways and connectivity during the life of an on-body analyte sensor can be critical, including preventing microorganism incursion thereto.

In some embodiments, the present disclosure provides for an analyte sensor comprising a sensor tail substrate. The sensor tail substrate includes a lower portion and an upper portion and can be configured for insertion into a tissue. The tissue can be, for example, a dermal layer, an interstitial layer, or a subcutaneous layer below the surface of the skin. In some embodiments, the tissue can be a subcutaneous layer. The analyte sensor can further comprise at least one electrode disposed on the lower portion of the sensor tail substrate, a sensing layer(s) disposed upon a surface of the electrode, a membrane disposed over at least the sensing layer(s) (and thus, over at least part of the lower portion of the sensor tail substrate), and optionally a non-electrochemical functional layer located at least at the lower portion of the sensor tail substrate; that is, the non-electrochemical functional layer can be provided in the lower portion of the sensor tail substrate at any location and disposed on any other layer, where applicable (e.g., disposed on the lower portion of the sensor tail substrate).

In some embodiments, at least one or more of the lower portion of the sensor tail substrate, the at least one electrode, the sensing layer(s), the membrane, and the optional non-electrochemical functional layer comprises an antimicrobial quality. These can be referred to herein collectively as “sensor tail components.”

In some embodiments, the membrane disposed over at least the sensing layer comprises an antimicrobial quality.

The sensor tail substrate can have an antimicrobial quality, such as by any methods described herein for imparting said antimicrobial quality. In some embodiments, the sensor tail substrate can be doped with or otherwise coated, for example, with one or more antimicrobial compounds, which can leach to the surrounding tissue and/or to other sensor tail components.

In some embodiments, the lower portion of the sensor tail substrate and other components associated therewith, comprising an antimicrobial quality, includes at least all of the lower 75% of the sensor tail substrate extending distally from the tip of the sensor tail (and to the upper portion), encompassing any value and subset therebetween. In some embodiments, the lower portion of the sensor tail and other components associated therewith, comprising an antimicrobial quality, includes at least the lower 50% or at least the lower 25% of the sensor tail substrate extending proximally from the tip of the sensor tail (and toward the upper portion), encompassing any value and subset therebetween. In some embodiments, the lower portion does not comprise the upper portion of the sensor tail (upper 25%), as described herein.

In some embodiments, the sensing layer(s) of an analyte sensor can be within the lower portion of the sensor tail, including less than about 50% of the length of the sensor tail beginning at the tip (or distal-most portion), encompassing any value and subset therebetween. That is, if the sensor tail extending from the base of an external component of an analyte sensor can be determined, the sensing layer(s) can be located at a position that can be within about 50% of the length of the sensor tail beginning from the deepest point within the skin of a user. In some embodiments, the sensing region can be included in less than about 45%, 40%, 35%, 30%, 35%, 20%, 15%, or 10% of the length of the sensor tail beginning at the tip or distal-most portion, encompassing any value and subset therebetween. The location of the sensing region can depend on the length of the sensor tail, the length or area of the sensing layer(s), and the like, and any combination thereof.

In some embodiments, the sensor tail of the analyte sensors described herein can have a length extending from the base of the external component of the analyte sensor within a range of less than about 20 millimeters (mm), such as in the range of about 1 mm to about 15 mm, or about 1 mm to about 7 mm, encompassing any value and subset therebetween. Longer sensor tails are also possible and can be inserted into a tissue at an angle in some embodiments.

The substrate forming a portion of the analyte sensor tails described hereinabove can be flexible or rigid. Suitable materials for a flexible substrate include, but are not limited to, non-conducting plastic or polymeric materials and other non-conducting, flexible, deformable materials. Examples of useful plastic or polymeric materials include thermoplastics such as polycarbonates, polyesters (e.g., MYLAR™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate). Examples of rigid materials that can be used as the substrate include, but are not limited to, poorly conducting ceramics, such as aluminum oxide and silicon dioxide.

The sensor tail can have a plurality of electrodes located at a distal (bottom) end of the sensor tail (e.g., deeper into the subcutaneous space of a user's skin). One or more sensing regions can be associated with (e.g., coated upon or adjacent to) one or more of such electrodes, such as a working electrode. Such electrodes can be co-planar relative to each other, stacked relative to each other, helically wound about one another, incorporated with one or more insulating materials, and the like. The antimicrobial quality can then be provided in any location that imparts antimicrobial protection to the sensor, provided that it is not located at the bottom portion of the sensor tail and it does not interfere with the operation of the sensing region or functionality of the sensor as a whole. That is, in some embodiments, the antimicrobial quality can be exterior to the body or only in contact dermally (i.e., passing through intact skin but not contacting the fatty subcutis layer under the skin) via the upper portion of the sensor tail.

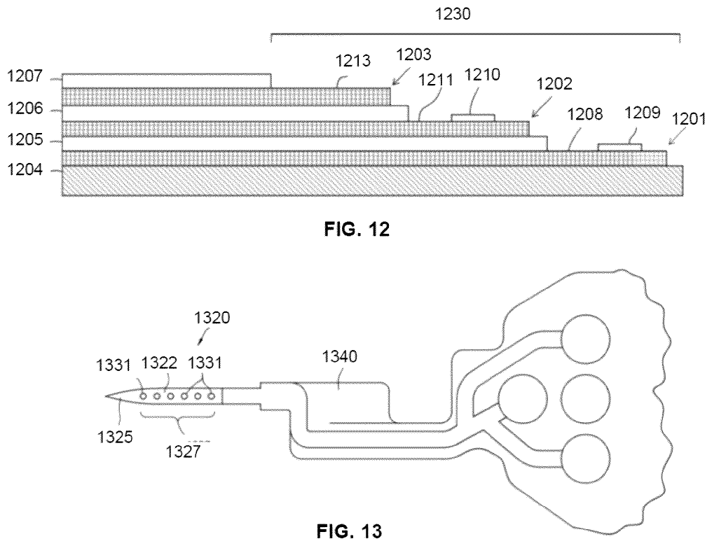

FIG. 12 shows a cross-sectional view of an embodiment of an analyte sensor having a first portion (which in this embodiment can be characterized as a major portion) positionable above a surface of the skin, and a second portion (which in this embodiment can be characterized as a minor portion) that includes a sensor tail 1230 (which can also be referred to herein as an insertion tip) positionable below the surface of the skin (e.g., penetrating through the skin (dermis) and into the subcutaneous space) and in contact with the user's biofluid, such as interstitial fluid. Electrode contacts (not shown) are positioned on the first portion of the sensor 1200 situated above the skin surface and extend to a location in sensor tail 1230. A working electrode 1201, a reference electrode 1202, and a counter electrode 1203 are shown at the bottom portion of sensor tail 1230. It can be understood that greater or fewer electrodes can be provided on a sensor, without departing from the scope of the present disclosure. For example, a sensor can include more than one working electrode and/or the counter and reference electrodes can be a single counter/reference electrode, and the like.

Referring still to FIG. 12, the sensor includes a substrate layer 1204 and a first conducting layer 1208, such as carbon, gold, etc., that can be in electrical communication with sensing area 1209, thereby collectively defining working electrode 1201. Sensing area 1209 can be protected from microorganisms by providing on one or more components of the sensor an antimicrobial quality, designed to protect the skin health of the user and/or to protect the sensing area 1209 from potential interference with such microorganisms (e.g., formation of a biofilm due to potential migration of the microorganisms). The various electrodes and sensing areas defined on the bottom portion of the sensor tail 1230 in FIG. 12 can be collectively a sensing region, and any such antimicrobial quality provided to the sensor tail described herein, can be provided in the upper portion (upper 25%) of the sensor tail 1230 above said region (e.g., above sensing area 1209, or above electrode 1203).

A first insulation layer 1205, such as a first dielectric layer in some embodiments, can be disposed or layered on at least a portion of the first conducting layer 1208, and further, a second conducting layer 1211 can be disposed or stacked on top of at least a portion of the first insulation layer (or dielectric layer) 1205. As shown in FIG. 12, the second conducting layer 1211 in conjunction with a second conducting material 1210, such as a layer of silver/silver chloride (Ag/AgCl), can provide the reference electrode 1202. Another possible disposition of second conducting material 1210 is shown in FIG. 14, along with an outer membrane 1420 overcoating the various layers.

A second insulation layer 1206, such as a second dielectric layer in some embodiments, can be disposed or layered on at least a portion of the second conducting layer 1211. Further, a third conducting layer 1213 can be disposed on at least a portion of the second insulation layer 1206 and can provide the counter electrode 1203. Finally, a third insulation layer 1207 can be disposed or layered on at least a portion of the third conducting layer 1213. In this manner, the sensor can be layered such that at least a portion of each of the conducting layers can be separated by a respective insulation layer (e.g., a dielectric layer). Another possible layer configuration is shown in FIG. 14. The embodiments of FIGS. 12 and 14 show the layers having different lengths; however, some or all of the layers can have the same or different lengths and/or widths, without departing from the scope of the present disclosure.

In any one or all embodiments, some or all of the electrodes 1201, 1202, and 1203 can be provided on the same side of the substrate 1204 in the layered construction described above, or alternatively, can be provided in a co-planar manner such that two or more electrodes can be positioned on the same plane (e.g., side-by side, parallel, or angled relative to each other) on the substrate 1204. For example, co-planar electrodes can include a suitable spacing therebetween and/or include a dielectric material or insulation material disposed between the conducting layers/electrodes. Furthermore, in some embodiments, one or more of the electrodes 1201, 1202, and 1203 can be disposed on opposing sides of the substrate 1204. In such embodiments, contact pads can be on the same or different sides of the substrate. For example, an electrode can be on a first side and its respective contact can be on a second side, for example, a trace connecting the electrode and the contact can traverse through the substrate.

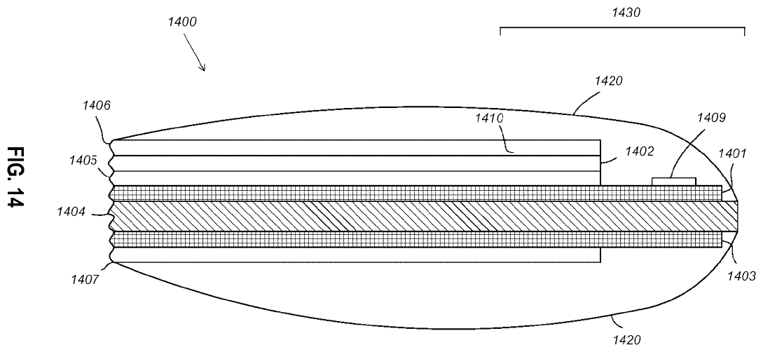

With reference now to FIG. 13, shown is another embodiment of an analyte sensor in accordance with one or more embodiments of the present disclosure, and representing a variation of the sensor of FIG. 12. Referring to FIG. 13, shown is an implantable (e.g., subcutaneous or transcutaneous) sensing region 1320 according to one or more embodiments of the present disclosure including a working electrode 1322. Proximal end 1340 is configured to be connected to various electrical connections for transmitting the output signals of the sensing region 1320. Collectively, the spans between the distal end 1325 and the proximal end 1340 form the sensor tail. Sensing region 1320 encompasses a bottom portion of the sensor tail. As depicted, sensing region 1320 comprises a rounded tip, but other tip shapes can alternately be present to facilitate insertion into a user's skin.

Additionally, in one or more embodiments, sensing region 1320 can include a reference electrode, a counter electrode, or counter-reference electrodes. Alternative electrode configurations can be employed without departing from the scope of the present disclosure.

It is notable that the sensors of FIGS. 12 and 13 include sensing functionality at a distal portion of their respective sensor tails. As described above, this location can allow for enhanced contact with deeper locations beneath a user's skin (e.g., the subcutaneous space), where greater access to the user's interstitial fluid can permit greater access to the analyte of interest being measured (e.g., concentration thereof). That is, the sensing region can be placed sufficiently deep within a user's skin to allow accurate measurement of the particular analyte, whereas placing the sensing region at a more proximate location to the skin surface can be inadequate to correctly determine the concentration or other characteristic of a desired analyte. In some embodiments, the length of the sensor tail can be in the range of less than about 20 millimeters (mm), such as in the range of about 1 millimeters to about 15 mm, or 1 mm to about 6 mm, encompassing any value and subset therebetween. Longer sensor tails can be inserted at an angle in some embodiments.

In some embodiments, the sensing region of an analyte sensor according to one or more embodiments of the present disclosure can be within the distal-most portion of the sensor tail, including less than about 50% of the length of the sensor tail beginning at the distal-most portion, encompassing any value and subset therebetween. That is, if the sensor tail extending from the bottom face 105 (FIG. 1) of a sensor is determined, the sensing region can be located at a position that is within about 50% of the length of the sensor tail beginning from the deepest point within the skin of a user. In other embodiments, the sensing region can be included in less than about 45%, 40%, 35%, 30%, 35%, 20%, 15%, or 10% of the length of the sensor tail beginning at the distal-most portion, encompassing any value and subset therebetween. The location of the sensing region can depend on the length of the sensor tail, the length or area of the sensing region, and the like, and any combination thereof.

The antimicrobial quality of the sensor that can be applied according to one or more embodiments described herein to the upper portion of the sensor tail, located at a distant sufficiently far from the sensing region of the sensor tail such that the antimicrobial does not interfere with the functioning of the sensing region or sensor as a whole, and does not adversely react with the skin of the user (e.g., it is not so deep within the skin to cause a reaction). In some embodiments, if the sensor tail extending from the bottom face 105 (FIG. 1) of a sensor is determined, the antimicrobial quality can be applied by any means or mechanism to the upper (proximal) portion of the sensor tail, including less than about 25% of the length of the sensor tail beginning at the proximal-most portion, encompassing any value and subset therebetween. Some such portion can be located at the insertion site and/or slightly above or below the insertion site, depending on the length of the sensor tail. Accordingly, if the sensor tail extending from the bottom face 105 (FIG. 1) of a sensor is determined, the antimicrobial quality can be located at a position that can be within about 25% of the length of the sensor tail extending from the bottom face 105. In some embodiments, the antimicrobial quality can be included in less than about 25%, 20%, 15%, or 10% of the length of the sensor tail beginning at the proximal-most portion, encompassing any value and subset therebetween. The location of the antimicrobial quality can depend on the length of the sensor tail, the length or area of the sensing region, and the like, and any combination thereof.

Accordingly, in some embodiments, the antimicrobial quality can be at a distance of at least 25% of the length of the sensor tail measured from the bottom face 105 of the sensor away from the sensing region (the “upper portion” of the sensor tail), encompassing any value and subset therebetween increasing in greater distance. That is, the antimicrobial quality and the sensing region can be sufficiently separated such that the two do not interact directly, but instead any pathway to the sensing region along the sensor tail by a microorganism first encounters the antimicrobial quality, which prevents or reduces contact by the microorganism to the sensing region.

In some embodiments, and with reference again to FIG. 1, any of the sensor housing 103, the bottom face 105, the needle, and/or the sensor 104 that protrudes from sensor housing 103 and through bottom face 105 into the skin of a user (i.e., the upper portion of the sensor tail and excluding the sensing region at the insertion tip) can be provided with an antimicrobial quality, including any sub-components thereof (e.g., see FIG. 2A). The antimicrobial quality can be coated thereon, functionalized thereto, impregnated therein (depending on the material or component to which the antimicrobial quality can be imparted), or otherwise adhered thereto to impart the antimicrobial quality to the sensor device 102 and, in particular, for protection of the sensing region of the sensor tail and a user's skin. With reference to FIGS. 1 and 2B, for example, the perimeter of the sensor housing 103 and/or the outer perimeter 202 of the bottom face 105 can be provided with an antimicrobial quality.

### 2. Working Electrode

The at least one electrode which can have an antimicrobial quality can comprise any part or all of one or more working electrode(s), counter electrode(s), reference electrode(s), counter/reference electrode(s), and any combination thereof disposed on the lower portion of the sensor tail substrate. In some embodiments, the at least one electrode can be a working electrode upon which the sensing layer(s) can be disposed. Further, one or more electrodes can be disposed on the lower portion of the sensor tail substrate, including duplicates in type, without departing from the scope of the present disclosure.

In some embodiments, the electrodes can themselves have an antimicrobial quality that can be “activated” through use of the analyte sensor itself. For example, the counter or reference electrode (or combination thereof) can comprise a material that can be reduced or oxidized to provide compounds, elements, or ions that are themselves antimicrobial. As one example, the counter or reference electrode (or combination thereof) can comprise silver/silver iodide (Ag/Agl) the Ag/Agl can be reduced to form silver metal (Ag), silver ions (Ag⁺), and iodide ions (I), with the silver ions and iodide ions providing an antimicrobial quality. As another example, the counter or reference electrode (or combination thereof) can comprise silver/silver chloride (Ag/AgCl) the Ag/AgCl can be reduced to form silver metal (Ag), silver ions (Ag⁺), and chloride ions (Cl⁻), with the silver ions and chloride ions providing an antimicrobial quality. Further, silver ions (Ag⁺) can be released from silver metal to provide additional antimicrobial protection. Accordingly, the electrode can be comprised of any biocompatible antimicrobial compound that also functions as a reference or counter electrode.

In the analyte sensor, the working electrode can be any suitable conductive material, such as carbon, gold, palladium, or platinum. In some embodiments, the working electrode can be a carbon working electrode. In some embodiments, the working comprises at least one sensing layer that senses a desired analyte (e.g., glucose) and can be continuously or discontinuously disposed on at least a portion of the working electrode. A discontinuous application means that the sensing layer forms a discrete shape on the working electrode, such as a spot, a line, or a plurality (i.e., an array) of spots and/or lines. The number of spots or lines is not considered to be particularly limited, but can range from about 2 to about 10, from about 3 to about 8, or from about 4 to about 6. In some embodiments, the number of spots or lines can be 2, 3, 4, 5, 6, 7, 8, 9, or 10. In some embodiments, the sensing layer can be continuous on the working electrode. In some embodiments, the sensing layer can be discontinuous on the working electrode.

### 3. Sensing Layer

In some embodiments, the working electrode can comprise at least one sensing layer. In some embodiments, the working electrode can comprise one sensing layer. In some embodiments, the working electrode can comprise two sensing layers. In some embodiments, the working electrode can comprise a first sensing layer and a second sensing layer, wherein the analyte for the first sensing layer can be different from the analyte for the second sensing layer. In this instance, the first sensing layer and second sensing layer can form an array of multiple spots of each sensing layer, in which some spots sense a first analyte (e.g., glucose) and other spots sense a second analyte different from the first analyte (e.g., ketone, creatinine). Each spot can range in size from about 0.01 to 1 mm² in diameter.

The total size of the sensing layer or areas (combined area of all spots, layers, or sensing layers) can be from about 0.05 mm² to about 100 mm². In some embodiments, the total size can be about 100 mm² or less, about 75 mm² or less, about 50 mm² or less, about 40 mm² or less, about 30 mm² or less, about 25 mm² or less, about 15 mm² or less, about 10 mm² or less, about 5 mm² or less, about 1 mm² or less, or about 0.1 mm² or less. In some embodiments, the total size of the sensing layer or areas ranges from about 0.05 to about 0.1 mm², from about 0.05 to about 100 mm², from about 0.1 to about 50 mm², from about 0.5 to about 30 mm², from about 1 to about 20 mm², or from about 1 to about 15 mm².

The sensing layer or areas can typically have a thickness that ranges from about 0.1 μm to about 10 μm. For example, each sensing layer can be 0.1 μm thick or more (e.g., 0.2 μm or more, 0.3 μm or more, 0.5 μm or more, 0.8 μm or more, 1 μm or more, 2 μm or more, 3 μm or more, 5 μm or more, or 8 μm or more) and typically will have a thickness of 10 μm or less (e.g., 8 μm or less, 5 μm or less, 3 μm or less, 2 μm or less, 1 μm or less, 0.8 μm or less, 0.5 μm or less, 0.3 μm or less, or 0.2 μm or less). In some embodiments, each sensing layer has a thickness from about 0.1 μm to about 10 μm, from about 0.2 μm to about 8 μm, from about 0.5 μm to about 5 μm, from about 1 μm to about 4 μm, or from about 1 μm about 2 μm.

The sensing layer can comprise one or more sensing elements of the analyte sensor. In some embodiments, the sensing layer comprises 1, 2, 3, or 4 sensing elements. In some embodiments, the sensing layer comprises 1 sensing element. In some embodiments, the sensing layer comprises 2 sensing elements. The sensing layer(s) of the analyte sensor can be in contact with the working electrode and located at or near the distal tip of the sensor tail. The sensing element can comprise at least one analyte-responsive enzyme (e.g., a glucose-responsive enzyme), and optionally, a stabilizer, a mediator, and/or a crosslinker.

In some embodiments, a conductive material such as, for example, carbon nanotubes, graphene, or metal nanoparticles, can be combined within the sensing layer or layers to promote rapid attainment of a steady state current. Conductive material can be included in a range from about 0.1% to about 50% by weight (pbw) of the sensing layer (e.g., from about 1 pbw to about 50 pbw, from about 1 pbw to about 10 pbw, or from about 0.1 pbw to about 10 pbw).

According to various embodiments of the present disclosure, a polymer can be present in each sensing layer of the analyte sensors or analyte sensor configurations disclosed herein. Suitable polymers for inclusion in the sensing layer(s) can include, but are not limited to, polyvinylpyridines (e.g., poly(4-vinylpyridine)), polyvinylimidazoles (e.g., poly(l-vinylimidazole)), any mixture thereof, or any copolymer thereof. Illustrative copolymers that can be suitable for inclusion in the sensing layer(s) include those containing monomer units such as styrene, acrylamide, methacrylamide, or acrylonitrile, for example. In illustrative embodiments, the polymer within the active area of the analyte sensors disclosed herein can be a poly(4-vinylpyridine), in which a portion of the monomer units are functionalized with an alkylcarboxylate side chain, a portion of the monomer units are appended to the electron transfer agent with an amido spacer group (see Formula 1 below, for example), and a portion of the monomer units are unfunctionalized. Any combination of the aforementioned polymers can also be used, without departing from the scope of the present disclosure.

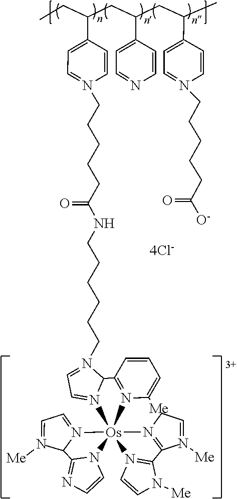

Wherein n is 2, n′ is 17, and n″ is 1.

In some embodiments of the present disclosure, an analyte sensor can include at least one working electrode and at least one sensing layer disposed upon the surface of the working electrode, and at least one redox mediator, e.g., an osmium complex.

### 4. Microorganisms

Microorganisms can disturb the functionality of an analyte sensor in one or more ways. For example, the disturbance can be a chemical disturbance and/or a physical disturbance. Accordingly, the antimicrobial (including microstatic) agents described herein for use in preventing or reducing microorganism interference with the functionality of an analyte sensor can be designed to combat any one or all types of potential microorganism disturbances.

For chemical disturbances, microorganisms can populate the insertion site and influence analyte concentration located adjacent to the sensing layer(s), thereby resulting in false analyte measurement readings. For example, such microorganisms can artificially increase or decrease the analyte level being measured by the sensing layer(s). In certain instances, a microorganism layer (e.g., a dense microorganism layer or biofilm) can consume a portion of the one or more analytes being measured, such as glucose, before it contacts the sensing layer(s) thereof, resulting in an artificially low analyte measurement. In other instances, an analyte sensor can measure a host cell analyte and such analyte can be erroneously measured due to microorganism infection at or near the insertion site, in which the microorganism generates the same analyte as metabolite or other secreted substance (e.g., cytokines, enzymes, etc.). In such instances, the measurement can be artificially high due to an additive effect between the host metabolite level and that of the microorganism. In yet another scenario, microorganisms can interfere with analyte sensor measurement by causing an immune response at or near the insertion site. For example, the measured analyte can be a host cell metabolite and an accumulation of such host cells in response to the infection and can result in an artificially high analyte measurement. Because microorganisms and eukaryotic cells produce acidic metabolites, in another scenario, an analyte sensor that can be pH-sensitive can produce artificially low or high analyte measurements due to microorganism infection or resultant user immune response. That is, the presence of the microorganism or immune response to such can result in increased cell densities and heightened metabolic activity by producing acid, which can reduce pH and lead to false analyte measurements.

For physical disturbances, microorganism incursion, such as infection at the insertion site or along the sensor tail, can result in formation of a biofilm. Biofilms are typically dense networks of microorganism cells (e.g., bacteria cells) embedded in DNA, proteins, polysaccharides, or other compounds and can result in erroneous analyte measurements by an analyte sensor. For example, the biofilm can interfere with diffusion of one or more analytes of interest to the sensing layer(s) of the analyte sensor. In other scenarios, biofouling from protein or other molecule adsorption onto a surface of an analyte sensor, particularly the sensing layer(s) thereof, can also interfere with diffusion of one or more analytes of interest, thereby resulting in artificially low analyte measurements. In certain situations, the membrane of a sensor can become desiccated or otherwise dried due to wound healing (e.g., healing at the insertion site of an analyte sensor), effectively walling off the sensing layer(s) and influencing the functionality of the analyte sensor. Moreover, such membrane desiccation can block the pathway to the sensing layer(s) of the analyte sensor. In some instances, the various electrodes (e.g., working, reference, and/or counter electrodes) can lose connectivity due directly to the microorganism incursion (e.g., due to the desiccation of the membrane or biofilm formation) or indirectly from native immune cell recruitment in response to microorganism incursion.

As previously described, microorganism incursion into an analyte sensor insertion site, including migration at or near the sensing layer(s) disposed on a lower portion of a sensor tail, can result in harm to the user and/or can lead to altered functioning of the analyte sensor itself, including false positive or false negative analyte measurements, shortening the life of the sensor or sensing layer(s), and the like.

Various microorganisms (e.g., bacteria, fungi, viruses, and the like) are present naturally on the skin (i.e., skin flora) and in the surrounding environment (e.g., air, clothing, bedding, water, social engagement, and the like), which can contact the insertion site (e.g., wound area) or adjacent skin while the analyte sensor can be in use and migrate to the sensing layer(s) thereof. Microorganisms on the sensor tail or on skin can be introduced during insertion as well. Moisture contact (e.g., during daily showering, bathing, or submersion (e.g., such as when swimming, for example in a public pool or natural body of water), inability to access and wash skin having the sensor applied thereto, failure to adequately clean the skin prior to insertion of the sensor tail and application of the sensor, nosocomial (i.e., hospital) exposure to microorganisms (e.g., a nurse or other medical professional can apply an analyte sensor in a hospital setting), and the like, can further exacerbate the possibility of microorganism growth. As used herein, the term “skin flora,” and grammatical variants thereof, refers to microorganisms that innately reside on human skin and/or within a human body that can contact human skin. While various embodiments of the present disclosure can be discussed with reference to protecting against such skin flora, it is to be understood that other microorganisms (e.g., bacteria, fungi, viruses, and the like) can also be controlled or otherwise treated to protect at least the sensing layer(s) of an analyte sensor, such as for better sensor functionality and protection of the user.

The various microorganisms that can be desirably controlled or otherwise treated as part of the use of an analyte sensor described herein are not believed to be particularly limited and include any microorganisms generally encountered by a person during various life activities, including symbiotic microorganisms and parasitic microorganisms.

Microorganisms in skin flora can include, but are not limited to, actinobacteria (e.g., corynebacterium, propionibacterium, and the like), firmicutes (e.g., staphylococcus, Clostridia, lactobacillus, and the like), proteobacteria (e.g., alphaproteobacteria, betaproteobacteria, gammaproteobacteria, and the like), bacteriodetes (e.g., flavobacteriales, and the like), cyanobacteria, and the like, and any combination thereof. Examples of specific such skin flora can include, but are not limited to, Staphylococcus epidermidis, Staphylococcus aureus, Staphylococcus warneri, Streptococcus pyogenes, Streptococcus mitis, Staphylococcus hominis, Propionibacterium acnes, corynebacterium spp., Acinetobacter johnsonii, Pseudomonas aeruginosa, Demodex folliculorum, Bacillus oleronius, Bacteroides fragilis, Bacteroides melaninogenicus, Bacteroides oralis, Enterococcus faecalis, Escherichia coli, enterobacter spp., klebsiella spp., Bifidobacterium bifidum, lactobacillus spp., Clostridium perfringens, Clostridium tetani, Clostridium septicum, Proteus mirabilis, Pseudomonas aeruginosa, Salmonella enterica, Faecalibacterium prausnitzii, peptostreptococcus spp., peptococcus spp., and the like, and any combination thereof.

Environmental microorganisms can be any microorganisms that overlap with the skin flora described above, and further include microorganisms that are present in the environment, including internal and external environments and encountered during various life activities. For example, such microorganisms can include those that are encountered during sleeping, bathing and/or showering, working, exercising, eating, attending recreational excursions or events, handling pets, and the like. Such microorganisms can include, but are not limited to, those listed above, as well as escherichia (e.g., E. coli, and the like), firmicutes, mycobacterium (e.g., Mycobacterium avium subspecies paratuberculosis, and the like), enterobacteriaceae (e.g., salmonella, and the like), yeast, bacteriophages, and the like, and any combination thereof.

### 5. Antimicrobial Silver Salt

The embodiments of the present disclosure accordingly impart an antimicrobial silver salt on one or more components of the lower portion of the sensor tail substrate of an analyte sensor in order to reduce or prevent malfunction due to microorganism incursion by incorporating antimicrobial compounds therewith.

In some embodiments, the antimicrobial silver salt can be silver chloride, silver-silver chloride, silver iodide, silver carbonate, silver nitrate, or combinations thereof. In some embodiments, the antimicrobial silver salt can be silver iodide.

In some embodiments, the antimicrobial silver salt can be subjected to sieving to obtain particles with a particle size of ≤60 μm. In some embodiments, the antimicrobial silver salt can be subjected to sieving to obtain particles with a particle size ranging from about 10 μm to about 60 μm, from about 10 μm to about 55 μm, from about 10 μm to about 50 μm, from about 10 μm to about 40 μm, from about 10 μm to about 30 μm, from about 10 μm to about 20 μm, from about 20 μm to about 60 μm, from about 20 μm to about 55 μm, from about 20 μm to about 50 μm, from about 20 μm to about 40 μm, from about 20 μm to about 30 μm, from about 30 μm to about 60 μm, from about 30 μm to about 55 μm, from about 30 μm to about 50 μm, from about 30 μm to about 40 μm, from about 40 μm to about 60 μm, from about 40 μm to about 55 μm, from about 40 μm to about 50 μm, from about 50 μm to about 60 μm, from about 50 μm to about 55 μm, or from about 55 μm to about 60 μm. In some embodiments, the antimicrobial silver salt can be subjected to sieving to obtain particles with a particle size of about 60 μm, about 55 μm, about 50 μm, about 40 μm, about 30 μm, about 20 μm, or about 10 μm. In some embodiments, the antimicrobial silver salt can be subjected to sieving to obtain particles with a particle size of about 53 μm.

In some embodiments, a maximum particle diameter of the antimicrobial silver salt can be obtaining by sieving. The sieve has a square shaped opening of a certain size. The sieve separates larger from smaller particles, distributing the antimicrobial silver salt particles in 2 quantities. The particles with diameters larger than the size of the openings are retained by the sieve, while smaller diameter particles pass through the sieve. A pan collects the smaller diameter particles that pass through the sieve. In some embodiments, the sieve has an opening diameter of less than or equal to 60 μm. In some embodiments, the sieve has an opening diameter of less than or equal to 55 μm. In some embodiments, the sieve has an opening diameter of less than or equal to 53 μm.

In some embodiments, the antimicrobial silver salt can be subjected to sieving to obtain particles with a maximum particle diameter of ≤60 μm. In some embodiments, the antimicrobial silver salt can be subjected to sieving to obtain particles with a maximum particle diameter ranging from about 10 μm to about 60 μm, from about 10 μm to about 55 μm, from about 10 μm to about 50 μm, from about 10 μm to about 40 μm, from about 10 μm to about 30 μm, from about 10 μm to about 20 μm, from about 20 μm to about 60 μm, from about 20 μm to about 55 μm, from about 20 μm to about 50 μm, from about 20 μm to about 40 μm, from about 20 μm to about 30 μm, from about 30 μm to about 60 μm, from about 30 μm to about 55 μm, from about 30 μm to about 50 μm, from about 30 μm to about 40 μm, from about 40 μm to about 60 μm, from about 40 μm to about 55 μm, from about 40 μm to about 50 μm, from about 50 μm to about 60 μm, from about 50 μm to about 55 μm, or from about 55 μm to about 60 μm. In some embodiments, the antimicrobial silver salt can be subjected to sieving to obtain particles with a maximum particle diameter of about 60 μm, about 55 μm, about 50 μm, about 40 μm, about 30 μm, about 20 μm, or about 10 μm. In some embodiments, the antimicrobial silver salt can be subjected to sieving to obtain particles with a maximum particle diameter of about 53 μm.

In some embodiments, the silver iodide salt can be subjected to sieving to obtain particles with a maximum particle diameter of ≤60 μm. In some embodiments, the silver iodide salt can be subjected to sieving to obtain particles with a maximum particle diameter ranging from about 10 μm to about 60 μm, from about 10 μm to about 55 μm, from about 10 μm to about 50 μm, from about 10 μm to about 40 μm, from about 10 μm to about 30 μm, from about 10 μm to about 20 μm, from about 20 μm to about 60 μm, from about 20 μm to about 55 μm, from about 20 μm to about 50 μm, from about 20 μm to about 40 μm, from about 20 μm to about 30 μm, from about 30 μm to about 60 μm, from about 30 μm to about 55 μm, from about 30 μm to about 50 μm, from about 30 μm to about 40 μm, from about 40 μm to about 60 μm, from about 40 μm to about 55 μm, from about 40 μm to about 50 μm, from about 50 μm to about 60 μm, from about 50 μm to about 55 μm, or from about 55 μm to about 60 μm. In some embodiments, the silver iodide salt can be subjected to sieving to obtain particles with a maximum particle diameter of about 60 μm, about 55 μm, about 50 μm, about 40 μm, about 30 μm, about 20 μm, or about 10 μm. In some embodiments, the silver iodide salt can be subjected to sieving to obtain particles with a maximum particle diameter of about 53 μm.

In some embodiments, the antimicrobial salt has a purity ranging from about 80% to about 99%. In some embodiments, the antimicrobial salt has a purity ranging from about 80% to about 99%, from about 80% to about 98%, from about 80% to about 97%, from about 80% to about 96%, from about 80% to about 95%, from about 80% to about 90%, from about 80% to about 85%, from about 85% to about 99%, from about 85% to about 98%, from about 85% to about 97%, from about 85% to about 96%, from about 85% to about 95%, from about 85% to about 90%, from about 90% to about 99%, from about 90% to about 98%, from about 90% to about 97%, from about 90% to about 96%, from about 90% to about 95%, from about 95% to about 99%, from about 95% to about 98%, from about 95% to about 97%, from about 95% to about 96%, from about 96% to about 99%, from about 96% to about 98%, from about 96% to about 97%, from about 97% to about 99%, from about 97% to about 98%, or from about 98% to about 99%. In some embodiments, the antimicrobial salt has a purity of about 99%.

Some embodiments of the present disclosure utilize a membrane as a delivery system or substrate (e.g., carrier) for delivering an antimicrobial silver salt and, therefore, reducing or preventing erroneous analyte measurements of an analyte sensor due to one or more of the microorganism disturbances described above.

The antimicrobial silver salt can be disposed over one or more components of the lower portion of the sensor tail substrate by any means suitable including, but not limited to, intermixing with an antimicrobial silver salt (i.e., mixing any one or more antimicrobial silver salts with the sensor tail components without chemical binding), coating an antimicrobial silver salt onto a surface of one or more of the sensor tail components (e.g., spray coating, dip coating, screen print coating, 3D print coating, thin layer coating, powder coating, press-forming, and the like), or chemical binding with an antimicrobial silver salt (e.g., covalent bonding, ionic bonding, metallic bonding, hydrogen bonding, polar bonding, van der Waals bonding, functionalized, and the like) to the one or more sensor tail components, including the sensor tail substrate, one or more electrodes (e.g., working, reference, and/or counter electrode), the sensing layer(s), the membrane(s), and any optional non-electrochemical functional layer and/or dielectric layer of the analyte sensor. As discussed in greater detail below, the antimicrobial silver salts can themselves further be encapsulated or impregnated, without departing from the scope of the present disclosure.

In some embodiments, for example, the antimicrobial silver salt can be intermixed within a membrane (e.g., homogeneously or spatially intermixed (e.g., heterogeneously, such as in a gradient, or in pockets, or only on a surface layer or dip layer, or on a specific side, or only within a specified depth range, and the like, and any combination thereof)), added to the sensor tail substrate below the membrane (e.g., sensor tail substrate or a printed region (typically carbon), including any of the substrate, sensing layer(s), dielectric layer(s), electrode(s), or any other component of the analyte sensor), coated directly on the membrane surface, printed directly onto the membrane, and/or chemically bound (e.g., covalently bound) to the surface of the substrate or membrane. Spatially intermixed antimicrobial silver salts are accordingly tuned and purposely localized such that a desired release profile, localized release profile, or localized contact with tissue or an element of the analyte sensor can be achieved. In some embodiments, surface coatings (e.g., thin film or thin layer coatings) of the antimicrobial silver salt that impart an antimicrobial quality to the one or more components of the sensor tails described herein can be applied to produce a chemistry layer that can be unfavorable to microorganism colonization and/or proliferation (e.g., biofilm formation, colony formation, increased colony-forming units (CFUs)) and/or microorganism attachment (e.g., particular surface energy, hydrophobicity, hydrophilicity, micro-topography, and/or nano-topography, and the like, and any combination thereof).

The antimicrobial silver salts can diffuse from the one or more components of the sensor tail to the surrounding tissue in which it can be inserted to provide protection against microorganism incursion at or near the sensor tail and sensing layer(s) thereof. Further, the present disclosure provides a multi-tiered technique (e.g., antimicrobial compounds diffusing from different sensor tail locations, combinations of antimicrobial compounds diffusing, different concentrations of antimicrobial silver salts diffusing, and the like) for combating microorganism incursion. Thus, increasing tissue health, reducing immune cell infiltration (e.g., by reducing or eliminating host user infection immune response which leads to high immune cell density and tissue encapsulation), and the like. Accordingly, in some embodiments, the analyte sensors of the present disclosure are themselves designed to be self-preserving (and have increased operative longevity). Moreover, they can be designed to permit the use of the disclosed analyte sensors in already infected or wounded tissue, if necessary, without compromising the functionality of the analyte sensor. Diffusion of the antimicrobial silver salts can occur by any mechanism, examples of which are described hereinbelow.

The mechanism by which the antimicrobial silver salt described herein prevents or reduces microorganism interference with the insertion site and/or functioning of the analyte sensor can be any mechanism, and can be dependent on the type or types of microorganisms being targeted. Suitable mechanisms can include, but are not limited to, preventing microorganism attachment to one or more components of the sensor (e.g., preventing initial adhesion of the microorganism(s)), disruption of the microorganism(s) cell surface or membrane, disruption of the microorganism(s) internal organelles or functioning thereof, prevention of continued growth (e.g., by restricting nutrients and/or nutrient uptake or metabolism), and the like, and any combination thereof. In some instances, the mechanism can further be selected based on the placement of the antimicrobial silver salt, which can be applied or otherwise coated onto one or more components of the sensor. Optionally, the antimicrobial silver salt can be imparted to one or more components of the analyte sensor and/or particular area of the skin to which the sensor can be adhered in the form of a wipe or cleaning pad, which can be used alone or in combination with other cleaning aspects, such as for imparting disinfecting qualities prior to sensor implantation.

In some embodiments, the antimicrobial silver salt for use in the present disclosure can be designed to slowly release an antimicrobial silver salt. The mechanism by which the antimicrobial silver salt can be slowly released is not considered to be particularly limited. In some embodiments, the antimicrobial silver salt can be released slowly by contact with water (e.g., bodily fluids), exposure to elevated temperature, encapsulation, impregnation, carrier degradation, bond degradation/cleavage, and the like, and any combination thereof. As used herein, the term “encapsulation,” refers to envelopment in whole or in part (i.e., less than 100%) of an antimicrobial silver salt, such as by a biocompatible wax, degradable biopolymer, degradable polymer, or other suitably biocompatible and degradable material. The term “impregnation,” as used herein, refers to filling, permeating, doping, chemically binding (e.g., covalently binding), or saturating of an antimicrobial compound into a carrier, such as an biocompatible alcohol, a biocompatible nanoparticle, and the like. Encapsulated antimicrobial silver salts can generally be released as the encapsulation material degrades or at least are released in greater amounts as such degradation occurs (e.g., if the encapsulation is not in whole). Impregnated antimicrobial silver salts can generally be released as the antimicrobial silver salt leaches out of voids in various carrier materials or as the carrier materials degrade.

Prolonged exposure to an antimicrobial silver salt can be particularly advantageous for the embodiments of the present disclosure because the analyte sensor can be worn over an extended period of time, and thus can experience potential contact with microorganisms, such as skin or environmental flora, over that period of time. Accordingly, the action of the antimicrobial silver salt can occur throughout the duration in which the sensor is in use, not merely at a beginning portion, thereby ensuring protection to the user and proper functioning of the analyte sensor.

In some embodiments, one or more antimicrobial silver salts are homogeneously or spatially intermixed with one or more components of the sensor tail, such as the membrane (or the electrode ink (e.g., reference electrode material or ink), dielectric ink, non-electrochemical functional layer, or sensing layer(s), and the like, and any combination thereof) to achieve prolonged, localized, and/or sequential diffusion of the antimicrobial silver salt(s). For example, homogenous intermixing of an antimicrobial silver salt(s) can cause sustained delivery diffusion such that the antimicrobial silver salt slowly leaches from the sensor tail component over time. In some embodiments, the antimicrobial silver salt(s) can be spatially intermixed such that the tissue surrounding certain portions of the sensor tail or certain portions of the sensing layer(s) receive a greater dosage of the antimicrobial silver salt. For example, the entirety of the lower portion of a sensor tail can have a membrane coating, where only some of the membrane coats the sensing layer(s) and the remaining membrane does not coat the sensing layer(s) (e.g., only coats the sensor substrate or one or more electrodes). In such instances, the antimicrobial silver salt can be designed to be spatially intermixed such that a greater amount of antimicrobial silver salt can be associated with the membrane portion coating the sensing layer(s) and a lesser amount of antimicrobial silver salt can be associated with the membrane portion that is not coating the sensing layer(s).

In some embodiments, the antimicrobial silver salt can be provided such that diffusion into the surrounding tissue can be in the form of a bolus or, alternatively, a sustained (e.g., prolonged) diffusion. The bolus can be a single dose of antimicrobial silver salt(s) that diffuses in total or a large diffusion followed by residual diffusion, either of which can be achieved, for example, by the spatial intermixing discussed above. In some embodiments, the proximal layers of the one or more sensor tail components relative to the tissue of a user (i.e., farther from the sensor tail substrate) can include the antimicrobial silver salt in greater amount compared to the more distal layers to the tissue, such that greater concentration of the antimicrobial silver salt can be diffused into the surrounding tissue during the initial or earlier duration of the life of the analyte sensor compared to later. Alternatively, the proximal layers of the one or more sensor tail components relative to the tissue of a user can include the antimicrobial silver salt in lesser amount compared to the more distal layers to the tissue, such that as the antimicrobial silver salt is exhausted in the proximal layers, it can be replenished by the more distal layers.

Alternatively, localized placement of one or more antimicrobial silver salts can be provided such that delivery of a particular antimicrobial silver salt occurs prior to delivery of a second (or third, etc.) antimicrobial compound. For example, it can be known that a particular microorganism is likely to be most problematic during the initial implantation of an analyte sensor, followed by a different microorganism over time; the controlled release of two or more antimicrobial compounds in accordance with the embodiments described herein can be provided to address such a situation. In some embodiments, the combination of multiple antimicrobial compounds can be used simultaneously to combat against a larger spectrum of microorganisms as compared to any one alone (e.g., the use of rifampin and minocycline together). In some embodiments, the localized placement of various antimicrobial compound(s) can be used to create a wave-like effect where higher doses of antimicrobial compound are followed by lower or no antimicrobial compound dosage. In some embodiments, the amount and type of antimicrobial compound diffusing to the tissue adjacent to the implanted sensor tail can be controlled to achieve desired tolerance levels (e.g., lower drug load to the user). Accordingly, the present disclosure provides for multiple release profiles of the antimicrobial silver salts described herein.

In some embodiments, the sensor tail components themselves act as the media through which the antimicrobial silver salt diffuses to the surface of the particular component, potentially through other components when appropriate (e.g., depending on the relationship between the components), and to the surrounding tissue at the insertion site of a user. For example, in some embodiments and as described above, the membrane of the analyte sensors themselves are used as a means of diffusing one or more antimicrobial compounds to both the surface of the membrane (and, typically, also the surface of the sensor tail comprising the sensing layer(s)) and to surrounding tissue. The membrane already functions as a mass transport limiting membrane and, accordingly, the selected antimicrobial compounds can be based on the composition of the membrane to customize diffusion of the antimicrobial compound, thereby maximizing qualities already part of the analyte sensor.

In some embodiments, the antimicrobial compound can be imparted to the sensor tail components described herein, where the sensor tail components act as acceptable substrates for receiving such antimicrobial compound via an antimicrobial coating. An “antimicrobial coating,” as described herein, refers to a film or layer of antimicrobial applied to a surface by any means including, but not limited to, through use of an adhesive, through surface functionalization (e.g., crosslinking, graft polymer linking, and the like of the surface and the antimicrobial), screen printing or other printing mechanisms, spray coating, and the like. Carriers can be used that, when applied to a surface, can themselves be degradable and allow a slow-release (e.g., upon water exposure, elevated temperatures, and the like over time) of an antimicrobial compound, the antimicrobial compound of which can itself be degradable or otherwise encapsulated or impregnated in another material, as previously discussed.

Suitable carriers that can be coated onto one or more of the surfaces of the sensor tail components described herein are not considered to be particularly limited and include any such substances that have or can carry an antimicrobial compound, adhere to a desired surface, and are themselves able to release the antimicrobial compound over time.

In some embodiments, a suitable coating carrier for use in the present disclosure can include, but is not limited to, synthetic and natural biodegradable polymers. Suitable synthetic biodegradable polymer coating carriers can include, but are not limited to, poly(lactic-co-glycolic acid); polylactic acid; polyglycolic acid; polyethylene glycol; poly(D,L) lactide; poly(s-caprolactone); polyhydroxyalkonate; poly(butylene succinate); polyvinyl alcohol; degradable polyurethanes; and the like; any derivatives thereof; any copolymers thereof; and any combination thereof. Suitable natural biodegradable polymer coating carriers for use in the present disclosure can include, but are not limited to, cellulose, chitosan, chitin, lignin, pullulan, polyhydroxyalkonate, collagen, alginate, whey protein, keratin, gelatin, dextran, starch, silk, any derivatives thereof, any salt thereof, any copolymers thereof, and any combination thereof. It can be appreciated that any suitable other degradable synthetic or natural polymers or proteins can be used as an antimicrobial carrier coating in accordance with any or all of the embodiments described herein, without departing from the present disclosure. As used herein, the term “derivative,” refers to a compound that is derived from any one of the listed compounds herein, such as by replacement of one atom with another atom or group of atoms (e.g., a functional group). For instance, examples of cellulose derivatives can include, but are not limited to, hydroxyethylcellulose, carboxymethylhydroxyethylcellulose, carboxymethylcellulose, hydroxypropyl methylcellulose, and the like, and any combination thereof. The term “copolymer,” as used herein, refers to any polymer characterized by two or more different monomers, encompassing terpolymers and any higher polymers.

In some embodiments, the carrier coating itself can provide the antimicrobial quality (e.g., the non-electrochemical functional layer, in some cases, for example). Such antimicrobial carriers can additionally carry a secondary (or tertiary, or any plurality) antimicrobial compound, without departing from the scope of the present disclosure. For example, an organosilane coating (e.g., nanocoating) can provide an abrasive surface to a sensor tail component, which inhibits microorganism attachment due to disruption of their outer membrane. The abrasive surface can be undetectable to humans, and thus suitable for use in the sensor components located in at least the lower portion of the sensor tail inserted into a tissue. In some instances, the organosilane can be coupled with a quaternary ammonium compound, which provides additional anchoring to a surface, such as by forming a chemical bond with one or more components of the sensor tail. Moreover, a quaternary ammonium compound can be used alone if desired to provide an antimicrobial quality to the sensor and/or skin.

A selected organosilane for use as the carrier coating and/or the antimicrobial compound itself is not considered to be particularly limiting and can include any suitable carrier coating that can carry an antimicrobial compound and/or provide antimicrobial qualities itself over time. In some embodiments, examples of suitable organosilane carrier coatings for use in the present disclosure can include, but are not limited to, methyl triethoxysilane, methyl trimethoxysilane, vinyl trimethoxysilane, phenyl trimethoxysilane, 3-aminopropyltrimethoxysilane, 3-glycidoxypropyltrimethoxysilane, methacryloxypropyltrimethoxysilane, mercaptopropyltrimethoxysilane, and the like, any derivative thereof, and any combination thereof.

A selected quaternary ammonium compound for use as the carrier coating and/or the antimicrobial compound itself (e.g., alone or in combination with an organosilane) is not considered to be particularly limited. Examples of suitable quaternary ammonium compounds for use in the present disclosure can include, but are not limited to, dimethyloctadecyl (3-trimethoxysilyl propyl) ammonium chloride, alkyldimethylbenzylammonium chloride, didecyldimethylammonium chloride, benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, quaternary ammonium polyethyleneimine, and the like, any derivatives thereof, and any combination thereof.

The attachment of the quaternary ammonium compound described above can be considered a surface functionalization of one or more components of the sensor described herein for imparting an antimicrobial quality to which it is covalently bound. Other carrier coatings comprising an antimicrobial quality can additionally be achieved by surface functionalization, without departing from the scope of the present disclosure. For example, a surface of one or more components of the sensor tail (e.g., the lower portion of the sensor tail) as described herein can be functionalized by attaching a polymer or polypeptide to the surface, such as through a grafting process. Polymers can be grafted (e.g., through use of absorption or chemical bonding, crosslinking, or other immobilization means) or directly polymerized onto one or more surfaces of the sensor. Such polymers can form a matrix for acting as a carrier coating for one or more antimicrobial compounds in which the antimicrobial can be free-floating (i.e., distributed without chemical bonding throughout the matrix). In other embodiments, the polymers can be functionalized to allow direct bonding with one or more antimicrobial compounds (e.g., to have quaternary amine functional groups).

In some embodiments, the polymer coatings can additionally provide an antimicrobial quality themselves. Suitable such polymers for use in the embodiments of the present disclosure that can provide antimicrobial qualities can include, but are not limited to, those polymers specified above, an organosilane, a quaternary ammonium compounds, 4-vinyl-n-hexylpyridinium bromide, dimethyl(2-methacryloyloxyethyl) phosphonate, poly(ethylene glycol), poly(sulfobetaine methacrylate), poly[3-dimethyl(methacryloyloxyethyl) ammonium propane sulfonate-b-2-(diisopropylamino)ethyl methacrylate], poly(2-methyl-2-oxazoline), a polyphenol, polyhexadine, a chlorohexidine polymer, a nisin-immobilized organosilicon, poly(n,n-diethylethylenediamine-co-aerosol-based acrylic), a benzimidazole polymer, a halogen polymer, a N-halamine polymer, quaternary phosphonium modified epoxidized natural rubber, arginine-tryptophan-rich peptide, guanylated polymethacrylate, polyguanidine, olethyleneimine, chitosan, chitosan derivatives, ammonium ethyl methacrylate homopolymer, metallo-terpyridine carboxymethyl cellulose, poly(N-vinylimidazole) modified silicone rubber, poly-ε-lysine, cationic quaternary polyelectrolytes, 2-(dimethylamino)ethyl methacrylate, a benzaldehyde, 5-chloro-8-hydroxy-quinoline polymers, and the like, any derivatives thereof, any copolymers thereof, and any combination thereof.

One or more antimicrobial compounds can be encapsulated or impregnated in a slow-release compound where the antimicrobial can be at least partially surrounded by the encapsulating material or permeating void space (e.g., pores) of an impregnatable material, respectively. Both of the encapsulating material and the impregnatable material can be degradable to allow slow release of the antimicrobial. In some embodiments, the impregnatable material is not degradable, but instead the antimicrobial can be able to leach from the voids of the impregnatable material to provide the desired antimicrobial quality and amount of antimicrobial dosing, without degradation of the impregnatable material itself. Such compounds should be biocompatible and not negatively interfere with the functioning of the analyte sensor or the health of the surrounding tissue.

Moreover, the encapsulated and/or impregnated antimicrobial compounds can be incorporated into any of the compounds used to form the sensor tail components (e.g., the electrode ink (e.g., reference electrode material or ink), the sensing layer(s), the membrane, the substrate, the dielectric, the non-electrochemical functional layer(s)) or coatings listed above by chemical means (e.g., chemical bonding) or by dispersion (e.g., without chemical bonding or by matrix stabilization), without departing from the scope of the present disclosure. In other embodiments, the encapsulated and/or impregnated antimicrobial compounds can themselves allow covalent or other, such as associative, bonding with one or more components of the sensor tail described herein, depending on the composition of the encapsulating material and/or impregnatable material, the composition of the sensor component, and the like.

An encapsulating material for use in the present disclosure is not considered to be particularly limited provided that as the material degrades and releases the encapsulated antimicrobial compound it does not interfere with the tissue health of a user or the functioning of the analyte sensor described herein, and can generally be any biocompatible, degradable material known to one of skill in the art. In some embodiments, the encapsulating material itself can be able to provide an antimicrobial quality. Examples of suitable encapsulating materials for use in the embodiments of the present disclosure can include, but are not limited to, the degradable synthetic and natural polymers and proteins described herein, biocompatible or biodegradable ceramics, metallic biocompatible or biodegradable compounds, biocompatible or biodegradable micelle-forming materials (i.e., for micelle encapsulation of the antimicrobial, such as biocompatible or biodegradable polymeric micelle materials), and the like, and any combination thereof. Examples of biocompatible or biodegradable ceramics for use in the embodiments described herein can include, but are not limited to, calcium sulfate, calcium carbonate, calcium phosphates, dicalcium phosphates, tricalcium phosphates, hydroxyapatite, and the like, and any combination thereof. Examples of metallic biocompatible or biodegradable compounds for use in the embodiments described herein can include, but are not limited to, magnesium-based alloys such as magnesium alloyed with calcium, zinc, aluminum, manganese, indium, silver, zirconium, and the like, and any combination thereof.

An impregnatable material for use in the present disclosure is not considered to be particularly limited provided that the material can be able to release an antimicrobial and does not interfere with the tissue health of a user (i.e., maintains skin health) or the functioning of the analyte sensor described herein, and can generally be any biocompatible material known to one of skill in the art for placement of an antimicrobial. In some embodiments, the impregnatable materials can be generally porous or have areas of void space for filling or penetration by an antimicrobial compound. In some embodiments, the impregnatable materials can be inclusion complexes, such as cyclodextrins (e.g., alpha, beta, or gamma), which can be particularly compatible with a hydrophobic antimicrobial compound (e.g., certain peptides, hydrophobically functionalized polymers, and the like) and which can be released from the cyclodextrin complex upon contact with water, for example, because the outer surface thereof is hydrophilic.

Suitable impregnatable materials can be any of the biocompatible materials, including those that are degradable and those that themselves impart antimicrobial qualities, described herein, and have an antimicrobial permeated therethrough. Some such materials can be made porous and have an antimicrobial permeated therethrough. For example, an antimicrobial can be impregnated into void space of a cellulose or other natural polymer, a metal, a synthetic polymer, a metal oxide, and the like, and any combination thereof.

In some embodiments, the impregnatable material can be a nanoparticle composed of one or more of the materials described herein impregnated (or, in some embodiments, made wholly from) one or more antimicrobial compounds, which can be made from any of the biocompatible materials described herein and/or any biocompatible materials known to those of skill in the art. As used herein, the term “nanoparticle,” and grammatical variants thereof, refers to a particle having a diameter in the range of about 1 nanometer (nm) to about 1000 nm, encompassing any value and size distribution therebetween. Preferably, a nanoparticle described herein has a diameter in the range of about 1 nm to about 100 nm, encompassing any value and size distribution therebetween. For example, an antimicrobial component can be permeated in void space within or on a nanoparticle (or other impregnatable material) by any means such as chemical deposition (e.g., chemical reduction), sonochemical deposition, solvent impregnation (e.g., supercritical solvent deposition), microwave deposition, simple diffusion (e.g., contained by surface interactions), absorption, adsorption, or any other mechanism known to one of skill in the art. In some embodiments, the nanoparticles can be incorporated with one or more coatings to impart antimicrobial quality. Alternatively, the nanoparticles can themselves have sufficient attraction to one or more surfaces of the sensor tail components (e.g., located at least in the lower portion) to achieve a thin film applied thereupon.

In vivo analyte sensors can include a membrane disposed over at least a portion of the implanted portion of the analyte sensor. In some embodiments, the antimicrobial membrane can improve biocompatibility of the analyte sensor. In some embodiments, the antimicrobial membrane can be permeable or semi-permeable to an analyte of interest and limit the overall analyte flux to the active area of the analyte sensor (i.e., sensing layer(s)), such that the antimicrobial membrane also functions as a mass transport limiting membrane. Limiting analyte access to the sensing layer(s) of the analyte sensor with a mass transport limiting membrane can aid in avoiding sensor overload (saturation), thereby improving detection performance and accuracy. Such membranes can be highly specific toward limiting mass transport of a particular analyte, with other substances permeating through the membrane at significantly different rates, reducing background and interference signals from non-specific redox reactions with analyte molecules other than those of interests. In some embodiments, the antimicrobial membrane can aid in retaining a cofactor (e.g., NAD or NADP) within a sensing layer while still permitting sufficient inward diffusion of the analyte to permit detection thereof.

The mechanism by which the antimicrobial quality described herein prevents or reduces microorganism interference with the insertion site, tissue surrounding the sensor tail, and/or functioning of the analyte sensor can be any mechanism and can be dependent on the type or types of microorganisms being targeted. Examples of such mechanisms can include, but are not limited to, preventing microorganism attachment to one or more components of the sensor tail (e.g., preventing initial adhesion of the microorganism(s)), disruption of the microorganism(s) cell surface or membrane, disruption of the microorganism(s) internal organelles or functioning thereof, prevention of continued growth (e.g., by restricting nutrients and/or nutrient uptake or metabolism), and the like, and any combination thereof. In some embodiments, the mechanism can further be selected based on the placement of the antimicrobial compound, which can be applied or otherwise imparted to one or more components of the sensor tail described herein.

The antimicrobial compounds for use in the present disclosure include all biocompatible antimicrobial compounds, including those described hereinabove, regardless of their particular form (e.g., coating, particle, and the like), and including an organosilane, a quaternary ammonium, the antimicrobial polymers, and the like, and any combination thereof.

The particular amount of antimicrobial provided to any one or more components of the sensor tail can depend on a number of factors including, but not limited to, the physiology and daily routine of a user, the component(s) of the sensor tail to which the antimicrobial silver salt can be imparted, the particular antimicrobial mechanism and compound selected, and the like.

In some embodiments, the antimicrobial silver salt can be present in an amount by weight of a polymer membrane before crosslinking from about 20% to about 50%, from about 20% to about 45%, from about 20% to about 40%, from about 20% to about 35%, from about 20% to about 30%, from about 20% to about 25%, from about 25% to about 50%, from about 25% to about 45%, from about 25% to about 40%, from about 25% to about 35%, from about 25% to about 30%, from about 30% to about 50%, from about 30% to about 45%, from about 30% to about 40%, from about 30% to about 35%, from about 35% to about 50%, from about 35% to about 45%, from about 35% to about 40%, from about 40% to about 50%, from about 40% to about 45%, or from about 45% to about 50%. In some embodiments, the antimicrobial silver salt can be present in amount by weight of a polymer membrane before crosslinking of about 20%, about 25%, about 30%, about 35%, about 40%, about 45%, or about 50%.

In some embodiments, the antimicrobial silver salt can be present in an amount by weight of the polymer membrane 10Q5 before crosslinking from about 20% to about 50%, from about 20% to about 45%, from about 20% to about 40%, from about 20% to about 35%, from about 20% to about 30%, from about 20% to about 25%, from about 25% to about 50%, from about 25% to about 45%, from about 25% to about 40%, from about 25% to about 35%, from about 25% to about 30%, from about 30% to about 50%, from about 30% to about 45%, from about 30% to about 40%, from about 30% to about 35%, from about 35% to about 50%, from about 35% to about 45%, from about 35% to about 40%, from about 40% to about 50%, from about 40% to about 45%, or from about 45% to about 50%. In some embodiments, the antimicrobial silver salt can be present in amount by weight of the polymer membrane 10Q5 before crosslinking of about 20%, about 25%, about 30%, about 35%, about 40%, about 45%, or about 50%. In some embodiments, the antimicrobial silver salt can be present in amount by weight of the polymer membrane 10Q5 before crosslinking of about 35%.

### 6. Additional Antimicrobial Compounds

In some embodiments, the analyte sensor comprises at least one additional antimicrobial compound. In some embodiments, the analyte sensor comprises at least one additional antimicrobial compound in one or more components of the lower portion of the sensor tail substrate. In some embodiments, the analyte sensor comprises between 1 and 3, between 1 and 2, or between 2 and 3 additional antimicrobial compounds in one or more components of the lower portion of the sensor tail. In some embodiments, the analyte sensor comprises 1, 2, or 3 additional antimicrobial compounds in one or more components of the lower portion of the sensor tail.

The additional antimicrobial compounds for use in the present disclosure include all skin-compatible antimicrobial compounds, including those described hereinabove, regardless of their particular form (e.g., coating, particle, and the like). Examples of additional antimicrobial compounds, can include, but are not limited to, iodine, povidone-iodine, chlorhexidine, amphotericin B, bacitracin, colistin, gramicidin, gramicidin S, ritipenem, acediasulfone, acetosulfone, bambermycin(s), brodimoprim, butirosin, capreomycin, carbomycin, dapsone, diathymosulfone, enviomycin, glucosulfone solasulfone, leucomycin(s), lucensomycin, micronomicin, mupirocin, p-sulfanilylbenzylamine, pipemidic acid, polymyxin, primycin, ristocetin, rosaramycin, salazosulfadimidine, succisulfone, sulfachrysoidine, sulfaloxic acid, sulfamidochrysoidine, sulfanilic acid, sulfoxone, teicoplanin, tetroxoprim, thiazolsulfone, thiostrepton, trimethoprim, trospectomycin, tuberactinomycin, vancomycin, azaserine, candicidin(s), chlorphenesin, dermostatin(s), filipin, fungichromin, nystatin, oligomycin(s), perimycin A, tubercidin, carbapenems (e.g., faropenem, imipenem, panipenem, biapenem, meropenem, doripenem, ertapenem, and the like), cephalosporins (e.g., cefoxitin, cefotaxime, cefepime, nitrocefin, cefpirome, ceftobiprole, ceftazidime, ceftriaxone, cefadroxil, cefamandole, cefatrizine, cefbuperazone, cefclidin, cefdinir, cefditoren, cefetamet, cefixime, cefinenoxime, cefminox, cefodizime, cefonicid, cefoperazone, ceforanide, cefotetan, cefotiam, cefozopran, cefpimizole, cefpiramide, cefprozil, cefroxadine, cefteram, ceftibuten, cefuzonam, cephalexin, cephaloglycin, cephalosporin C, cephradine, flomoxef, moxalactam, and the like), monocyclic beta-lactams (e.g., aztreonam, tigemonam, carumonam, BAL19764, nocardicin, BAL30072, and the like), oxazolidinones (e.g., linezolid, sutezolid, and the like), penicillins (e.g., aminopenicillanic acid, benzylpenicillin, ampicillin, amoxicillin, carbenicillin, ticarcillin, oxacillin, azlocillin, dicloxacillin, piperacillin, apalcillin, temocillin, aspoxicillin, cyclacillin, epicillin, hetacillin, quinacillin, and the like), quinolones (e.g., nalidixic acid, norfloxacin, enoxacin, ciprofloxacin, enrofloxacin, levofloxacin, fleroxacin, clinafloxacin, grepafloxacin, lomefloxacin, nadifloxacin, pazufloxacin, temafloxacin, tosufloxacin, trovafloxacin, ciproflaxacin, ofloxacin, pefloxacin, rosoxacin, amifloxacin, temafloaxcin, lomefloxacin, sparfloxacin, and the like), tetracyclins (e.g., tetracycline, minocycline, tigecycline, apicycline, chlortetracycline, clomocycline, demeclocycline, doxycycline, guamecycline, lymecycline, meclocycline, methacycline, oxytetracycline, pipacycline, rolitetracycline, sancycline, and the like), aminoglycosides (e.g., amikacin, and the like), beta-lactamase inhibitors (e.g., clavulanic acid, sulbactam, avibactam, tazobactam, BAL29880, and the like), aminonucleosides (e.g., puromycin, and the like), aminoglycosides (e.g., apramycin, a kanamycin, isepamicin, a fortimicin, a gentamicin, neomycin, netilmicin, streptomycin, spectinomycin, arbekacin, dibekacin, dihydrostreptomycin, paromomycin, ribostamycin, sisomicin, tobramycin, and the like), anthracyclines (e.g., doxorubicin, daunorubicin, and the like), pimaricins (e.g., natamycin, and the like), sulfanilamides (e.g., 4,4′-sulfinyldianiline, 2-p-sulfanilyanilinoethanol, 4-sulfanilamidosalicylic acid, and the like), macrolides (e.g., mepartricin, spiramycin, clarithromycin, dirithromycin, erythromycin, josamycin, a midecamycin, oleandomycin, rokitamycin, roxithromycin, and the like), peptidyl transferase amphenicols (e.g., azidamfenicol, azithromycin, chloramphenicol, thiamphenicol, and the like), lincosamides (e.g., clindamycin, lincomycin, and the like), ansamycins (e.g., rifamycin SV, rifampin, rifapentine, rifaximin, rifamide, and the like), and the like, and any combinations thereof.

In some embodiments, metal-based additional antimicrobial compounds can be particularly useful for imparting the antimicrobial qualities to the sensor tail components (e.g., sensor substrate, electrode(s), sensing layer(s), non-electrochemical functional layer). These metal-based antimicrobial compounds can be a metal ion, a metal oxide, metal salts, metal coordination compounds including chelates, and the like. Specific examples of suitable metal-based additional antimicrobial compounds can include, but are not limited to, copper, copper sulfate, cupric oxalate, magnetite, gold, gallium, platinum, palladium, titanium dioxide, zinc oxide, magnesium oxide, silicon dioxide, iron oxide, carbon dioxide, copper oxide, nitric oxide, carbon nanotubes, and the like (e.g., other antimicrobial heavy metal ions and/or metal oxides), any alloys thereof, any salts thereof, any coordination complexes and/or chelates thereof, any combination thereof, and any combination thereof in addition to one or more of the antimicrobial compounds described herein.

In some embodiments, the metal-based antimicrobials can be metal-containing nanoparticles, such as impregnated or made whole from the antimicrobial compound, including as non-limiting examples, any nanoparticles comprised of the metal-based antimicrobial compounds described herein, including as non-limiting examples, zinc oxide nanoparticles, iron oxide nanoparticles, copper nanoparticles, and the like.

The particular amount of additional antimicrobial compound provided to any one or more components of the sensor tail can depend on a number of factors including, but not limited to, the physiology and daily routine of a user, the component(s) of the sensor tail to which the antimicrobial quality can be imparted, the particular antimicrobial mechanism and compound selected, and the like. In nonlimiting embodiments, the antimicrobial quality (or qualities) can be imparted by incorporation of one or more additional antimicrobial compounds in an amount of about 0.1% to about 50% by weight of the antimicrobial carrier (e.g., membrane, sensing element, non-electrochemical functional layer, coating, encapsulant, and the like) or surface area to which it can be applied (e.g., lower portion of the sensor tail), encompassing any value or subset therebetween, depending broadly on at least one or more of the factors provided above. In some embodiments, the additional antimicrobial compounds are present in an amount of about 0.1% to about 50% by weight of the membrane polymer before crosslinking, such as in the range of about 20% to about 40%, or about 30% to about 40%, encompassing any value and subset therebetween. It can be understood that in some instances the additional antimicrobial compound forms the entirety of some of the sensor tail components (e.g., the non-electrochemical functional layer) in some cases, without departing from the scope of the present disclosure. Moreover, it can be understood that the ideal additional antimicrobial compound amount will be based on ensuring adequate antimicrobial protection without compromising the functionality of the analyte sensor.

It can be understood that the tissue interacting components of the sensor tail and the antimicrobial quality imparted thereto are to be biocompatible, such that the tissue of a typical user is not adversely effected, bearing in mind that various users will have different physiologies. Moreover, a component of the sensing element (e.g., enzyme such as glucose oxidase) for a particular desired analyte being measured can additionally provide an antimicrobial quality, without departing from the scope of the present disclosure.

### 7. Non-Electrochemical Functional Layer

It can be appreciated that typical analyte sensor tails do not include a non-electrochemical functional layer (separate and apart from the one or more electrodes of the analyte sensor), as described herein. The non-electrochemical functional layer(s) of the present disclosure are designed specifically to impart an antimicrobial quality to analyte sensors described in accordance with the embodiments described herein. The optional non-electrochemical functional layer can be one or more of a polymer, ceramic, metal, and/or metal salt material that is not in electrical communication with the sensor electronics. Accordingly, in some embodiments, the non-electrochemical functional layer can be separate from the one or more electrodes and does not interfere with the electrical communication characteristics of the analyte sensor. In some embodiments, the non-chemical functional layer can be disposed upon a portion of the sensor tail substrate below the sensing layer(s) and/or below the membrane.

The optional non-electrochemical functional layer can alternatively, or in addition to being disposed on at least the lower portion of the sensor tail substrate, be included in the analyte sensor as an independent layer and be located between any other layer or component. In such cases, the component(s) of the non-electrochemical functional layer can leach out to the exterior portions of the analyte sensor. In other embodiments, alone or in combination with other locations, the non-electrochemical functional layer can be disposed on any layer of the analyte sensor (e.g., the substrate and/or membrane), such that the non-electrochemical functional layer can be located at at least a portion of an outer feature which can be overcoated with membrane. Such an outer non-electrochemical functional layer can be discontiguous or contiguous and can expand the entirety of one or more outer faces or only a portion. The non-electrochemical functional layer accordingly can be at any location (or layer or face) of the analyte sensor described herein, provided that it can be electrically isolated from the working electrode(s) if it is composed of an otherwise electrochemically active composition within the functioning electrochemical range of the sensor. That is, the non-electrochemical functional layer is not intended to interfere with analyte measurement performed by the analyte sensor and should otherwise be isolated from the electrode(s) of the analyte sensor if it could interfere or otherwise be detrimental to any antimicrobial qualities imparted to the sensor, such as those described herein.

Such non-electrochemical functional layer can itself serve as the antimicrobial quality (e.g., composed of one or more antimicrobial compounds), and/or can be intermixed, chemically bound to, or otherwise serve as a carrier for an antimicrobial quality, as described hereinbelow and thereafter coated, for example, onto the sensor tail substrate (e.g., by spray coating, screen printing, 3D printing, dipping, and the like). In some embodiments, the non-electrical functional layer can be comprised of silver, copper oxalate, copper salts and oxides, cobalt, cobalt salts and oxides, nickel, nickel salts and oxides, zinc, zinc salts and oxides, zirconium, zirconium salts and oxides, molybdenum, molybdenum salts and oxides, lead, and lead salts and oxides, and the like, and any combination thereof.

While the non-electrochemical functional layer is preferably at least located at the lower portion of the sensor tail substrate, it can further be located anywhere along the length of the sensor tail (i.e., the upper and/or lower portion), without departing from the scope of the present disclosure. In some embodiments, the non-electrochemical functional layer can at least be located at the upper portion of the sensor tail substrate-particularly in embodiments where the membrane is not located in the upper portion of the sensor tail substrate. In some embodiments, the non-electrochemical functional layer can be located in the lower portion of the sensor tail substrate (i.e., the membrane covered area) and can also be located in the upper portion of the sensor tail substrate (i.e, the portion not covered by a membrane)—for example, as a single layer that transitions from the lower portion of the sensor tail to the upper portion of the sensor tail. As such, its antimicrobial quality and/or the antimicrobial quality imparted thereto in accordance with the embodiments of the present disclosure can provide antimicrobial protection to the entirety of the sensor tail and surrounding tissue, including the insertion site. Moreover, the non-electrochemical functional layer can be in the form of a single layer, or alternatively, can be an array of sensing areas or “spots,” without being bound by shape or size or composition. Further, the non-electrochemical functional layer can be preferably coated onto the sensor tail substrate, such as by screen print coating, 3D print coating, spray coating, dipping, and the like. It can be understood, however, that the non-electrochemical functional layer can be applied by other means, without departing from the scope of the present disclosure.

### 8. Enzymes

In some embodiments, the sensing layer can include one or more enzymes for detecting a particular analyte. The enzyme can catalyze a reaction that consumes an analyte of interest or produces a product that can be detectable by the analyte sensor. The analyte-responsive enzyme will be selected based on the analyte that can be detected (e.g., glucose, lactate, ketone, glutamate, pyruvate, creatinine, sarcosine, and/or alcohol (e.g., ethanol)). In some embodiments, the enzyme can be an oxidase enzyme or a dehydrogenase enzyme. Suitable examples of the analyte-responsive enzyme include glucose oxidase, glucose dehydrogenase, glutamate oxidase, lactate oxidase, lactate dehydrogenase, pyruvate oxidase, alcohol oxidase, xanthine oxidase, β-hydroxybutyrate dehydrogenase, 11β-hydroxysteroid dehydrogenase type 2 (11B-HSD-2), creatine amidohydrolase, sarcosine oxidase, nicotinamide adenine dinucleotide (NADH)-dependent oxidase, NADPH dehydrogenase, a flavin adenine dinucleotide (FAD)-dependent oxidase, a flavin mononucleotide (FMN)-dependent oxidase, diaphorase, catalase, and combinations thereof. In some embodiments, the enzyme can be glucose oxidase and/or glucose dehydrogenase to detect glucose. In some embodiments, the enzyme can be glutamate oxidase to detect glutamate. In some embodiments, the enzyme can be lactate oxidase and/or lactate dehydrogenase to detect lactate. In some embodiments, the enzyme can be pyruvate oxidase to detect pyruvate. In some embodiments, the enzymes are alcohol oxidase and xanthine oxidase to detect ethanol or other alcohols. In some embodiments, the enzyme can be β-hydroxybutyrate dehydrogenase to detect ketone. In some embodiments, the enzyme can be creatine amidohydrolase and/or sarcosine oxidase to detect creatine and/or sarcosine. If necessary, one or more cofactors can be included with the enzyme, which serves as a catalyst for the electron transfer. Suitable cofactors include, e.g., pyrroloquinoline quinone (PQQ), thiamine pyrophosphate (TPP), flavin adenine dinucleotide (FAD), nicotinamide adenine dinucleotide (NAD), and any combination thereof.

### 9. Redox Mediators

In some embodiments, an analyte sensor disclosed herein can include an electron transfer agent. For example, but not by way of limitation, one or more sensing layers of an analyte sensor can include an electron transfer agent. In some embodiments, an analyte sensor can include one sensing layer that includes an electron transfer agent and a second sensing layer that does not include an electron transfer agent. In some embodiments, the presence of an electron transfer agent in a sensing layer can depend on the enzyme or enzyme system used to detect the analyte and/or the composition of the working electrode. Alternatively, an analyte sensor can include two sensing layers, where both sensing layers include an electron transfer agent.

Suitable electron transfer agents can facilitate conveyance of electrons to the adjacent working electrode after an analyte undergoes an enzymatic oxidation-reduction reaction within the corresponding sensing layer, thereby generating a current that can be indicative of the presence of that particular analyte. The amount of current generated can be proportional to the quantity of analyte that is present. In some embodiments, suitable electron transfer agents can include electroreducible and electrooxidizable ions, complexes or molecules (e.g., quinones) having oxidation-reduction potentials that are a few hundred millivolts above or below the oxidation-reduction potential of the standard calomel electrode (SCE). In some embodiments, the redox mediators can include osmium complexes and other transition metal complexes, such as those described in U.S. Pat. Nos. 6,134,461 and 6,605,200, which are incorporated herein by reference in their entirety. Additional examples of suitable redox mediators include those described in U.S. Pat. Nos. 6,736,957, 7,501,053 and 7,754,093, the disclosures of each of which are also incorporated herein by reference in their entirety. Other examples of suitable redox mediators include metal compounds or complexes of ruthenium, osmium, iron (e.g., polyvinylferrocene or hexacyanoferrate), or cobalt, including metallocene compounds thereof, for example. Suitable ligands for the metal complexes can also include, for example, bidentate or higher denticity ligands such as, for example, bipyridine, biimidazole, phenanthroline, or pyridyl (imidazole). Other suitable bidentate ligands can include, for example, amino acids, oxalic acid, acetylacetone, diaminoalkanes, or o-diaminoarenes. Any combination of monodentate, bidentate, tridentate, tetradentate, or higher denticity ligands can be present in a metal complex, e.g., osmium complex, to achieve a full coordination sphere.

In some embodiments, electron transfer agents disclosed herein can comprise suitable functionality to promote covalent bonding to a polymer (also referred to herein as a polymeric backbone) within the sensing layers as discussed further below. For example, but not by way of limitation, an electron transfer agent for use in the present disclosure can include a polymer-bound electron transfer agent. Suitable non-limiting examples of polymer-bound electron transfer agents include those described in U.S. Pat. Nos. 8,444,834, 8,268,143 and 6,605,201, the disclosures of which are incorporated herein by reference in their entirety. In some embodiments, the electron transfer agent can be a bidentate osmium complex bound to a polymer described herein, e.g., a polymeric backbone described below. In some embodiments, the polymer-bound electron transfer agent shown in FIG. 3 of U.S. Pat. No. 8,444,834 can be used in a sensor of the present disclosure.

### 10. Polymeric Backbone

In some embodiments, one or more sensing layers for promoting analyte detection can include a polymer to which an enzyme and/or redox mediator is covalently bound. Any suitable polymeric backbone can be present in the sensing layer for facilitating detection of an analyte through covalent bonding of the enzyme and/or redox mediator thereto. Non-limiting examples of suitable polymers within the sensing layer include polyvinylpyridines, e.g., poly(4-vinylpyridine) and/or poly(2-vinylpyridine), and polyvinylimidazoles, e.g., poly(N-vinylimidazole) and poly(l-vinylimidazole), or a copolymer thereof, for example, in which quaternized pyridine groups serve as a point of attachment for the redox mediator or enzyme thereto. Illustrative copolymers that can be suitable for inclusion in the sensing layers include those containing monomer units such as styrene, acrylamide, methacrylamide, or acrylonitrile, for example. In some embodiments, polymers that can be present in a sensing layer include a polyurethane or a copolymer thereof, and/or polyvinylpyrrolidone. In some embodiments, polymers that can be present in the sensing layer include, but are not limited to, those described in U.S. Pat. No. 6,605,200, the contents of which are incorporated herein by reference in their entirety, such as poly(acrylic acid), styrene/maleic anhydride copolymer, methylvinylether/maleic anhydride copolymer (GANTREZ polymer), poly(vinylbenzylchloride), poly(allylamine), polylysine, poly(4-vinylpyridine) quaternized with carboxypentyl groups, and poly(sodium 4-styrene sulfonate). In some embodiments where the analyte sensor includes two sensing layers, the polymer within each sensing layer can be the same or different.

In some embodiments, the polymer can be polyvinylpyridine or a copolymer thereof. In some embodiments, the polymer can be a co-polymer of vinylpyridine and styrene.

In some embodiments, when an enzyme system with multiple enzymes is present in a given sensing layer, all of the multiple enzymes can be covalently bonded to the polymer. In some embodiments, only a subset of the multiple enzymes are covalently bonded to the polymer. For example, and not by the way of limitation, one or more enzymes within an enzyme system can be covalently bonded to the polymer and at least one enzyme can be non-covalently associated with the polymer, such that the non-covalently bonded enzyme can be physically retained within the polymer. In some embodiments, when a stabilizer is present in a sensing layer, one or more enzymes within the area can be covalently bonded to the stabilizer.

In some particular embodiments, covalent bonding of the one or more enzymes and/or redox mediators to the polymer and/or stabilizer in a given sensing layer can take place via crosslinking introduced by a suitable crosslinking agent. In some embodiments, crosslinking of the polymer to the one or more enzymes and/or redox mediators can reduce the occurrence of delamination of the enzyme compositions from the electrode. Suitable crosslinking agents can include one or more crosslinkable functionalities such as, but not limited to, vinyl, alkoxy, acetoxy, enoxy, oxime, amino, hydroxyl, cyano, halo, acrylate, epoxide, and isocyanato groups. In some embodiments, the crosslinking agent can comprise one or more, two or more, three or more, or four or more epoxide groups. For example, but not by way of limitation, a crosslinker for use in the present disclosure can include mono-, di-, tri- and tetra-ethylene oxides. In some embodiments, crosslinking agents for reaction with free amino groups in the enzyme (e.g., with the free side chain amine in lysine) can include crosslinking agents such as, for example, polyethylene glycol dibutyl ethers, polypropylene glycol dimethyl ethers, polyalkylene glycol allyl methyl ethers, polyethylene glycol diglycidyl ether (PEGDGE), or other polyepoxides, cyanuric chloride, N-hydroxysuccinimide, imidoesters, epichlorohydrin, or derivatized variants thereof. In some embodiments, the crosslinking agent can be PEGDGE, e.g., having an average molecular weight (Mn) from about 200 to 1,000, e.g., about 400. In some embodiments, the crosslinking agent can be PEGDGE 400. In some embodiments, the crosslinking agent can be glutaraldehyde. Suitable crosslinking agents for reaction with free carboxylic acid groups in the enzyme can include, for example, carbodiimides. In some embodiments, the crosslinking agent can be polyethylene glycol diglycidyl ether. In some embodiments, the crosslinking of the enzyme to the polymer can generally be intermolecular. In some embodiments, the crosslinking of the enzyme to the polymer can generally be intramolecular.

### 11. Mass Transport Limiting Membrane

In vivo analyte sensors for assaying glucose and other analytes can include a membrane disposed over at least a portion of the implanted portion of the analyte sensor. In some embodiments, the antimicrobial membrane can improve biocompatibility of the analyte sensor. In some embodiments, the antimicrobial membrane can be permeable or semi-permeable to an analyte of interest and limit the overall analyte flux to the active area of the analyte sensor (i.e., sensing layer(s)), such that the antimicrobial membrane also functions as a mass transport limiting membrane. Limiting analyte access to the sensing layer(s) of the analyte sensor with a mass transport limiting membrane can aid in avoiding sensor overload (saturation), thereby improving detection performance and accuracy. Such membranes can be highly specific toward limiting mass transport of a particular analyte, with other substances permeating through the membrane at significantly different rates, reducing background and interference signals from non-specific redox reactions with analyte molecules other than those of interests.

In some embodiments, the analyte sensors disclosed herein further include a mass transport limiting membrane permeable to an analyte that overcoats at least one sensing layer, e.g., a first sensing layer and/or a second sensing layer. In some embodiments, the mass transport limiting membrane overcoats one or more of the sensing layers of an analyte sensor.

In some embodiments, a mass transport limiting membrane overcoating a sensing layer can improve biocompatibility. A mass transport limiting membrane can act as a diffusion-limiting barrier to reduce the rate of mass transport of the analyte, e.g., glucose, an alcohol, a ketone, lactate or β-hydroxybutyrate, when the sensor is in use. For example, but not by way of limitation, limiting access of an analyte, e.g., an alcohol, to the sensing layer with a mass transport limiting membrane can aid in avoiding sensor overload (saturation), thereby improving detection performance and accuracy. In some embodiments, the mass transport limiting membrane can limit the flux of an analyte to the electrode in an electrochemical sensor so that the sensor is linearly responsive over a large range of analyte concentrations.

In some embodiments, a mass transport limiting can aid in retaining a cofactor (e.g., NAD or NADP) within a sensing layer while still permitting sufficient inward diffusion of the analyte to permit detection thereof.

In some embodiments, the mass transport limiting membrane can have a thickness, e.g., dry thickness, ranging from about 0.1 μm to about 1,000 μm, e.g., from about 1 μm to about 500 μm, from about 1 μm to about 100 μm, or from about 10 μm to about 100 μm. In some embodiments, the mass transport limiting membrane can have a thickness from about 0.1 μm to about 10 μm, e.g., from about 0.5 μm to about 10 μm, from about 1 μm to about 10 μm, from about 1 μm to about 5 μm, or from about 0.1 μm to about 5 μm.

In some embodiments, the mass transport limiting membrane can include two or more layers. In some embodiments, each layer can be formed by depositing a mass transport limiting membrane solution upon a surface, for example by dipping, and allowing the membrane solution to dry. In some embodiments, the sensor can be dipped in the mass transport limiting membrane solution more than once. For example, but not by way of limitation, a sensor (or working electrode) of the present disclosure can be dipped in an mass transport limiting membrane solution at least twice, at least three times, at least four times, or at least five times to obtain the desired mass transport limiting membrane thickness.

In some embodiments, the mass transport limiting membrane can be single-component (contain a single membrane polymer). Alternatively, the mass transport limiting membrane can be multi-component (contain two or more different membrane polymers). In some embodiments, the mass transport limiting membrane can include two or more layers, e.g., a bilayer or trilayer membrane. In some embodiments, each layer can comprise a different polymer or the same polymer at different concentrations or thicknesses.

In some embodiments, a mass transport limiting membrane can include a polyvinylpyridine (e.g., poly(4-vinylpyridine) or poly(4-vinylpyridine)), a polyvinylimidazole, a polyvinylpyridine copolymer (e.g., a copolymer of vinylpyridine and styrene), a polyacrylate, a polyurethane, a polyether urethane, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones and block copolymers thereof including, for example, di-block, tri-block, alternating, random and graft copolymers or a chemically related material and the like.

In some embodiments, the mass transport limiting membrane for use in the present disclosure, e.g., a single-component membrane, can include a polyvinylpyridine (e.g., poly(4-vinylpyridine) and/or poly(2-vinylpyridine)). In some embodiments, a mass transport limiting membrane for use in the present disclosure, e.g., a single-component membrane, can include poly(4-vinylpyridine). In some embodiments, a mass transport limiting membrane for use in the present disclosure, e.g., a single-component membrane, can include a copolymer of vinylpyridine and styrene. In some embodiments, the mass transport limiting membrane can comprise a polyvinylpyridine-co-styrene copolymer. For example, but not by way of limitation, a polyvinylpyridine-co-styrene copolymer for use in the present disclosure can include a polyvinylpyridine-co-styrene copolymer in which a portion of the pyridine nitrogen atoms were functionalized with a non-crosslinked polyethylene glycol tail and a portion of the pyridine nitrogen atoms were functionalized with an alkylsulfonic acid group. In some embodiments, a derivatized polyvinylpyridine-co-styrene copolymer for use as a membrane polymer can be the 10Q5 polymer as described in U.S. Pat. No. 8,761,857, the contents of which are incorporated by reference in their entirety. In some embodiments, the polyvinylpyridine-based polymer can have a molecular weight from about 50 Da to about 500 kDa.

In some embodiments, the mass transport limiting membrane can comprise polymers such as, but not limited to, poly(styrene co-maleic anhydride), dodecylamine and poly(propylene glycol)-block-polyethylene glycol)-block-poly(propylene glycol) (2-aminopropyl ether) crosslinked with poly(propylene glycol)-block-poly(ethylene glycol)-block-poly(propylene glycol) bis (2-aminopropyl ether); poly(N-isopropyl acrylamide); a copolymer of poly(ethylene oxide) and poly(propylene oxide); or a combination thereof.

In some embodiments, the mass transport limiting membrane can include a polyurethane membrane that includes both hydrophilic and hydrophobic regions. In some embodiments, a hydrophobic polymer component can be a polyurethane, a polyurethane urea or poly(ether-urethane-urea). In some embodiments, a polyurethane is a polymer produced by the condensation reaction of a diisocyanate and a difunctional hydroxyl-containing material. In some embodiments, a polyurethane urea is a polymer produced by the condensation reaction of a diisocyanate and a difunctional amine-containing material. In some embodiments, diisocyanates for use herein can include aliphatic diisocyanates, e.g., containing from about 4 to about 8 methylene units, or diisocyanates containing cycloaliphatic moieties. Additional non-limiting examples of polymers that can be used for the generation of a mass transport limiting membrane of the presently disclosed sensor include vinyl polymers, polyethers, polyesters, polyamides, inorganic polymers (e.g., polysiloxanes and polycarbosiloxanes), natural polymers (e.g., cellulosic and protein based materials) and mixtures (e.g., admixtures or layered structures) or combinations thereof. In some embodiments, the hydrophilic polymer component can be polyethylene oxide and/or polyethylene glycol. In some embodiments, the hydrophilic polymer component can be a polyurethane copolymer. In some embodiments, the mass transport limiting membrane can include a silicone polymer/hydrophobic-hydrophilic polymer blend. In some embodiments, the hydrophobic-hydrophilic polymer for use in the blend can be any suitable hydrophobic-hydrophilic polymer such as, but not limited to, polyvinylpyrrolidone, polyhydroxyethyl methacrylate, polyvinylalcohol, polyacrylic acid, polyethers such as polyethylene glycol or polypropylene oxide, and copolymers thereof, including, for example, di-block, tri-block, alternating, random, comb, star, dendritic, and graft copolymers. In some embodiments, the hydrophobic-hydrophilic polymer can be a copolymer of poly(ethylene oxide) (PEO) and poly(propylene oxide) (PPO). Non-limiting examples of PEO and PPO copolymers include PEO-PPO diblock copolymers, PPO-PEO-PPO triblock copolymers, PEO-PPO-PEO triblock copolymers, alternating block copolymers of PEO-PPO, random copolymers of ethylene oxide and propylene oxide and blends thereof. In some embodiments, the copolymers can be substituted with hydroxy substituents.

In some embodiments, hydrophilic or hydrophobic modifiers can be used to “fine-tune” the permeability of the resulting membrane to an analyte of interest. In some embodiments, hydrophilic modifiers such as poly(ethylene) glycol, hydroxyl or polyhydroxyl modifiers and the like, and any combinations thereof, can be used to enhance the biocompatibility of the polymer or the resulting mass transport limiting membrane.

In some embodiments where multiple sensing layers are present, a separate mass transport limiting membrane can overcoat each sensing layer. For example, but not by way of limitation, a mass transport limiting membrane can be disposed on the first sensing layer, e.g., an alcohol-responsive sensing layer, and a separate, second mass transport limiting membrane can overcoat the second sensing layer, e.g., a glucose-responsive sensing layer. In some embodiments, the two mass transport limiting membranes can be spatially separated and do not overlap each other. In some embodiments, the first mass transport limiting membrane does not overlap the second mass transport limiting membrane and the second mass transport limiting membrane does not overlap the first mass transport limiting membrane. In some embodiments, the first mass transport limiting membrane comprises different polymers than the second mass transport limiting membrane. Alternatively, the first mass transport limiting membrane comprises the same polymers as the second mass transport limiting membrane. In some embodiments, the first mass transport limiting membrane comprises the same polymers as the second mass transport limiting membrane but comprises different crosslinking agents.

In some embodiments, polydimethylsiloxane (PDMS) can be incorporated in any of the mass transport limiting membranes disclosed herein.

In some embodiments when a first sensing layer and a second sensing layer configured for assaying different analytes are disposed on separate working electrodes, the mass transport limiting membrane can have differing permeability values for the first analyte and the second analyte. For example, but not by way of limitation, the mass transport limiting membrane overcoating at least one of the sensing layers can include an admixture of a first membrane polymer and a second membrane polymer or a bilayer of the first membrane polymer and the second membrane polymer. A homogeneous membrane can overcoat the sensing layer not overcoated with the admixture or the bilayer, wherein the homogeneous membrane includes only one of the first membrane polymer or the second membrane polymer. Advantageously, the architectures of the analyte sensors disclosed herein readily allow a continuous membrane having a homogenous membrane portion to be disposed upon a first sensing layer and a multi-component membrane portion to be disposed upon a second sensing layer of the analyte sensors, thereby equalizing the permeability values for each analyte concurrently to afford improved sensitivity and detection accuracy. Continuous membrane deposition can take place through sequential dip coating operations in particular embodiments.

In some embodiments, the mass transport limiting membrane discussed above can be a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

In some embodiments, a membrane can be formed by crosslinking in situ a polymer, including those discussed above, modified with a zwitterionic moiety, a non-pyridine copolymer component, and optionally another moiety that is either hydrophilic or hydrophobic, and/or has other desirable properties, in a buffer solution (e.g., an alcohol-buffer solution). The modified polymer can be made from a precursor polymer containing heterocyclic nitrogen groups. For example, a precursor polymer can be polyvinylpyridine or polyvinylimidazole. Optionally, hydrophilic or hydrophobic modifiers can be used to “fine-tune” the permeability of the resulting membrane to an analyte of interest. Optional hydrophilic modifiers, such as poly(ethylene glycol), hydroxyl or polyhydroxyl modifiers, and the like, and any combinations thereof, can be used to enhance the biocompatibility of the polymer or the resulting membrane.

In some embodiments, the membrane can comprise a compound including, but not limited to, poly(styrene-co-maleic anhydride), dodecylamine and poly(propylene glycol)-block-poly(ethylene glycol)-block-poly(propylene glycol) (2-aminopropyl ether) crosslinked with poly(propylene glycol)-block-poly(ethylene glycol)-block-poly(propylene glycol) bis (2-aminopropyl ether); poly(N-isopropyl acrylamide); a copolymer of poly(ethylene oxide) and poly(propylene oxide); polyvinylpyridine; a derivative of polyvinylpyridine; polyvinylimidazole; a derivative of polyvinylimidazole; and the like; and any combination thereof. In some embodiments, the membrane can be comprised of a polyvinylpyridine-co-styrene polymer, in which a portion of the pyridine nitrogen atoms are functionalized with a non-crosslinked poly(ethylene glycol) tail and a portion of the pyridine nitrogen atoms are functionalized with an alkylsulfonic acid group (“Formula 1”).

In some embodiments, the mass transport limiting membrane can comprise a membrane polymer crosslinked with a crosslinking agent disclosed herein. In some embodiments where there are two mass transport limiting membranes, e.g., a first mass transport limiting membrane and a second mass transport limiting membrane, each membrane can be crosslinked with a different crosslinking agent. For example, but not by way of limitation, the crosslinking agent can result in a membrane that is more restrictive to diffusion of certain compounds, e.g., analytes within the membrane, or less restrictive to diffusion of certain compounds, e.g., by affecting the size of the pores within the membrane. For example, but not by way of limitation, in a sensor that can be configured to detect alcohol and glucose, the mass transport limiting membrane overcoating the alcohol-responsive area can have a pore size that restricts the diffusion of compounds larger than alcohol, e.g., glucose, through the membrane.

In some embodiments, crosslinking agents for use in the present disclosure can include polyepoxides, carbodiimide, cyanuric chloride, triglycidyl glycerol, N-hydroxysuccinimide, imidoesters, epichlorohydrin or derivatized variants thereof. In some embodiments, a membrane polymer overcoating one or more sensing layers can be crosslinked with a branched crosslinker, e.g., which can decrease the amount of extractables obtainable from the mass transport limiting membrane. Non-limiting examples of a branched crosslinker include branched glycidyl ether crosslinkers, e.g., including branched glycidyl ether crosslinkers that include two or three or more crosslinkable groups. In some embodiments, the branched crosslinker can include two or more crosslinkable groups, such as polyethylene glycol diglycidyl ether. In some embodiments, the branched crosslinker can include three or more crosslinkable groups, such as polyethylene glycol tetraglycidyl ether. In some embodiments, the membrane polymer can include polyvinylpyridine or a copolymer of vinylpyridine and styrene crosslinked with a branched glycidyl ether crosslinker including two or three crosslinkable groups, such as polyethylene glycol tetraglycidyl ether or polyethylene glycol diglycidyl ether. In some embodiments, the epoxide groups of a polyepoxides, e.g., polyethylene glycol tetraglycidyl ether or polyethylene glycol diglycidyl ether, can form a covalent bond with pyridine or an imidazole via epoxide ring opening resulting in a hydroxyalkyl group bridging a body of the crosslinker to the heterocycle of the membrane polymer.

The membrane compounds described herein can further be crosslinked with one or more crosslinking agents, including those listed above with reference to the enzyme described herein. For example, suitable crosslinking agents can include, but are not limited to, polyethylene glycol diglycidylether (PEGDGE), glycerol triglycidyl ether (Gly3), polydimethylsiloxane diglycidylether (PDMS-DGE), or other polyepoxides, cyanuric chloride, N-hydroxysuccinimide, imidoesters, epichlorohydrin, or derivatized variants thereof, and any combination thereof. Branched versions with similar terminal chemistry are also suitable for the present disclosure. For example, in some embodiments, Formula 1 can be crosslinking with triglycidyl glycerol ether and/or PEGDGE and/or polydimethylsiloxane diglycidylether (PDMS-DGE).

A membrane can be formed in situ by applying an alcohol-buffer solution of a crosslinker and a modified polymer over the sensing layer(s) and any additional compounds included in the sensing layer(s) (e.g., electron transfer agent) and allowing the solution to cure for about one to two days or other appropriate time period. The crosslinker-polymer solution can be applied over the sensing layer(s) by placing a droplet or droplets of the membrane solution on at least the sensing layer(s) of the sensor tail, by dipping the sensor tail into the membrane solution, by spraying the membrane solution on the sensor, by heat pressing or melting the membrane in any sized layer (such as discrete or all encompassing) and either before or after singulation, vapor deposition of the membrane, powder coating of the membrane, and the like, and any combination thereof. In order to coat the distal and side edges of the sensor, the membrane material can be applied subsequent to application (e.g., singulation) of the sensor electronic precursors (e.g., electrodes). In some embodiments, the analyte sensor can be dip-coated following electronic precursor application to apply one or more membranes. Alternatively, the analyte sensor could be slot-die coated wherein each side of the analyte sensor can be coated separately. A membrane applied in the above manner can have any of various functions including, but not limited to, mass transport limitation (i.e., reduction or elimination of the flux of one or more analytes and/or compounds that reach the sensing elements), biocompatibility enhancement, interferent reduction, and the like, and any combination thereof.

Generally, the thickness of the membrane can be controlled by the concentration of the membrane solution, by the number of droplets of the membrane solution applied, by the number of times the sensor can be dipped in the membrane solution, by the volume of membrane solution sprayed on the sensor, and the like, and by any combination of these factors. In some embodiments, the membrane described herein can have a thickness ranging from about 0.1 micrometers (pm) to about 1000 pm, encompassing any value and subset therebetween, such as from about 1 pm to and about 500 pm, or about 10 pm to about 100 pm. As stated above, the membrane can overlay one or more sensing elements, and in some embodiments, the sensing elements can have a thickness of from about 0.1 pm to about 10 pm, encompassing any value and subset therebetween. In some embodiments, a series of droplets can be applied atop one another to achieve the desired thickness of the sensing element and/or membrane, without substantially increasing the diameter of the applied droplets (i.e., maintaining the desired diameter or range thereof). Each single droplet for example can be applied and then allowed to cool or dry, followed by one or more additional droplets. Sensing elements and membrane can, but need not be, the same thickness throughout or composition throughout.

In some embodiments, the membrane composition for use as a mass transport limiting layer of the present disclosure can comprise polydimethylsiloxane (PDMS), polydimethylsiloxane diglycidylether (PDMS-DGE), aminopropyl terminated polydimethylsiloxane, and the like, and any combination thereof for use as a leveling agent (e.g., for reducing the contact angle of the membrane composition or sensing layer(s) composition). Branched versions with similar terminal chemistry are also suitable for the present disclosure. Certain leveling agents can additionally be included, such as those found, for example, in U.S. Pat. No. 8,983,568, the disclosure of which is incorporated by reference herein in its entirety.

In some embodiments, the membrane can form one or more bonds with the sensing layer(s). As used herein, the term “bonds,” refers to any type of an interaction between atoms or molecules that allows chemical compounds to form associations with each other, such as, but not limited to, covalent bonds, ionic bonds, dipole-dipole interactions, hydrogen bonds, London dispersion forces, and the like, and any combination thereof. For example, in situ polymerization of the membrane can form crosslinks between the polymers of the membrane and the polymers in the sensing layer(s). In some embodiments, crosslinking of the membrane to the sensing layer(s) facilitates a reduction in the occurrence of delamination of the membrane from the sensor.

In some embodiments, the crosslinking agent can be polyethylene glycol diglycidyl ether (PEGDGE). In some embodiments, the PEGDGE used to promote crosslinking (e.g., intermolecular crosslinking) between two or more membrane polymer backbones can exhibit a broad range of suitable molecular weights. In some embodiments, the molecular weight of the PEGDGE can range from about 100 g/mol to about 5,000 g/mol. The number of ethylene glycol repeat units in each arm of the PEGDGE can be the same or different, and can typically vary over a range within a given sample to afford an average molecular weight. In some embodiments, the PEGDGE for use in the present disclosure has an average molecular weight (Mn) from about 200 to 1,000, e.g., about 400. In some embodiments, the crosslinking agent can be PEGDGE 400.

In some embodiments, the polyethylene glycol tetraglycidyl ether used to promote crosslinking (e.g., intermolecular crosslinking) between two or more membrane polymer backbones can exhibit a broad range of suitable molecular weights. Up to four polymer backbones can be crosslinked with a single molecule of the polyethylene glycol tetraglycidyl ether crosslinker. The number of ethylene glycol repeat units in each arm of the polyethylene glycol tetraglycidyl ether can be the same or different, and can typically vary over a range within a given sample to afford an average molecular weight.

### 12. Interference Domain

In some embodiments, the sensor of the present disclosure, e.g., sensor tail, can further comprise an interference domain. In some embodiments, the interference domain can include a polymer domain that restricts the flow of one or more interferents, e.g., to the surface of the working electrode. In some embodiments, the interference domain can function as a molecular sieve that allows analytes and other substances that are to be measured by the working electrode to pass through, while preventing passage of other substances such as interferents. In some embodiments, the interferents can affect the signal obtained at the working electrode. Non-limiting examples of interferents include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea, and uric acid.

In some embodiments, the interference domain can be located between the working electrode and one or more sensing layers, e.g., alcohol-responsive sensing layer. In some embodiments, non-limiting examples of polymers that can be used in the interference domain include polyurethanes, polymers having pendant ionic groups, and polymers having controlled pore size. In some embodiments, the interference domain can be formed from one or more cellulosic derivatives. Non-limiting examples of cellulosic derivatives include polymers such as cellulose acetate, cellulose acetate butyrate, 2-hydroxyethyl cellulose, cellulose acetate phthalate, cellulose acetate propionate, cellulose acetate trimellitate, and the like.

In some embodiments, the interference domain can be part of the mass transport limiting membrane and not a separate membrane.

In some embodiments, the interference domain can include a thin, hydrophobic membrane that can be non-swellable and restricts diffusion of high molecular weight species. For example, but not by way of limitation, the interference domain can be permeable to relatively low molecular weight substances, such as hydrogen peroxide, while restricting the passage of higher molecular weight substances, such as ketones, glucose, acetaminophen, and/or ascorbic acid.

In some embodiments, the interference domain can be deposited directly onto the working electrode, e.g., onto the surface of the working electrode. In some embodiments, the interference domain has a thickness, e.g., dry thickness, ranging from about 0.1 μm to about 1,000 μm, e.g., from about 1 μm to about 500 μm, from about 1 μm to about 100 μm, or from about 10 μm to about 100 μm. In some embodiments, the interference domain can have a thickness from about 0.1 μm to about 10 μm, e.g., from about 0.5 μm to about 10 μm, from about 1 μm to about 10 μm, from about 1 μm to about 5 μm, or from about 0.1 μm to about 5 μm. In some embodiments, the sensor can be dipped in the interference domain solution more than once. For example, but not by way of limitation, a sensor (or working electrode) of the present disclosure can be dipped in an interference domain solution at least once, at least twice, at least three times, at least four times, or at least five times to obtain the desired interference domain thickness.

### 13. Manufacturing

The present disclosure further provides methods for manufacturing the presently disclosed analyte sensors that includes one or more sensing layers and one or more working electrodes.

In some embodiments, the method includes depositing an enzyme composition on a working electrode. In some embodiments, the enzyme composition can include one or more additional enzymes, e.g., diaphorase, a crosslinking agent, e.g., polyethylene glycol diglycidyl ether, and/or a redox mediator. In some embodiments, the enzyme composition can be deposited onto the surface of a working electrode as one large application which covers the desired portion of the working electrode or in the form of an array of a plurality of enzyme compositions, e.g., spaced apart from each other, to generate one or more sensing layers for detecting one or more analytes. In some embodiments, the method can further include curing the enzyme composition.

In some embodiments, the layers can be prepared as solutions that dry or cure to solidify after deposition. Therefore, in some embodiments, all layers can be deposited in an automated fashion using small-volume liquid handling or similar techniques for high-throughput sensor fabrication.

In some embodiments, the method can further include adding a membrane composition on top of the cured sensing layer and/or around the entire sensor. In some embodiments, the membrane composition can be a mass transport limiting membrane. In some embodiments, the method can include curing the membrane composition.

In some embodiments, the present disclosure provides a method of preparing an analyte sensor overcoated with a membrane comprising:
(a) mixing a silver salt having a particle size of ≤60 μm and at least one membrane polymer to form a silver salt dispersion, wherein the dispersion comprises 20% to 50% by weight of the silver salt;
(b) dipping an analyte sensor into the silver salt dispersion to overcoat the analyte sensor with the silver salt dispersion;
(c) curing the analyte sensor; and
(d) baking the analyte sensor.

In some embodiments, the present disclosure provides a method of preparing an analyte sensor overcoated with a membrane comprising:
(a) mixing a silver salt having a maximum particle diameter of ≤60 μm and at least one membrane polymer to form a silver salt dispersion, wherein the dispersion comprises 20% to 50% by weight of the silver salt;
(b) dipping an analyte sensor into the silver salt dispersion to overcoat the analyte sensor with the silver salt dispersion;
(c) curing the analyte sensor; and
(d) baking the analyte sensor.

In some embodiments, the curing of the analyte sensor overcoated with a membrane can be at a temperature from about 20° C. to about 50° C. In some embodiments, the curing of the analyte sensor overcoated with a membrane can be at a temperature from about 20° C. to about 50° C., from about 20° C. and about 40° C., from about 20° C. and about 30° C., from about 20° C. and about 25° C., from about 25° C. to about 50° C., from about 25° C. and about 40° C., from about 25° C. and about 30° C., from about 30° C. to about 50° C., from about 30° C. and about 40° C., or from about 40° C. and about 50° C. In some embodiments, the curing of the analyte sensor overcoated with a membrane can be at a temperature of about 25° C.

In some embodiments, the curing of the analyte sensor overcoated with a membrane can be for a length of time from about 2 hours to about 48 hours. In some embodiments, the curing of the analyte sensor overcoated with a membrane can be for a length of time from about 2 hours to about 48 hours, from about 2 hours and about 36 hours, from about 2 hours and about 24 hours, from about 2 hours and about 12 hours, from about 2 hours and about 6 hours, from about 6 hours to about 48 hours, from about 6 hours and about 36 hours, from about 6 hours and about 24 hours, from about 6 hours and about 12 hours, from about 12 hours to about 48 hours, from about 12 hours and about 36 hours, from about 12 hours and about 24 hours, from about 24 hours to about 48 hours, from about 24 hours and about 36 hours, or from about 36 hours and about 48 hours. In some embodiments, the curing of the analyte sensor overcoated with a membrane can be for a length of time of about 24 hours.

In some embodiments, the curing of the analyte sensor overcoated with a membrane can be at a relative humidity of from about 50% to about 80%. In some embodiments, the curing of the analyte sensor overcoated with a membrane can be at a relative humidity from about 50% and about 80%, from about 50% and about 70%, from about 50% and about 60%, from about 50% to about 65%, from about 60% and about 80%, from about 60% and about 70%, from about 60% to about 65%, from about 65% and about 80%, from about 65% and about 70%, or from about 70% and about 80%. In some embodiments, the curing of the analyte sensor overcoated with a membrane can be at a relative humidity of about 60%.

In some embodiments, the baking of the analyte sensor overcoated with a membrane can be at a temperature from about 40° C. to about 80° C. In some embodiments, the baking of the analyte sensor overcoated with a membrane can be at a temperature from about 40° C. to about 80° C., from about 40° C. and about 60° C., from about 40° C. and about 50° C., from about 50° C. and about 80° C., from about 50° C. to about 60° C., or from about 60° C. to 80° C. In some embodiments, the baking of the analyte sensor overcoated with a membrane can be at a temperature of about 56° C.

In some embodiments, the baking of the analyte sensor overcoated with a membrane can be for a length of time from about 24 hours to about 96 hours. In some embodiments, the baking of the analyte sensor overcoated with a membrane can be for a length of time from about 24 hours to about 96 hours, from about 24 hours and about 72 hours, from about 24 hours and about 50 hours, from about 24 hours and about 48 hours, from about 24 hours and about 40 hours, from about 40 hours to about 96 hours, from about 40 hours and about 72 hours, from about 40 hours and about 50 hours, from about 40 hours and about 48 hours, from about 48 hours to about 96 hours, from about 48 hours and about 72 hours, from about 48 hours and about 50 hours, from about 50 hours to about 96 hours, from about 50 hours and about 72 hours, or from about 72 hours and about 96 hours. In some embodiments, the baking of the analyte sensor overcoated with a membrane can be for a length of time of about 48 hours.

### IV. Analyte Monitoring

The present disclosure further provides methods of using the analyte sensors disclosed herein to detect an analyte in vivo. In some embodiments, the present disclosure provides methods for detecting one or more analytes, e.g., one analyte or two analytes. For example, but not by way of limitation, the present disclosure provides methods for detecting one or more analytes including glucose, ketones, lactate, oxygen, hemoglobin A1C, albumin, alcohol, alkaline phosphatase, alanine transaminase, aspartate aminotransferase, bilirubin, blood, urea, nitrogen, calcium, carbon dioxide, chloride, creatinine, hematocrit, lactate, magnesium, oxygen, pH, phosphorus, potassium, sodium, total protein, and/or uric acid. In some embodiments, the analyte can be ketones, alcohol, glucose, and/or lactate. For example, but not by way of limitation, the present disclosure provides methods for detecting one or more ketones. In some embodiments, the present disclosure provides methods for detecting glucose. In some embodiments, the present disclosure provides methods for detecting creatinine. In some embodiments, the present disclosure provides methods for detecting lactate. In some embodiments, the present disclosure provides methods for detecting alcohol.

Generally, monitoring the in vivo concentration of an analyte in a fluid of the body of a subject includes inserting at least partially under a skin surface an in vivo analyte sensor as disclosed herein, contacting the monitored fluid (interstitial, blood, dermal, and the like) with the inserted sensor and generating a sensor signal at the working electrode. The presence and/or concentration of the analyte detected by the analyte sensor can be displayed, stored, forwarded, and/or otherwise processed. A variety of approaches can be employed to determine the concentration of analyte (e.g., glucose, an alcohol, a ketone, and/or lactate) with the disclosed sensors. In some embodiments, monitoring the concentration of analyte using the sensor signal can be performed by coulometric, amperometric, voltammetric, potentiometric, or any other convenient electrochemical detection technique.

In some embodiments, a method for detecting an analyte includes:
(i) providing an analyte sensor including:
a sensor tail substrate having a lower portion and configured for insertion into a tissue;
a first working electrode disposed on the lower portion of the sensor tail substrate;
a sensing layer disposed upon a surface of the first working electrode; and
a membrane disposed over at least the sensing layer;
wherein the membrane comprises 20% to 50% by weight of a silver salt (ii) applying a potential to the first working electrode;
(ii) obtaining a first signal at or above an oxidation-reduction potential of the sensing layer, the first signal being proportional to a concentration of analyte in a fluid contacting the sensing layer; and
(iii) correlating the first signal to the concentration of analyte in the fluid.

In some embodiments, the method of the present disclosure can further include detecting a second analyte by providing an analyte sensor that includes a second sensing layer and/or exposing an analyte sensor that includes a second sensing layer to a fluid comprising the first analyte and the second analyte. In some embodiments, the analyte sensor for use in a method for detecting a first analyte and a second analyte can further include a second working electrode; and a second sensing layer disposed upon a surface of the second working electrode and responsive to a second analyte differing from the first analyte, where the second sensing layer comprises a second polymer, at least one enzyme responsive to the second analyte covalently bonded to the second polymer and, optionally, a redox mediator covalently bonded to the second polymer; wherein a portion, e.g., second portion, of the mass transport limiting membrane overcoats the second sensing layer. Alternatively, the second sensing layer can be covered by a second mass transport limiting membrane that is separate and/or different than a mass transport limiting membrane that overcoats the first sensing layer. In some embodiments, at least one enzyme responsive to the second analyte comprises an enzyme system comprising multiple enzymes that are collectively responsive to the second analyte.

In some embodiments, the method further includes attaching an electronics unit to the skin of the patient, coupling conductive contacts of the electronics unit to contacts of the sensor, collecting data using the electronics unit regarding a level of analyte from signals generated by the sensor, and forwarding the collected data from electronics unit to a receiver unit, e.g., by RF. In some embodiments, the receiver unit can be a mobile telephone. In some embodiments, the mobile telephone includes an application related to the monitored analyte. In some embodiments, analyte information can be forwarded by RFID protocol, such as BLUETOOTH®, and the like.

In some embodiments, the analyte sensor can be positioned in a user for automatic analyte sensing, e.g., continuously or periodically. In some embodiments, the level of the analyte can be monitored over a time period ranging from seconds to minutes, hours, days, weeks or months. In some embodiments, the methods disclosed herein can be used to predict future levels of the analyte, based on the obtained information, such as but not limited to current analyte level at time zero, as well as the rate of change of the analyte concentration or amount.

In some embodiments, the present disclosure further provides for a method comprising diffusing an antimicrobial quality into a tissue from an analyte sensor. As described above, the analyte sensor includes a sensor tail having a lower portion and an upper portion and configured for insertion into the tissue; at least one electrode disposed on the lower portion of the sensor tail substrate; a sensing element disposed upon a surface of the electrode; a membrane disposed over at least the sensing element; and an optional non-electrochemical functional layer disposed at least on the lower portion of the sensor tail substrate. At least one or more of the lower portion of the sensor tail substrate, the at least one electrode, the sensing element, the membrane, and the optional non-electrochemical functional layer comprise an antimicrobial quality. The diffusing can be bolus diffusing, sustained delivery diffusing, or dynamic diffusing, as described in greater detail hereinbelow. As a result of such diffusion, microorganism infection of the tissue can be reduced or prevented, thereby maintaining the functionality of the analyte sensor measurements and lifetime (e.g., improving sensor longevity), as well as the health of the tissue. In some embodiments, the analyte sensor, as described above, detects one or more analytes, such as glucose in the bodily fluid from the tissue (e.g., interstitial fluid) with the sensing layer located upon the sensor tail.

As described above, when positioning an analyte sensor onto the skin of a user, a needle or other introducer can be used to puncture the skin and allow transcutaneous implantation through the dermal region. Implantation can extend only to the dermis, or through to the subcutis. Accordingly, a transdermal skin wound can be created in order for the sensor to undergo positioning for analyte monitoring, wherein the sensor tail can be positioned trans- or subcutaneously into the skin through the wound (also referred to as the “insertion site”). Generally, an analyte sensor can be worn for a prolonged period of time, such as 7 days, 10 days, 14 days, 21 days, 30 days, or more. That is, the analyte sensor can be worn for a time period of less than 30 days or a period of 30 days or more. As such, the sensor and its various components can be exposed to various activities or atmospheres, including contact with the external environment, clothing, bath or shower water, rain or swimming water, social contact with other persons, and the like. While the adhesive pad assemblies of the present disclosure can provide complete protection from such exposure to the skin wound itself (insertion site), the possibility of existing microorganisms near the insertion site and/or migration of microorganisms from adjacent skin or areas, including the external environment, can still present a risk of incursion to the skin wound. Such exposure can create a rich environment for microorganism growth, which can be harmful to the user and/or can lead to altered functioning of the analyte sensor itself, such as causing a shortened life of the sensor and/or providing erroneous or altered data or perceived sensitivity and/or response times.

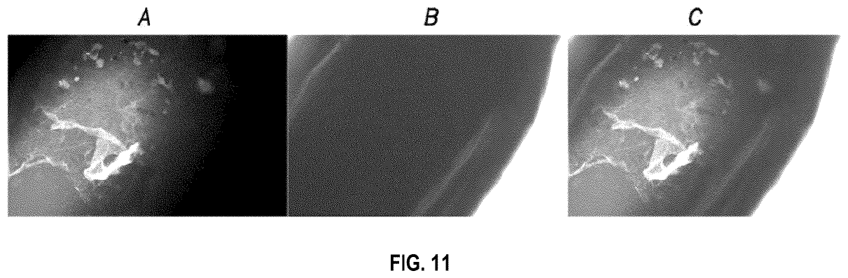

Referring now to FIG. 11, illustrated is a composite visible light and fluorescence image of a biofilm comprised of microorganisms formed on a sensor component of an analyte sensor having no antimicrobial quality. The biofilm on the sensor component is visualized at 10× OM magnification using fluorescence microscopy (labeled as “A”), light microscopy (labeled as “B”), and an overlay of the fluorescence and light microscopy images (labeled as “C”) after the analyte sensor was worn for 21 days. As shown, the biofilm is characterized by small, coccus-shaped (i.e., spherical or roughly spherical in shape) microorganisms. This biofilm can potentially migrate down the sensor tail of the analyte sensor toward the sensing region, which is located at a bottom portion of the sensor tail. As used herein, the term “sensing region,” and grammatical variants thereof, refers a portion of an analyte sensor comprising one or more components to facilitate measurement of one or more analytes (e.g., via electrochemical oxidation or reduction, or other sensing chemistry). The sensing region can include, for example, an analyte-specific reactant (e.g., enzyme or other chemical) that reacts with the analyte to produce a response at or near a working electrode. Accordingly, the sensing region includes at least the analyte-specific chemical and a working electrode. Other elements in the sensing region can include, but are not limited to, a membrane (e.g., a limited permeability membrane), other electrodes (e.g., reference and/or counter electrodes), an electron transfer agent to transfer electrons between the analyte and the working electrode (or other component), such as a suitable redox species, or both.

Contact of a biofilm, such as that depicted in FIG. 11, with or adjacent to the sensing region can affect various characteristics of the sensor, such as the accuracy of the sensor. If the biofilm reaches the sensing region, sensor sensitivity can be altered over the wear duration of the analyte sensor. Accordingly, protection of the sensing region from microorganisms can increase the life and functionality of the analyte sensor. Thus, protection of the insertion site from exposure to such microorganisms that can migrate to the implantation site can confer advantageous and unexpected operational benefits.

To facilitate a better understanding of the sensor tail architecture and sensing region, one or more embodiments thereof are shown in FIGS. 12-14. Placement of the antimicrobial quality onto (e.g., coating, functionalization, and the like) can be illustrated herein with reference to the sensor tail and sensing regions depicted in FIGS. 12-14. However, it can be understood that the antimicrobial quality can be provided on or about the upper portion of the sensor tail of the analyte sensors of the present disclosure to provide antimicrobial protection in the surrounding area and, in particular, to the sensing region where analyte measurements are determined, regardless of the configuration of said sensor tail or sensing region. The antimicrobial quality of the present disclosure is not provided to the sensing region (i.e., bottom portion of the sensor tail) according to the present disclosure.

For example, the sensor tail can have a plurality of electrodes located at a distal (bottom) end of the sensor tail (e.g., deeper into the subcutaneous space of a user's skin). One or more sensing regions can be associated with (e.g., coated upon or adjacent to) one or more of such electrodes, such as a working electrode. Such electrodes can be co-planar relative to each other, stacked relative to each other, helically wound about one another, incorporated with one or more insulating materials, and the like. The antimicrobial quality can then be provided in any location that imparts antimicrobial protection to the sensor, provided that it is not located at the bottom portion of the sensor tail and does not interfere with the operation of the sensing region or functionality of the sensor as a whole. That is, in some embodiments, the antimicrobial quality can be exterior to the body or only in contact dermally (i.e., passing through intact skin but not contacting the fatty subcutis layer under the skin) via the upper portion of the sensor tail.

FIG. 13 shows another embodiment of an analyte sensor in accordance with one or more embodiments of the present disclosure, and representing a variation of the sensor of FIGS. 12 and 14. Referring to FIG. 13, shown is an implantable (e.g., subcutaneous or transcutaneous) sensing region 1320 according to one or more embodiments of the present disclosure including a working electrode 1322. Proximal end 1340 is configured to be connected to various electrical connections for transmitting the output signals of the sensing region 1320. Collectively, the spans between the distal end 1325 and the proximal end 1340 form the sensor tail. Sensing region 1320 encompasses a bottom portion of the sensor tail. As depicted, sensing region 1320 comprises a rounded tip, but other tip shapes can alternately be present to facilitate insertion into a user's skin.

Additionally, in one or more embodiments, sensing region 1320 can include a reference electrode, a counter electrode, or counter-reference electrodes, such as those shown in FIGS. 12 and 14. Alternative electrode configurations can be employed without departing from the scope of the present disclosure. Sensing region 1320 can include one or more discrete sensing spots 1331, for example, in an area, such as area 1327.

### V. Sensor Testing

The presently disclosed analyte sensors were tested for their biocompatibility as medical devices.

Example 7 presents the results of a minimal essential media elution test. This test was conducted in accordance with ISO 10993-5:2009 Biological Evaluation of Medical Devices, Part 5—Tests for In Vitro Cytotoxicity.

ISO 10993-5 sets out a scheme for testing which requires decisions to be made by selecting from a series of tests. There are three categories of tests: an extract test, a direct contact test, and an indirect contact test. The choice of one or more of these categories of tests depends upon the nature of the sample to be tested, the potential site of use, and the nature of the use. The following categories for evaluation should be considered: (1) assessment of cell damage by morphological means; (2) measurement of cell damage; (3) measurement of cell growth; and (4) measurement of specific aspects of cellular metabolism.

A test on extracts is a test described in ISO 10993-5 which allows qualitative and/or quantitative assessment of cytotoxicity. Extraction conditions should attempt to simulate or exaggerate the clinical use conditions so as to determine the potential toxicological hazard without causing significant changes in the test sample, such as fusion, melting, or any alteration in the chemical structure, unless this is expected during clinical application. The final device should not be washed prior to extraction. The following steps are recommended to prepare liquid extracts of material for testing: (1) obtain a extract from the test sample (as well as from blanks and controls) using one of the following extraction vehicles: (a) culture medium with serum; (b) physiological saline solution; and/or (c) other suitable vehicle—culture medium with serum is the preferred extraction vehicle; (2) perform the extraction in sterile, chemically inert, closed containers using aseptic techniques (in accordance with ISO 10993-12); (3) conduct the extraction under one of the prescribed conditions: 37 (±1° C.) for 24 (±2) hours, 50 (±2)° C. for 72 (±2) hours, 70 (±2° C.) for 24 (±2) hours, or 121 (±2° C.) for 1 (±0.2) hours. If the extract is filtered, centrifuged, or processed by other methods before being applied to the cells, the details should be recorded along with a rationale for the additional steps. In some embodiments, the culture medium used in step (1) can be glucose-free minimal essential media. In some embodiments, the liquid extracts obtained from step (3) can be supplemented with 5% bovine serum post extraction.

After liquid extracts are obtained, the following steps are recommended to test the liquid extracts: (1) pipette an aliquot of a continuously stirred cell suspension into each of a sufficient number of vessels—with even distribution of cells over the surface of each vessel; (2) incubate the cultures at 37 (±1)° C. in air (with or without CO2) as appropriate for the chosen buffer system—the test should be performed on a subconfluent monolayer or on freshly suspended cells with a low cell density; (3) the subconfluency and the morphology of the cultures should be verified by microscope; (4) the test should be performed on either the original extract and/or the original extract and a dilution series of the extracts using the extract vehicle as diluent; (5) if a non-physiological extract is used (e.g., water), the extract should be tested at the highest physiologically compatible concentration after dilution in the culture medium; (6) aliquots of the blank as well as negative and positive controls should be added to replicate vessels; (7) incubate the vessels (test, blank, negative control, and negative control) at 37 (±1° C.) for an appropriate interval corresponding to the selected specific assay; and (8) incubate for at least 24 hours and determine the cytotoxic effects by qualitative and/or quantitative evaluation. In some embodiments, the cell suspension added in step (1) can comprise L-929 cells.

For qualitative evaluation, the cells should be examined microscopically using cytochemical staining if desired. Characteristics assessed include general morphology, vacuolization, detachment, cell lysis, and membrane integrity. Table 1 provides specific observations for qualitative morphological grading of cytotoxicity of extracts.

TABLE 1
Qualitative Morphological Grading of Cytotoxicity of Extracts

| Conditions of All Cultures | Reactivity | Grade |
|---|---|---|
| Discrete intracytoplasmic granules, no cell lysis, | None | 0 |
| no reduction of cell growth. | | |
| Not more than 20% of the cells are round, loosely | Slight | 1 |
| attached, and without intracytoplasmic granules, | | |
| or show changes in. morphology; occasional lysed | | |
| cells are present; only slight growth inhibition | | |
| observable. | | |
| Not more than 50% of the cells are round; devoid of | Mild | 2 |
| intracytoplasmic granules; no extensive cell lysis; | | |
| not more than 50% growth inhibition observable. | | |
| No more than 70% of the cell layers contain | Moderate | 3 |
| rounded cells or are lysed; cell layers are not | | |
| completely destroyed, but more than 50% growth | | |
| inhibition observable. | | |
| Nearly complete or complete destruction of the cell | Severe | 4 |
| layers. | | |

The overall assessment of the results should be carried out by a person capable of making informed decisions of the test data. Cytotoxicity data should be assessed in relation to other biocompatibility data and the intended use of the product. The interpretation of the results of the cytotoxicity test should take into account the classification of the device and the intended use of the product.

Based on the tests recommended by ISO 10993-5, a minimal essential media elution test was used to determine the cytotoxicity of extractable substances, described in detail in Example 7. This test allowed for both quantitative and qualitative assessment of cytotoxicity. An extract of the test sensor (the test article) was added to cell monolayers and incubated. The cell monolayers were then be examined and scored based on the degree of cellular destruction. The U.S. Pharmocopeia & National Formulary states that a test article meets its requirements and receives a passing score (PASS) if the reactivity grade is not greater than a grade of 2 (or mild reactivity). The ANSI/AAMI/ISO 10993-5 standard states that the achievement of a numerical grade of greater than 2 is considered a cytotoxic effect, or a failing score (FAIL).

As shown in Table 4 of Example 7, all test method acceptance criteria were met. Thus, as described in Example 7, the test sensor did not induce cytotoxicity as measured using the minimal essential media elution test according to ISO 10993-5.

Example 8 presents the results of a Guinea Pig Maximization Test. Testing was conducted in accordance with ISO 10993-10:2010 Biological Evaluation of Medical Devices, Part 10—Tests for Irritation and Skin Sensitization, Part 7.5, Guinea Pig Maximization Test and ISO 10993-12:2012 Biological Evaluation of Medical Devices, Part 12—Sample Preparation and Reference Materials.

The guinea pig maximization test is described in ISO 10993-10 and provides a test methodology for assessing skin sensitization based on application of single chemicals to a guinea pig. A range of dilutions of the test samples are applied topically to the flanks of guinea pigs. Dressings and patches are removed after 24 hours to assess the application sites for erythema and edema using the Magnusson and Kligman grading scale shown in Table 6 of Example 8.

The main test consists of three phases: (1) an intradermal induction phase in which intradermal injections are made to each animal at three injection sites; (2) a topical induction phase in which the test sample and a blank control are administered to all test and control animals by topical application into the intrascapular region of each animal; and (3) a challenge phase in which test sites of all test and control animals that were not treated in the topical induction phase are administered the test sample and a blank control. The challenge phase skin sites of the test and control animals are observed at 24 (±2) hours and 48 (±2) hours after removal of the dressings.

The overall assessment of the results should be carried out by a person capable of making informed decisions of the test data and that person should have no knowledge of the treatment in order to minimize bias in the evaluation of the results. Magnusson and Kligman grades of 1 or greater in the test group generally indicate sensitization when the control animals show grade of less than 1. If the control animals shows grades of 1 or greater, then the reactions of test animals which show a greater reaction than the most severe reaction in control animals are presumed to be due to sensitization. If uncertain of the results, rechallenge is recommended. The results of the tests are presented as the frequency of positive challenge results in test and control animals.

Based on the results described in Example 8, it was determined that the test analyte sensor did not cause skin irritation as measured by the Guinea pig maximization test of Magnusson and Kligman according to ISO 10993-10.

Example 9 presents the results of a hemolytic index test. Testing was conducted in accordance with ISO 10993-4:2017 Biological Evaluation of Medical Devices Part 4: Selection of Tests for Interaction with Blood.

ISO 10993-4 provides a structured test-selection system that is based on clinical concerns. The types of tests required by the standard depend on the blood contact category of the device or material (external communicating devices-blood path indirect, external communicating devices-circulating blood, and implant devices). For each contact category, primary (Level 1) and optional (Level 2) tests are recommended from the following list of general test categories: thrombosis, coagulation, platelets/platelet function, hematology, and immunology. Each test category represents a specific blood function.

Once a device's contact category is determined, one proceeds to the appropriate Level 1 table. In order to maximize hemocompatibility information about the device, one or more tests from each category included in the appropriate table should be performed.

All Level 1 tables include in vitro complement activation (immunology), hemolysis (hematology), and partial thromboplastin time (coagulation) tests. Such in vitro test methods are usually quicker and less costly than in vivo methods and do not require the use of animals. Complement activation is the most relevant immunology test for devices exposed to circulating blood. An increase in a downstream complement component over baseline levels indicates activation of the complement cascade. Acceptable complement activation limits have not been established, but comparative data are valuable. ASTM F 756, a standardized ASTM hemolysis test method, is available for determining the hemolytic potential of a device or material. These in vitro tests involve a quantitative measurement of plasma hemoglobin. An increase in plasma hemoglobin correlates with lysis of red blood cells, thereby indicating hemolytic activity of the material exposed to the cells. Such testing is frequently performed using rabbit blood. A device's effects on blood coagulation may be measured in vitro by determining the rate of clot formation or the partial thromboplastin time of plasma exposed to the biomaterial or device during an incubation period. The reaction of white blood cells to materials can also be used as an effective hematology test.

Thrombosis may be addressed by performing either an in vivo or ex vivo test. An evaluation of the thrombogenic potential of a device typically involves placing the device in a simulated clinical setting for a period of time, then removing the device and evaluating the extent of thrombus formation on or in it. The use of an appropriate control article is essential to the interpretation of results in these tests. The Lee White clotting-time test is also sometimes used to satisfy the requirement for a test in this category, but it is at best a gross screen because the dynamics of circulating blood are not present.

After completing the appropriate Level 1 tests, the device manufacturer must determine if any Level 2 tests are necessary. ISO 10993-4 does not indicate when such further testing is required. Because these tests are typically more complicated than Level 1 tests, and require specialized knowledge for their performance and interpretation, they are presumably most appropriate for situations when Level 1 test results indicate the device has an effect on a particular blood component. In such cases, Level 2 testing is usually a viable option for investigating the specific details of an observed effect. Level 2 tests also may be considered if an investigation into systemic effects, such as the immunotoxicity potential of a device, is warranted.

ASTM F 756 provides a protocol for the assessment of hemolytic properties of materials used in the fabrication of medical devices that will contact blood. Test and control specimens are to be prepared at a ratio of 3 cm² surface area to 1 mL of test blood solution. A minimum of 6 of each positive and negative control and each test sample should be prepared to use in a direct contract test and a test with the extract. Using the direct hemoglobin determination method applies the following steps: (1) prepare a hemoglobin calibration curve; and (2) prepare a standard curve from stock hemoglobin in 8 dilutions to accommodate a range of 1.4 to 0.03 mg/mL. The cyanmethemoglobin reagent diluent serves as a zero blank in a spectrometer. The absorbance of the samples are measured at 540 nm and a calibration curve is plotted using these values.

Based on the results described in Example 9, it was determined that the test analyte sensor is non-hemolytic according to ISO 10993-4 and ASTM F 756 (which provides a standard practice for assessment of hemolytic properties of materials). Example 10 presents the results of an Animal Intracutaneous (Intradermal) Irritation Test. Testing was conducted in accordance with ISO 10993-10:2010 Biological Evaluation of Medical Devices, Part 10—Tests for Irritation and Skin Sensitization, Part 6.4, Animal Intracutaneous (Intradermal) Irritation Test.

The animal intracutaneous (intradermal) irritation test is a test described in ISO 10993-10 and provides methodology for assessment of the potential of a medical device used as an implant to produce irritation following intradermal injection of extracts of the material. Test samples are prepared with polar or non-polar solvent, as applicable, and injected intracutaneously at five sites on one side of each test animal. Similarly, control samples are prepared with the same polar or non-polar solvent and injected intracutaneously at five sites on the contralateral side of each test animal. The appearance of each injection site is noted (1) immediately after injection; (2) 24 (±2) hours after injection; (3) 48 (±2) hours after injection; and (4) 72 (±2) hours after injection. The reaction of the tissue at each injection site and at each time interval is graded for erythema and edema according to the grading scale shown in Table 12 in Example 10.

After the 72 (±2) hours of grading is complete, all erythema and oedema grades at 24 (±2) hours, 48 (±2) hours, and 72 (±2) hours after injection are totaled separately for each test sample or control sample for each individual animal. To determine the overall mean score for each test sample and each corresponding control sample, the scores for all animals are added together and divided by the number of test animals. The final test sample score can be obtained by subtracting the score of the blank from the test sample score. If the final test score is 1.0 or less, the requirements of the test are met. If at any observation period the average reaction to the test sample is questionably greater than the average reaction of the control sample, the test should be repeated using the same number of additional animals.

Based on the results described in Example 10, it was determined that the test analyte sensor does not cause skin irritation as measured by the Animal Intracutaneous (Intradermal) Irritation Test according to ISO 10993-10.

Example 11 presents the results of a Bacterial Reverse Mutation Test. Testing was conducted in accordance with ISO 10993-3:2014 Biological Evaluation of Medical Devices, Part 3-Tests for Genotoxicity, Carcinogenicity and Reproductive Toxity and OECD Guidelines for Testing of Chemicals, Section 4, Test No. 471: Bacterial Reverse Mutation Test (OECD 471).

The Bacterial Reverse Mutation Test in OECD Guidelines for Testing of Chemicals, Section 4, Test No. 471: Bacterial Reverse Mutation Test (OECD 471), uses amino acid requiring strains of Salmonella typhimurium and Escherichia coli to detect point mutations involving substitution, addition, or deletion of one or a few base pairs. This test detects mutations that revert mutations present in the test strains and restore the functional capability of the bacteria to synthesize an amino acid. The revertant bacteria are detected by their ability to grow in the absence of the amino acid required by the test strain.

The following conditions are used to prepare bacterial samples: (1) fresh cultures are grown up to the late exponential or early stationary phase of growth (approximately 109 cells per mL); and (2) the culture temperature are kept at 37° C. At least five strains of bacteria are used which includes four strains of S. typhimurium that have been shown to be reliable and reproducibly responsive between laboratories. Established procedures for stock solution preparation, marker verification, and storage are used. An appropriate minimal agar and an overlay agar containing histidine and biotin or tryptophan, to allow for a few cell divisions, are used. Bacteria are exposed to the test substance both in the presence and absence of an appropriate metabolic activation system. Solid test substances are dissolved in appropriate solvents or vehicles and diluted if appropriate prior to treatment of the bacteria.

The following conditions are used to prepare the samples: (1) the recommended maximum test concentration for soluble non-cytotoxic substances is 5 mg/plate; (2) at least five different analyzable concentrations of the test substance are used with approximately half log intervals between test points for an initial experiment; and (3) concurrent strain-specific positive and negative (solvent or vehicle) controls, both with and without metabolic activation, are included in each assay and positive control concentrations that demonstrate effective performance of each assay should be selected. The following procedures are used for testing the samples: (1) determine whether to use the plate incorporation method or the pre-incubation method; (2) use triplicate plating at each dose level; (3) incubate all plates in a given assay at 37° C. for 48-72 hours; and (4) measure the number of revertant colonies per plate after the incubation period.

Results are presented as the number of revertant colonies per plate and the number of revertant colonies on both negative (solvent control and untreated control if used) and positive control plates. The results provided also include the individual plate counts, the number of revertant colonies per plate, and the standard deviation for the test substance and the positive and negative controls.

Based on the results described in Example 11, the test analyte sensor was not a potential mutagen as measured by the Bacterial Reverse Mutation Assay according to ISO 10993-3 and OECD 471.

Example 12 presents the results of an Acute Systemic Toxicity Test. The animal species, number, and route of test article administration were as recommended in ISO 10993-11:2017 Biological Evaluation of Medical Devices-Part 11: Tests for Systemic Toxicity, Part 5, Acute Systemic Toxicity.

The Acute Systemic Toxicity Test of ISO 10993-10 allows for assessment of the health hazards that are likely to arise from an acute exposure by the intended clinical route. Animals are treated by intravenous or intraperitoneal routes to screen solutions or test article extracts for potential toxic effects as a result of a single-dose systemic injection. The animal species, number, and route of test article administration are determined according to the intended clinical route. For the safety evaluation of the clinical route, animals are injected systemically with a dosage in an extraction solvent or vehicle determined by the clinical route to be employed. The observation period for an acute systemic toxicity study is at least 3 days. If tested for 3 days, the animals are observed for signs of toxicity at (1) 4 hours after injection; (2) 24 (±2) hours after injection; (3) 48 (±2) hours after injection; and (4) 72 (±2) hours after injection.

The evaluation criteria is based on the type of testing. If the testing is pharmacopoeia-type the following evaluation criteria are used: (1) if during the observation period, none of the animals treated with the test sample shows a significantly greater biological reactivity than animals treated with the control vehicle, the sample meets the test requirements; (2) using 5 animals, if 2 or more animals die, or if behavior such as convulsions or prostration occurs in 2 or more animals, or if a final (end of study) body weight loss greater than 10% occurs in 3 or more animals, the sample does not meet the test requirements; (3) if any animal treated with the test sample shows only slight signs of biological reactivity, and not more than 1 animal shows gross symptoms of biological reactivity and/or dies, repeat the test using 10 animals; and (4) on the repeat test prescribed by (3), if all 10 animals show no scientifically meaningful biological reactivity in excess of that observed for the control animals during the observation period, the sample meets the test requirements. There is an option to perform evaluations using more extensive methods including clinical and anatomic pathology, which may eliminate the need for a repeat test. A repeat of the evaluation is needed if equivocal differences are observed.

Includes the relationship between the dosage and the presence or absence and the incidence and severity of abnormalities, including behavioral and clinical abnormalities, gross lesions, body weight changes, effects on mortality, and any other observed effects in the evaluation.

Based on the results described in Example 12, the test analyte sensor does not cause acute systemic toxicity as measured by the Acute Systemic Toxicity Test according to ISO 10993-11.

Example 13 presents the results of an Intramuscular Injection Test. Testing was conducted in accordance with ISO 10993-6:2016 Biological Evaluation of Medical Devices, Part 6—Tests for Local Effects after Implantation.

Implanting a test article inside the body of a laboratory animal is the most direct means of evaluating a medical device material's potential effects on the surrounding living tissue. Samples are cut to size, if necessary; sterilized; and implanted aseptically. Then, after a period of time ranging from weeks to months, the implant sites are examined. Attention is focused entirely on local effects that occur in response to the presence of the test material that has been in intimate contact with living tissue. The selection of species, appropriate tissues for implantation, length of time implants should remain in place, implantation methods, and the evaluation of biological responses is selected based on the test article.

Although ISO 10993-6 mentions the use of mice, rats, and guinea pigs, the rabbit, because of its size and ease of handling, has long been the animal of choice for implant testing. In this model, test and control materials are cut into approximately 1×10-mm strips and placed in the lumens of 15-19-gauge needles. The samples are sterilized either before or after they are loaded into needles, but the method of sterilization is the same as that used on the final product to ensure that effects of the sterilization process on the material are taken into account.

After the rabbits are anesthetized and their skin shaved and prepared, four test samples are implanted in the paralumbar muscle on one side of the back and four plastic negative control (known nonreactive) samples are placed in the muscle on the opposite side. To evaluate materials used for short-term implants, local tissue response is assessed after 1, 4, and 12 weeks. For long-term use tests, intervals of 12, 26, 52, and 78 weeks are specified. Three or more animals are required for each test interval. Guinea pigs or other small rodents can be used in place of rabbits; however, in order to have enough sites to evaluate in small species, more than three animals per test interval are required. Recommended intervals can also vary somewhat if species other than rabbits are used.

At the end of each specified interval, each implant site is examined with the aid of a low-power lens and the size of the capsule surrounding the implant is recorded. Reactive materials generally produce a capsule that extends for 2 to 4 mm, while negative control materials generally produce no visible capsule at all. The implant is then removed (in most cases) and the tissues processed for histopathological examination. At the microscopic level, the nature and extent of cellular reaction to implants is evaluated and scored. Severe reactions are marked by the increased presence of inflammatory cells and the death of muscle cells surrounding the implant. Evaluations take into account the relative reactivity of the test and control materials.

Based on the results described in Example 13, the test analyte sensor does not cause intramuscular irritation as measured by the Intramuscular Implantation Test according to ISO 10993-6.

### VI. Exemplary Embodiments

(1) In some non-limiting embodiments, the presently disclosed subject matter provides for analyte sensors comprising:
a sensor tail substrate having a lower portion and configured for insertion into a tissue;
a first working electrode disposed on the lower portion of the sensor tail substrate;
a sensing layer disposed upon a surface of the first working electrode; and
a membrane disposed over at least the sensing layer;
wherein the membrane comprises 20% to 50% by weight of a silver salt with a particle size of ≤60 μm.
(2) The analyte sensor of (1), wherein the silver salt is uniformly distributed within the membrane.
(3) The analyte sensor of (1), wherein the silver salt is selected from the group consisting of silver chloride, silver-silver chloride, silver iodide, and combinations thereof.
(4) The analyte sensor of (1), wherein the silver salt is silver iodide.
(5) The analyte sensor of (4), wherein the silver iodide has a particle size of ≤53 μm.
(6) The analyte sensor of (4), wherein the silver iodide has a purity of at least 99%.
(7) The analyte sensor of (4), wherein the membrane comprises 30% to 40% by weight of silver iodide.
(8) The analyte sensor of (4), wherein the membrane comprises 35% by weight of silver iodide.
(9) The analyte sensor of (1), wherein the analyte sensor does not cause cytotoxicity according to ISO 10993-5.
(10) The analyte sensor of (1), wherein the analyte sensor does not cause skin irritation or skin sensitization according to ISO 10993-10.
(11) The analyte sensor of (1), wherein the analyte sensor does not cause intradermal irritation according to ISO 10993-10.
(12) The analyte sensor of (1), wherein the analyte sensor does not cause systemic toxicity according to ISO 10993-11.
(13) The analyte sensor of (1), wherein the analyte sensor is non-hemolytic according to ISO 10993-4 and ASTM F 756.
(14) The analyte sensor of (1), wherein the analyte sensor does not cause intramuscular irritation according to ISO 10993-6.
(15) The analyte sensor of (1), wherein the analyte sensor is not a potential mutagen according to ISO 10993-3 and OECD 471.
(16) In some non-limiting embodiments, the presently disclosed subject matter provides a method of inhibiting microorganism growth in an area in and around an insertion site comprising inserting an analyte sensor in a subject in need thereof comprising:
a first portion positionable above a surface of the skin;
a second portion positionable below the surface of the skin, wherein the second portion is in contact with bodily fluid and configured to measure signals indicative of analyte levels in the bodily fluid;
a first working electrode disposed on the second portion;
a sensing layer disposed upon a surface of the first working electrode; and
a membrane disposed over at least the sensing layer;
wherein the membrane comprises 20% to 50% by weight of a silver salt with a particle size of ≤60 μm, and wherein the analyte sensor does not induce cytotoxicity as measured according to ISO 10993-5.
(17) The method of (16), wherein the silver salt is silver iodide.
(18) The method of (17), wherein the silver iodide has a particle size of ≤53 μm.
(19) The method of (17), wherein the silver iodide has a purity of at least 99%.
(20) In some non-limiting embodiments, the presently disclosed subject matter provides a method of preparing an analyte sensor overcoated with a membrane comprising:
(a) mixing a silver salt having a particle size of ≤60 μm and at least one membrane polymer to form a silver salt dispersion, wherein the dispersion comprises 20% to 50% by weight of the silver salt;
(b) dipping an analyte sensor into the silver salt dispersion to overcoat the analyte sensor with the silver salt dispersion;
(c) curing the analyte sensor; and
(d) baking the analyte sensor.
(21) The method (20), wherein the silver salt is silver iodide.
(22) In some non-limiting embodiments, the presently disclosed subject matter provides an analyte sensor comprising:
a sensor tail substrate having a lower portion and configured for insertion into a tissue;
a first working electrode disposed on the lower portion of the sensor tail substrate;
a sensing layer disposed upon a surface of the first working electrode;
a membrane disposed over at least the sensing layer; and
a non-electrochemical functional layer comprising a silver salt;
wherein the analyte sensor does not induce cytotoxicity as measured according to ISO 10993-5.
(23) The analyte sensor of (22), wherein the silver salt is silver iodide with a particle size of ≤60 μm and a purity of at least 99%.
(24) The analyte sensor of (22), wherein the silver salt is silver-silver chloride.
(25) In some non-limiting embodiments, the presently disclosed subject matter provides an analyte sensor comprising:
a portion positionable into a tissue below the surface of the skin;
a first working electrode disposed on the portion positionable into a tissue;
a sensing layer disposed upon a surface of the first working electrode; and
a membrane disposed over at least the sensing layer;
wherein the membrane comprises 20% to 50% by weight of a silver salt with a maximum particle diameter of ≤60 μm.
(26) The analyte sensor of (25), wherein the silver salt is uniformly distributed within the membrane.
(27) The analyte sensor of (25), wherein the silver salt is selected from the group consisting of silver chloride, silver-silver chloride, silver iodide, and combinations thereof.
(28) The analyte sensor of (25), wherein the silver salt is silver iodide.
(29) The analyte sensor of (28), wherein the silver iodide has a maximum particle diameter of ≤53 μm.
(30) The analyte sensor of (28), wherein the silver iodide has a purity of at least 99%.
(31) The analyte sensor of (28), wherein the membrane comprises 30% to 40% by weight of silver iodide.
(32) The analyte sensor of (28), wherein the membrane comprises 35% by weight of silver iodide.
(33) The analyte sensor of (25), wherein the analyte sensor does not cause cytotoxicity according to ISO 10993-5.
(34) The analyte sensor of (25), wherein the analyte sensor does not cause skin irritation or skin sensitization according to ISO 10993-10.
(35) The analyte sensor of (25), wherein the analyte sensor does not cause intradermal irritation according to ISO 10993-10.
(36) The analyte sensor of (25), wherein the analyte sensor does not cause systemic toxicity according to ISO 10993-11.
(37) The analyte sensor of (25), wherein the analyte sensor is non-hemolytic according to ISO 10993-4 and ASTM F 756.
(38) The analyte sensor of (25), wherein the analyte sensor does not cause intramuscular irritation according to ISO 10993-6.
(39) The analyte sensor of (25), wherein the analyte sensor is not a potential mutagen according to ISO 10993-3 and OECD 471.
(40) In some non-limiting embodiments, the presently disclosed subject matter provides a method of inhibiting microorganism growth in an area in and around an insertion site comprising inserting an analyte sensor in a subject in need thereof comprising:
a first portion positionable above a surface of the skin;
a second portion positionable below the surface of the skin, wherein the second portion is in contact with bodily fluid and configured to measure signals indicative of analyte levels in the bodily fluid;
a first working electrode disposed on the second portion;
a sensing layer disposed upon a surface of the first working electrode; and
a membrane disposed over at least the sensing layer;
wherein the membrane comprises 20% to 50% by weight of a silver salt with a maximum particle diameter of ≤60 μm, and wherein the analyte sensor does not induce cytotoxicity as measured according to ISO 10993-5.
(41) The method of (40), wherein the silver salt is silver iodide.
(42) The method of (41), wherein the silver iodide has a maximum particle diameter of ≤53 μm.
(43) The method of (41), wherein the silver iodide has a purity of at least 99%.
(44) In some non-limiting embodiments, the presently disclosed subject matter provides a method of preparing an analyte sensor overcoated with a membrane comprising:
(a) mixing a silver salt having a maximum particle diameter of ≤60 μm and at least one membrane polymer to form a silver salt dispersion, wherein the dispersion comprises 20% to 50% by weight of the silver salt;
(b) dipping an analyte sensor into the silver salt dispersion to overcoat the analyte sensor with the silver salt dispersion;
(c) curing the analyte sensor; and
(d) baking the analyte sensor.
(45) The method (44), wherein the silver salt is silver iodide.
(46) In some non-limiting embodiments, the presently disclosed subject matter provides an analyte sensor comprising:
a portion positionable into a tissue below the surface of the skin;
a first working electrode disposed on the portion positionable into a tissue;
a sensing layer disposed upon a surface of the first working electrode;
a membrane disposed over at least the sensing layer; and
a non-electrochemical functional layer comprising a silver salt;
wherein the analyte sensor does not induce cytotoxicity as measured according to ISO 10993-5.
(47) The analyte sensor of (46), wherein the silver salt is silver iodide with a maximum particle diameter of ≤60 μm and a purity of at least 99%.
(48) The analyte sensor of (46), wherein the silver salt is silver-silver chloride.
(49) In some non-limiting embodiments, the presently disclosed subject matter provides an analyte sensor comprising:
a portion positionable into a tissue below the surface of the skin;
a first working electrode disposed on the portion positionable into a tissue;
a sensing layer disposed upon a surface of the first working electrode; and
a membrane disposed over at least the sensing layer;
wherein the membrane comprises 20% to 50% by weight of silver-silver chloride with a maximum particle diameter of ≤60 μm.
(50) The analyte sensor of (49), wherein the silver-silver chloride is uniformly distributed within the membrane.
(51) The analyte sensor of (49), wherein the silver-silver chloride has a maximum particle diameter of ≤53 μm.
(52) The analyte sensor of (49), wherein the silver-silver chloride has a purity of at least 99%.
(53) The analyte sensor of (49), wherein the membrane comprises 30% to 40% by weight of silver-silver chloride.
(54) The analyte sensor of (49), wherein the membrane comprises 35% by weight of silver-silver chloride.
(55) The analyte sensor of (49), wherein the analyte sensor does not cause cytotoxicity according to ISO 10993-5.
(56) The analyte sensor of (49), wherein the analyte sensor does not cause skin irritation or skin sensitization according to ISO 10993-10.
(57) The analyte sensor of (49), wherein the analyte sensor does not cause intradermal irritation according to ISO 10993-10.
(58) The analyte sensor of (49), wherein the analyte sensor does not cause systemic toxicity according to ISO 10993-11.
(59) The analyte sensor of (49), wherein the analyte sensor is non-hemolytic according to ISO 10993-4 and ASTM F 756.
(60) The analyte sensor of (49), wherein the analyte sensor does not cause intramuscular irritation according to ISO 10993-6.
(61) The analyte sensor of (49), wherein the analyte sensor is not a potential mutagen according to ISO 10993-3 and OECD 471.
(62) In some non-limiting embodiments, the presently disclosed subject matter provides a method of inhibiting microorganism growth in an area in and around an insertion site comprising inserting an analyte sensor in a subject in need thereof comprising:
a first portion positionable above a surface of the skin;
a second portion positionable below the surface of the skin, wherein the second portion is in contact with bodily fluid and configured to measure signals indicative of analyte levels in the bodily fluid;
a first working electrode disposed on the second portion;
a sensing layer disposed upon a surface of the first working electrode; and
a membrane disposed over at least the sensing layer;
wherein the membrane comprises 20% to 50% by weight of a silver-silver chloride with a maximum particle diameter of ≤60 μm, and wherein the analyte sensor does not induce cytotoxicity as measured according to ISO 10993-5.
(63) The method of (62), wherein the silver-silver chloride has a maximum particle diameter of ≤53 μm.
(64) The method of (62), wherein the silver-silver chloride has a purity of at least 99%.
(65) In some non-limiting embodiments, the presently disclosed subject matter provides a method of preparing an analyte sensor overcoated with a membrane comprising:
(a) mixing a silver-silver chloride having a maximum particle diameter of ≤60 μm and at least one membrane polymer to form a silver-silver chloride dispersion, wherein the dispersion comprises 20% to 50% by weight of the silver-silver chloride;
(b) dipping an analyte sensor into the silver-silver chloride dispersion to overcoat the analyte sensor with the silver-silver chloride dispersion;
(c) curing the analyte sensor; and
(d) baking the analyte sensor.
(66) In some non-limiting embodiments, the presently disclosed subject matter provides an analyte sensor comprising:
a portion positionable into a tissue below the surface of the skin;
a first working electrode disposed on the portion positionable into a tissue;
a sensing layer disposed upon a surface of the first working electrode;
a membrane disposed over at least the sensing layer; and
a non-electrochemical functional layer comprising a silver-silver chloride;
wherein the analyte sensor does not induce cytotoxicity as measured according to ISO 10993-5.
(67) The analyte sensor of (66), wherein the silver-silver chloride has a maximum particle diameter of ≤60 μm and a purity of at least 99%.

To facilitate a better understanding of the embodiments described herein, the following examples of various representative embodiments are given. In no way should the following examples be read to limit, or to define, the scope of the invention.

### EXAMPLES

The presently disclosed subject matter will be better understood by reference to the following Examples, which are provided as exemplary of the presently disclosed subject matter, and not by way of limitation.

### Comparative Example 1: Biofilm Observation and Signal Attenuation

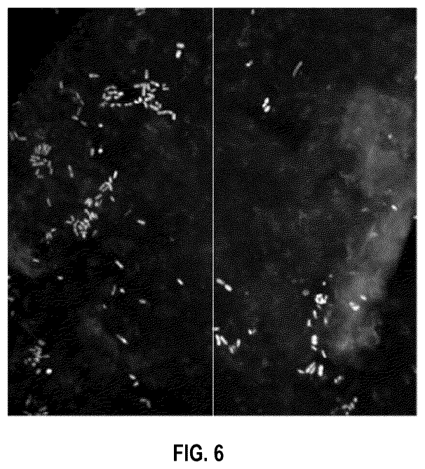

In this example from U.S. application Ser. No. 17/052,705, analyte sensors for measuring glucose were observed for potential late signal attenuation after formation of a biofilm on the surface of the lower portion of a sensor tail comprising the sensing element. As shown in FIG. 6, illustrated is a fluorescent micrograph image showing 4′,6-diamidino-2-phenylindole (DAPI) staining of a biofilm comprised of microorganisms formed on a sensor tail of an analyte sensor having no antimicrobial quality. As shown, the biofilm is characterized by small rod-shaped (bacilli) and spherical-shaped (rod) microorganisms.

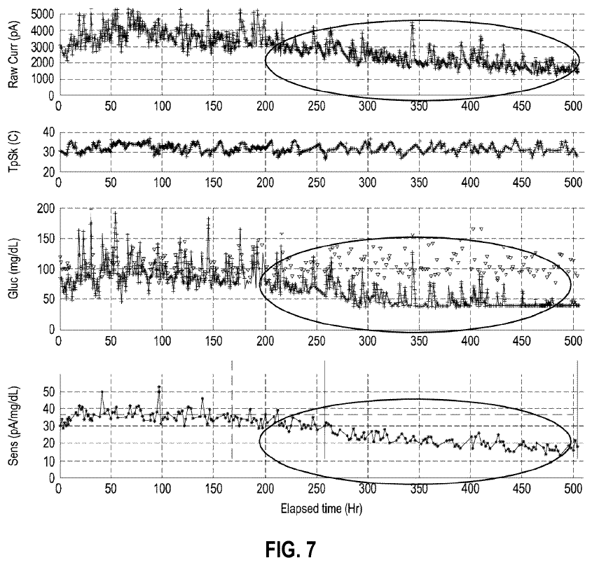

An analyte sensor comprising a representative biofilm, such as that shown in FIG. 6, was tested for glucose signal measurements over time. As shown in FIG. 7, the sensor signal showed late signal attenuation. As used herein, the term “late signal attenuation,” and grammatical variants thereof, refers to dynamic signal reduction or sudden signal reduction in sensitivity, indicating potential loss in analyte sensor functionality of accuracy. The term “late” with reference to late signal attenuation refers to any time or time period after which an analyte sensor has been positioned and functioning properly (e.g., obtaining accurate analyte measurements). As shown in FIG. 7, the x-axis represents elapsed time in hours (Hr), and the y-axis shows various data associated with a single analyte sensor. The “Raw Curr” represents the raw data (also termed “counts”) received from the operational amplifier of the analyte sensor, having units in picoamperes (pA). The “TpSk” represents the temperature readout of the analyte sensor, having units in Celsius (C); because the temperature is stable, the late signal attenuation discussed herein is not applicable. The “Glue” represents glucose measurements derived from the raw data counts, having units in milligrams per deciliter (mg/dL). The “Sens” represents the sensitivity of the analyte sensor determined using reference blood glucose measurements, having units in picoamperes per milligram per deciliter (pA/mg/dL).

As shown in FIG. 7, the raw current, glucose measurement, and sensitivity measurement each demonstrated late signal attenuation when the glucose sensor tail is coated with a biofilm and lacks any antimicrobial quality, such as roughly at about 200 hours in this particular example. Notably, the glucose sensor results shown in FIG. 7 can not be identical to those of other analyte sensors, where such trends of accuracy reduction can be characterized by reduced or increased dynamics, cyclic, or inconsistent in nature, for example.

### Comparative Example 2: Incorporation of Silver-Based Antimicrobial Quality into an Analyte Sensor Membrane

In this example from U.S. application Ser. No. 17/052,705, various silver-based antimicrobial compounds were evaluated for their effect on sensor performance when incorporated into a membrane composition. The antimicrobial compounds selected included silver carbonate (Ag₂CO₃), silver chloride (AgCl), silver iodide (AgI), and silver nanoparticles (Ag) and each were e-beam sterilized. The silver-based antimicrobial compound was intermixed with a membrane composition, as described in one or more embodiments of the present disclosure, and compared to controls. The antimicrobial compounds were intermixed at 50% by weight of Formula 1 polymer (see above), which was crosslinked with Gly3 and additionally included PDMS-DGE leveling agent.

Sensor evaluation was determined by testing the current performance of each of the silver-containing sensor membranes as compared to a control over time (in duplicate). Amperometry was run on custom built equipment with independent p-stats for each channel and data acquisition was performed. The sensors were evaluated in PBS at 37° C. An initial multistep calibration was performed from zero (0) to 30 millimoles (mmol) of glucose and, thereafter, the concentration was held at 30 mmol for the remainder of the evaluation.

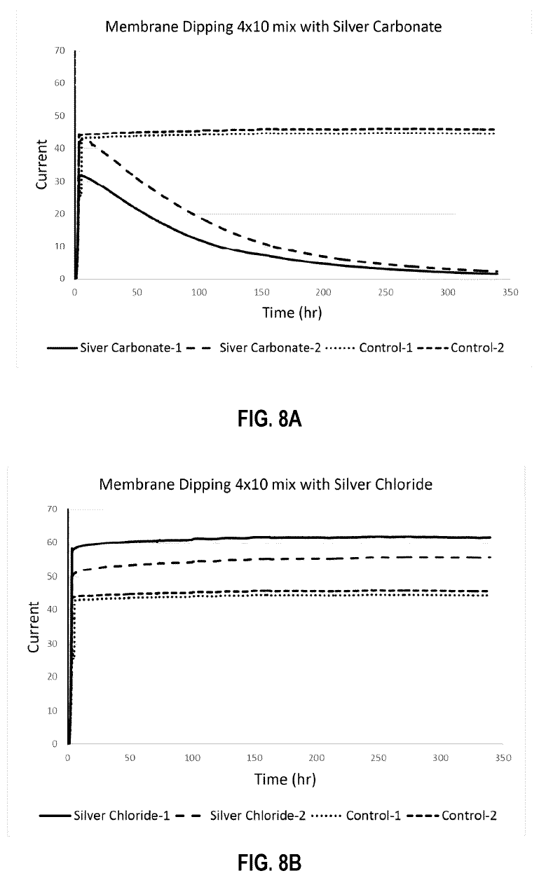

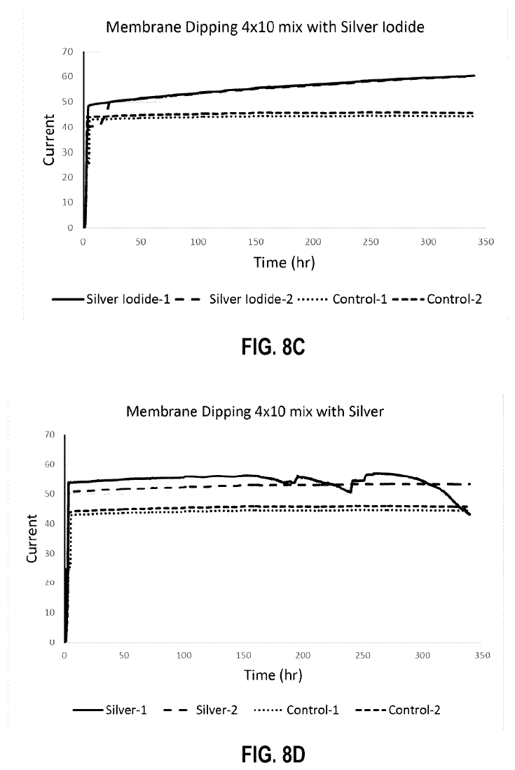

FIG. 8A shows a plot of the current response for a sensor coated with a Ag₂CO₃-containing membrane polymer against a control (duplicate measurements); FIG. 8B shows a plot of the current response for a sensor coated with a AgCl-containing membrane polymer against a control (duplicate measurements); FIG. 8C shows a plot of the current response for a sensor coated with a AgI-containing membrane polymer against a control (duplicate measurements); and FIG. 8D shows a plot of the current response for a sensor coated with a Ag-containing (nanoparticles) membrane polymer against a control (duplicate measurements). Each current response was measured in nanoamperes (nA).

As shown in FIG. 8A, the antimicrobial compound silver carbonate intermixed in the sensor membranes of the composition described in this Example resulted in relatively poor stability performance, dropping in current substantially within less than 50 hours.

FIG. 8B shows that silver chloride antimicrobial compounds incorporated into the glucose analyte sensor and membrane composition of this Example (and extrapolated to others described herein) provided steady signal performance and thus no or very minimal interference with the operation of the sensor and/or membrane. Similarly, only minimal signal drift was observed in FIG. 8C with silver iodide incorporation, and such signal drift can be corrected by incorporating other compounds or adjusting specific concentrations, for example. FIG. 8D also demonstrates that the inclusion of silver nanoparticles into the membrane composition of this Example (and extrapolated to others described herein) resulted in relatively steady signal performance. It is noted that Silver-1 (Duplicate 1 in FIG. 8D) is thought to have encountered an electrical or other flaw during testing, resulting in the sporadic signal performance not observed in Silver-2. Accordingly, for the membrane formulation of Comparative Example 2 (and extrapolated to others described herein), silver chloride, silver iodide, and silver nanoparticles show particular suitability.

### Comparative Example 3: In Vivo Testing of Silver-Based Antimicrobial Quality

In this example from U.S. application Ser. No. 17/052,705, sensors membranes comprising the silver-based antimicrobial compounds of Comparative Example 2 (tested in quadruplicate for each of the sensors in Comparative Example 2) and the Control of Comparative Example 2 were evaluated in vivo against a variety of gram-positive bacteria strains associated with common skin flora and wound infection, and which were nosocomial-derived using a modified Kirby-Bauer disk diffusion assay for antibiotic susceptibility testing. The bacteria used for evaluation were Staphylococcus aureus (MRSA) (ATCC #33591), Staphylococcus epidermidis (ATCC #12228), Enterococcus faecalis (ATCC #4082), and Streptococcus pyogenes (ATCC #19615). Each were cultured on Tryptic Soy Agar, except for S. pyogenes, which were cultured on Blood Agar. The zone of inhibition measurements are shown in Table 2 below.

TABLE 2
Zone of Inhibition Measurements

| | | Diameter of the Zone Including | |
|---|---|---|---|
| | | Test Article (mm) | |
| Organism | Sample ID | Maximum Distance | Minimum Distance |
| S. aureus | Ag₂CO₃-1 | 4.72 | 2.10 |
| (MRSA) | Ag₂CO₃-2 | 4.33 | 1.94 |
| | Ag₂CO₃-3 | 3.97 | 1.08 |
| | Ag₂CO₃-4 | 3.58 | 0.99 |
| | Control | No Zone | No Zone |
| S. epidermidis | AgCl-1 | 6.50 | 4.73 |
| | AgCl-2 | 5.78 | 4.15 |
| | AgCl-3 | 5.26 | 2.45 |
| | AgCl-4 | 4.87 | 2.19 |
| | Control | No Zone | No Zone |
| E. faecalis | AgI-1 | 4.45 | 2.48 |
| | AgI-2 | 3.98 | 1.57 |
| | AgI-3 | 3.28 | 0.93 |
| | AgI-4 | 3.93 | 1.71 |
| | Control | No Zone | No Zone |
| S. pyogenes | Ag-1 | 5.54 | 3.43 |
| | Ag-2 | 4.40 | 2.19 |
| | Ag-3 | 3.28 | 0.95 |
| | Ag-4 | 3.99 | 1.88 |
| | Control | No Zone | No Zone |

Accordingly, each of the silver-based antimicrobial compounds from Comparative Example 1 (i.e., silver carbonate (Ag₂CO₃), silver chloride (AgCl), silver iodide (AgI), and silver nanoparticles (Ag)) demonstrate antimicrobial properties, as defined herein.

### Comparative Example 4: Incorporation of Silver-Iodide Antimicrobial Quality into an Analyte Sensor Membrane

In this example from U.S. application Ser. No. 17/052,705, silver-iodide antimicrobial compound concentrations were further evaluated for their effect on sensor performance when incorporated into a membrane composition. The silver-based antimicrobial compound was intermixed with a membrane composition, as described in one or more embodiments of the present disclosure. The antimicrobial compounds were intermixed at 40%, 30%, 20%, 10%, and 5% by weight of Formula 1 polymer (see above), which was crosslinked with Gly3 and additionally included PDMS-DGE leveling agent.

Sensor evaluation was determined by testing the current performance of each of the silver-iodide sensor membranes as compared to a control over time (in various replicates), as described in Comparative Example 2 above.

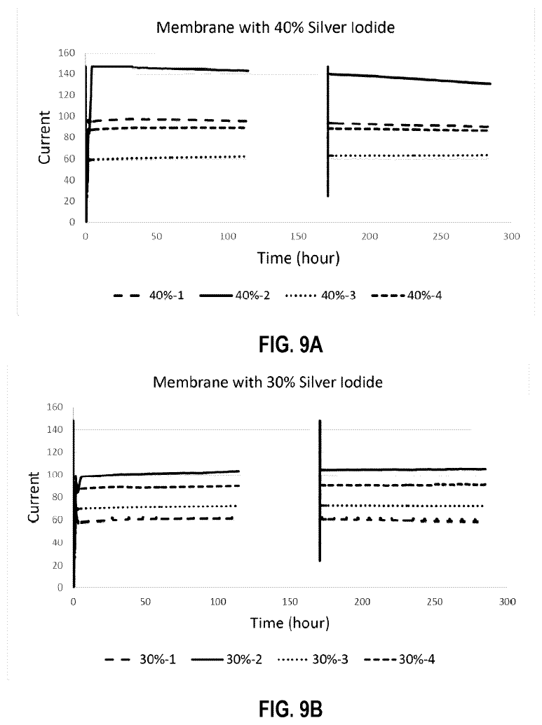

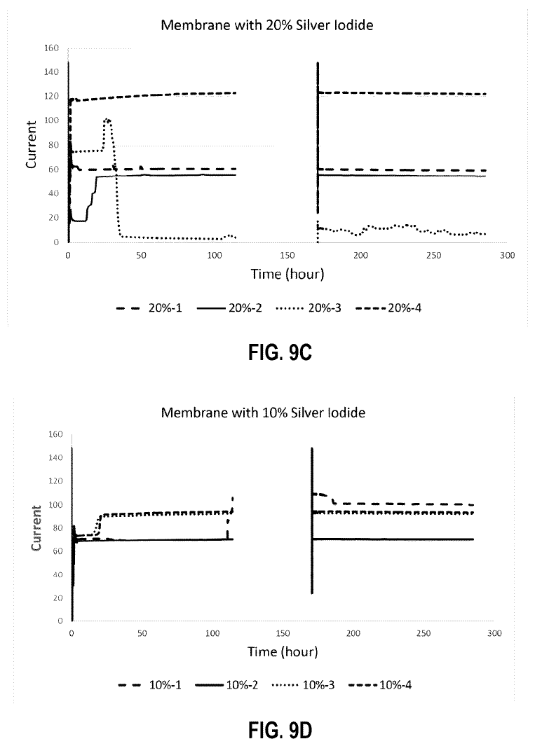

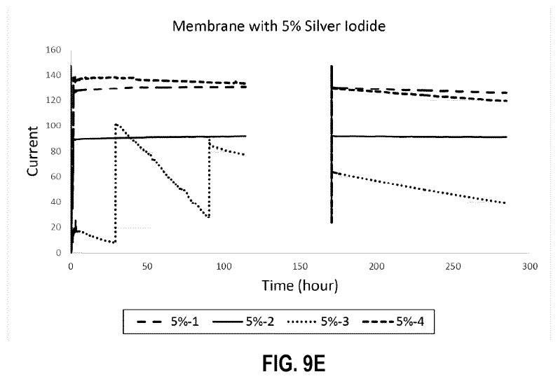

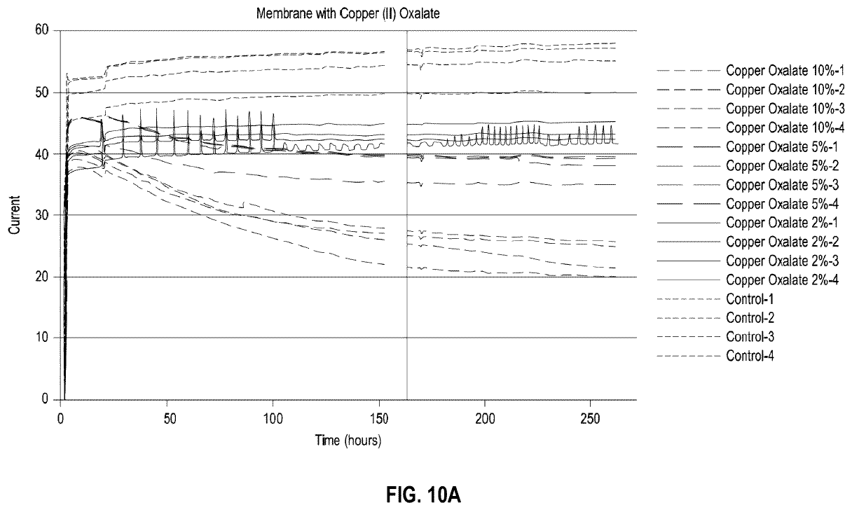

FIG. 9A shows a plot of the current response for a sensor coated with a 40% AgI-containing membrane polymer (quadruplicate measurements); FIG. 9B shows a plot of the current response for a sensor coated with a 30% AgI-containing membrane polymer (quadruplicate measurements); FIG. 9C shows a plot of the current response for a sensor coated with a 20% AgI-containing membrane polymer (quadruplicate measurements); FIG. 9D shows a plot of the current response for a sensor coated with a 10% AgI-containing membrane polymer (quadruplicate measurements); and FIG. 9E shows a plot of the current response for a sensor coated with a 5% AgI-containing membrane polymer (quadruplicate measurements). Each current response was measured in nanoamperes (nA). The break in data of FIGS. 9A-9E merely represents a time duration in which data was not received from the testing equipment.

As shown in FIGS. 9A and 9B, the 40% and 30% AgI-containing membrane polymers demonstrate a comparatively stable current signal and less signal drift.

### Comparative Example 5: Incorporation of Copper-Based Antimicrobial Quality into Membrane of Analyte Sensor

In this example from U.S. application Ser. No. 17/052,705, various copper-based antimicrobial compounds were evaluated for their effect on sensor performance when incorporated into a membrane composition. The antimicrobial compounds selected included Copper (II) Oxalate (CuC₂O₄), copper oxide (CuO), copper iodide (CuI), copper metal (Cu), copper (II) chloride (CuCl₂) and copper metal blended with silver metal (Cu/Ag) (ratio of 1:1 by weight of Cu and Ag). The antimicrobial compound was intermixed with a membrane composition, as described in one or more embodiments of the present disclosure, and compared to a Control. The antimicrobial compounds were intermixed with various loadings of the Formula 1 polymer (see above) (i.e., 10%, 5%, or 2%) by weight of Formula 1 polymer, which was crosslinked with Gly3 and additionally included PDMS-DGE leveling agent, in a syringe.

Sensor evaluation was determined by testing the current performance of each of the copper-containing sensor membranes as compared to a control over time (in various replicates), as described in Comparative Example 2 above.

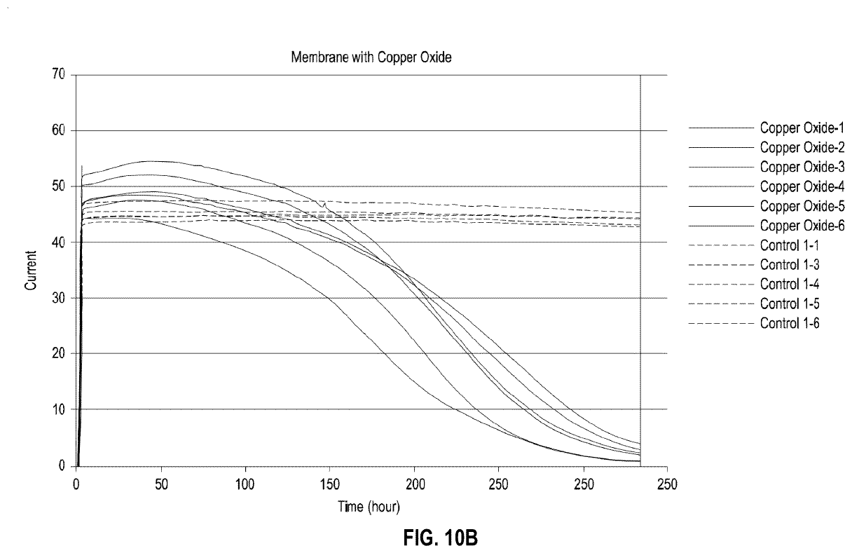

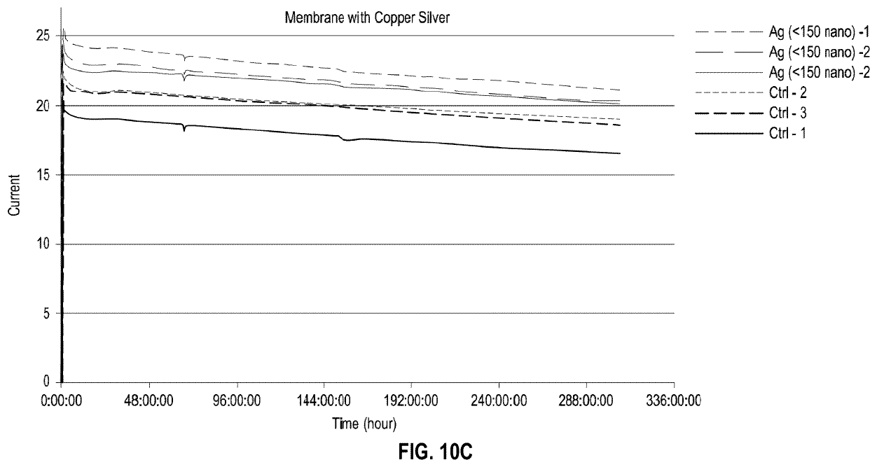

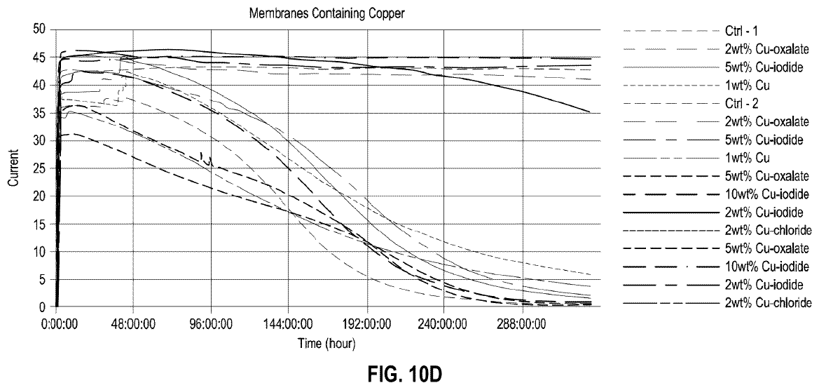

FIG. 10A shows a plot of the current response for a sensor coated with 10%, 5%, and 2% CuC₂O₄-containing membrane polymers against a control (quadruplicate measurements); FIG. 10B shows a plot of the current response for a sensor coated with 2% CuO-containing membrane polymer against a control (sextuplicate measurements); FIG. 10C shows a plot of the current response for a sensor coated with 2% Cu-containing membrane polymer (triplicate measurements), having Cu particles of less than 150 μm, against a control (triplicate measurements); and FIG. 10D shows a plot of the current response for a sensor coated with 1% Cu-containing membrane polymer (duplicate measurements), 5% or 2% CuC₂O₄-containing membrane polymers (duplicate measurements), 10%, 5%, or 2% CuI-containing membrane polymers (duplicate measurements), and 2% CuCl₂-containing membrane polymers (duplicate measurements) against a control (duplicate measurements). Each current response was measured in nanoamperes (nA).

As shown in FIGS. 10A-10D, the various copper-based antimicrobial compounds intermixed in the sensor membranes of the composition described in this Example (and extrapolated to others described herein) can be effective at providing antimicrobial qualities and good sensor performance at relatively low loading levels. At high Cu loading levels, sensor performance decreased with time. Moreover, only certain copper-based compounds appear compatible with the membrane composition of this Example (and extrapolated to others described herein) as compared to others. For example, copper (II) oxalate and copper (I) iodide appears particularly compatible.

### Example 6: Preparation of Test Sensor with 35% Weight Percent of Silver Iodide

A 10Q5 solution was prepared by mixing 140 mg/mL of 10Q5 polymer in 80:20 (volume by volume) of EtOH: 10 mM 4-(2-hydroxyethyl)-1-piperazineethanesulfonic acid (HEPES) buffer at a pH of 8. A polydimethylsiloxane (PDMS) solution was prepared by mixing 100 mg/mL of diaminopropyl polydimethylsiloxane in ethanol. A glycerol triglycidyl ether (Gly3) solution was prepared by mixing 35 mg/mL Gly3 in 80:20 (volume by volume) EtOH: 10 mM of HEPES at a pH of 8.0. The volumetric ratio of the solutions was 4 parts 10Q5 solution to 0.0132 parts of the PDMS solution to 1 part of the Gly3 solution. The silver iodide was subjected to sieving using a <53 μm sieve.

10 test sensors with the black (carbon only) side up were individually wiped with an ethanol dampened chemical wipe. The 10Q5 solution was mixed with the PDMS solution in a vial using a roller for 30 minutes to produce a mixture of 10Q5 and PDMS solutions. The Gly3 solution was mixed with 35% by weight of silver iodide (<53 μm) and with the mixture of 10Q5 and PDMS solutions to form a four-component solution. 252 μL of the four-component solution was pipetted onto each test sensor with the particles kept in suspension by occasionally stirring with the pipette tip. The test sensors were allowed to dry for 1 hour at room temperature. The test sensors were then packaged in lot labeled sterilization pouches and cured at 25° C. at 60% relative humidity for 24 hours. After curing, the test sensors were baked at 56° C. for 48 hours.

### Example 7: Minimal Essential Media Elution Test

A minimal essential media elution test was used to determine the cytotoxicity of extractable substances. An extract of the test sensor (the test article) was added to cell monolayers and incubated. The cell monolayers were examined and scored based on the degree of cellular destruction. The U.S. Pharmocopeia & National Formulary states that a test article meets its requirements and receives a passing score (PASS) if the reactivity grade is not greater than a grade of 2 (or mild reactivity). The ANSI/AAMI/ISO 10993-5 standard states that the achievement of a numerical grade of greater than 2 is considered a cytotoxic effect, or a failing score (FAIL).

The amount of test material used for extraction was based on ANSI/AAMI/ISO and USP surface or weight requirements. Test articles and controls were extracted in 1x glucose-free minimal essential media for 24-25 hours at 37±1° C. with agitation. Extracts were decanted and supplemented with 5% bovine serum post extraction. Multiple well cell culture plates were seeded with a verified quantity of industry standard L-929 cells (ATCC CCL-1) and incubated until approximately 80% confluent. The test extracts were held at room temperature for less than 4 hours before testing. The extract fluids were not filtered, centrifuged, or manipulated in any way following the extraction process. The test extracts were added to the cell monolayers in triplicate. The cells were incubated at 37±1° C. with 5±1% CO₂ for 48±3 hours. The pre and post extracted appearance of test articles and controls is described in Table 3.

TABLE 3
Pre and Post Extract Appearance.

| Substance | Stage | Appearance |
|---|---|---|
| Test Article | Pre extract | Clear with no particulates present |
| Test Article | Post extract | Clear with no particulates present - |
| | | Loss of color noted. |
| Control | Pre extract | Clear with no particulates present |
| Control | Post extract | Clear with no particulates present - |
| | | No color change noted. |

Cell monolayers were examined microscopically. The cells were scored as to the degree of discernable morphological cytotoxicity based on a relative scale of 0 to 4 shown in Table 1 3 above. The degree of discernable morphological cytotoxicity was determined by averaging the results from the three wells to provide a final cytotoxicity score.

TABLE 4
Results from Three Cell Monolayer Wells

| | | | | | | | Amount | |
|---|---|---|---|---|---|---|---|---|
| | Test | Test | Test | | Extraction | | Tested/Extraction | |
| Substance | 1 | 2 | 3 | Average | Ratio | | Solvent Amount | Result |
| Test Article | 1 | 1 | 1 | 1 | 6 | cm²/mL | 120 cm²/20 mL | PASS |
| Negative Control | 0 | 0 | 0 | 0 | 0.2 | g/mL | 4 g/20 mL | |
| (polypropylene | | | | | | | | |
| pellets) | | | | | | | | |
| Media Control | 0 | 0 | 0 | 0 | N/A | | N/A | |
| Positive Control | 4 | 4 | 4 | 4 | 0.2 | g/mL | 4 g/20 mL | |
| (Latex Natural | | | | | | | | |
| Rubber) | | | | | | | | |

As seen from Table 4, all test method acceptance criteria were met. Thus, the test sensor did not induce cytotoxicity as measured using the minimal essential media elution test according to ISO 10993-5.

### Example 8: Sensitization Testing

In selecting a new material for human contact in medical applications, it is important to ensure that the material will not stimulate the immune system to produce an allergic reaction. The reaction is generally due to substances that can leach out of a material. Therefore, the test provides for the use Freund's Complete Adjuvant (FCA) and sodium lauryl sulfate (SLS) which tend to enhance the potential of weak sensitizing agents. Testing was conducted in accordance with ISO 10993-10:2010 Biological Evaluation of Medical Devices, Part 10-Tests for Irritation and Skin Sensitization and ISO 10993-12:2012 Biological Evaluation of Medical Devices, Part 12-Sample Preparation and Reference Materials.

The black and amber test sensor (“the test article”) was prepared as indicated in Table 5.

TABLE 5
Test Article Preparation for Skin Sensitization Test

| | Test | Vehicle | Number of |
|---|---|---|---|
| | Article | Amount | Test Article |
| Test Phase and Vehicle | Area (cm²) | (mL) | Devices Used |
| First Induction Injection | 48.0 | 8.0 | 4 |
| Normal Saline (NS) | | | |
| First Induction Injection | 48.0 | 8.0 | 4 |
| Sesame Oil (SO) | | | |
| Second Induction Patch NS | 60.0 | 10.0 | 5 |
| Second Induction Patch SO | 60.0 | 10.0 | 5 |
| Challenge Patch NS | 72.0 | 12.0 | 6 |
| Challenge Patch SO | 72.0 | 12.0 | 6 |

Eleven test guinea pigs (male albino young adult guinea pigs, Hartley strain (obtained from Charles River Laboratories)) were injected with the test article and FCA, and six guinea pigs were injected with the corresponding control blank and FCA. On Day 6, the dorsal site was re-shaved and SLS in mineral oil was applied. The day after the SLS application, the test animals were topically patched with the appropriate test extract and the control animals were patched with the corresponding control blank. The patches were removed after 48±2 hours of exposure. Following an approximate two-week rest period, the animals were topically patched with the appropriate test extract and corresponding control blank. The patches were removed after 24±2 and 48±2 hours after patch removal. Each animal was assessed for a sensitization response based upon the dermal scores. The test results were based upon the percentage of animals exhibiting a sensitization response.

The following day (24±2 hours) after challenge exposure, the patches were removed and the site was wiped gently with a 70% isopropyl alcohol soaked gauze sponge prior to each scoring period. The challenge sites were observed for irritation and sensitization reaction, as indicated by erythema and edema. Daily challenge observation scores were recorded 24±2 and 48±2 hours after patch removal in accordance with the classification system for skin reactions in Table 6. Daily animal health observations were recorded throughout the study period.

TABLE 6
Dermal Observation Scoring (Magnusson and Kligman scale)

| Patch test reaction | Grading scale |
|---|---|
| No visible change - no erythema and edema² | 0 |
| Discrete or patchy erythema | 1 |
| Moderate and confluent erythema | 2 |
| Intense erythema and/or swelling | 3 |

²Erythema is defined as redness and edema is defined as swelling at the challenge site.

Grades of ‘1’ or greater in the test group generally indicate sensitization, provided grades less than ‘1’ are observed on the control animals. If grades of ‘1’ or greater are noted on control animals, then the reactions of the test animals which exceed the most severe control reaction are presumed to be due to sensitization. The outcome of the test was presented as the frequency of positive challenge results in test and control animals.

Analysis and Conclusion: None of the negative control animals challenged with the control vehicles were observed with a sensitization response greater than ‘0’. None of the animals challenged with an extract from the test article were observed with a sensitization response greater than ‘0’. The normal saline extract of the test material had a sensitization response of ‘0’ under valid test conditions. The sesame oil extract of the test material had a sensitization response of ‘0’ under valid test conditions. Under the conditions of the protocol, the test article did not elicit a sensitization response. Thus, the test sensor did not cause skin irritation as measured by the Guinea pig maximization test of Magnusson and Kligman according to ISO 10993-10.

### Example 9: Hemolytic Index Testing

A hemoglobin standard was diluted with Drabkin's reagent to give solutions at concentrations of 0.80, 0.60, 0.40, 0.20, 0.10, 0.02, and 0.01 mg/mL. The solutions were allowed to stand at room temperature for a minimum of 15 minutes. The absorbance was read on a spectrophotometer at 540 nm. A standard curve was determined using linear regression with the absorbance values and the standard concentrations of hemoglobin.

The plasma hemoglobin of the blood was determined, and based on the total hemoglobin present, the blood was diluted to 10±1 mg/mL in phosphate buffered saline (PBS). The hemoglobin concentration was determined from the standard curve and then multiplied by a factor of 16 to account for the dilution.

A test sensor (“test article”) was prepared according to Table 7.

TABLE 7
Test Article Preparation for Hemolysis Analysis

| | | Test Article/ | | |
|---|---|---|---|---|
| Extract | Extraction | Extraction | | |
| Solvent | Ratio | Solvent Amount | Temperature | Time |
| PBS | 6 cm²/mL | 156 cm²/26 mL | 50 ± 2° C. | 72 ± 2 hours |

A non-homolytic negative control, a hemolytic positive control, and a PBS blank were extracted at the same time and temperature as the test articles.

The extract fluid was held at room temperature for less than 4 hours before testing. The extract fluids were not filtered, centrifuged, or manipulated in any way following the extraction process. The PBS used in testing was calcium and magnesium free. The ASTM method (ASTM F 756) was validated using human blood, which is in compliance with ISO 10993-4:2017 Biological Evaluation of Medical Devices-Part 4: Selection of Tests for Interactions with Blood. An equal amount of human blood was drawn from 3 donors and contained 0.1 M sodium citrate in a ratio of 9:1 (3.2% anticoagulant to blood). The collected blood was refrigerated until testing was performed.

TABLE 8
Pre and Post Extract Appearance.

| Substance | Stage | Appearance |
|---|---|---|
| Test Articles | Pre extract | Clear with no particulates present |
| Test Articles | Post extract | Clear with no particulates present - |
| | | No color change noted. |
| Controls | Pre extract | Clear with no particulates present |
| Controls | Post extract | Clear with no particulates present - |
| | | No color change noted. |

The test articles were incubated in glass vials by adding 7 mL of test article or control extract and 1 mL of diluted blood. Three tubes were prepared for each test article and control. The tubes were then incubated at 37+2° C. for a minimum of 3 hours. The tubes were gently inverted twice at 30 minute intervals throughout the incubation period. After incubation, the test articles and controls were centrifuged at 700-800×g for 15 minutes and 1 mL of the supernatant fluid was combined with 1 mL of Drabkin's reagent and allowed to stand at room temperature for a minimum of 15 minutes. The test articles and controls were then read at 540 nm in a spectrophotometer. The hemolytic index was determined using the following equation:

Hemolytic Index=(hemoglobin released (mg/mL))/(hemoglobin present (mg/mL))×100

Where: hemoglobin released (mg/mL)=(optical density×X coefficient+constant)×16 and hemoglobin present (mg/mL)=diluted blood 10±1 mg/mL

The corrected hemolytic index was calculated by subtracting the hemolytic index of the PBS blank solution from the hemolytic index of the test article and controls. The test article was then compared to the negative control by subtracting the hemolytic index of the negative control from the hemolytic index of the test article. The hemolytic index and associated grade are shown in Table 9.

TABLE 9
Hemolytic Index and Grade

| Hemolytic Index | Hemolytic Grade |
|---|---|
| 0-2 | Non-Hemolytic |
| 2-5 | Slightly Hemolytic |
| >5 | Hemolytic |

As shown in Table 10, the difference between the hemolytic indexes of the test article and the negative control equals 0.00 percent. This places the test article in the non-hemolytic range according to the grade outlined in Table 9. Thus, the test sensor is non-hemolytic according to ISO 10993-4 and ASTM F 756 (which provides a standard practice for assessment of hemolytic properties of materials).

TABLE 10
Results of Hemolytic Testing

| | | | | Average | Corrected |
|---|---|---|---|---|---|
| Test | | Average | | Hemolytic | Hemolytic |
| Article/ | Optical | Optical | Hemolytic | Index (% | Index (% |
| Control | Density | Density | Index | Hemolysis) | Hemolysis) |
| Test | 0.002 | 0.003 | 0.804 | 1.05 | 0.25 |
| Article 1 | | | | | |
| Test | 0.003 | | 10.53 | | |
| Article 2 | | | | | |
| Test | 0.004 | | 1.302 | | |
| Article 3 | | | | | |
| Negative | 0.008 | 0.005 | 2.299 | 1.47 | 0.66 |
| Control 1 | | | | | |
| Negative | 0.004 | | 1.302 | | |
| Control 2 | | | | | |
| Negative | 0.002 | | 0.804 | | |
| Control 3 | | | | | |
| Positive | 0.382 | 0.394 | 95.514 | 98.4 | 97.6 |
| Control 1 | | | | | |
| Positive | 0.416 | | 103.988 | | |
| Control 2 | | | | | |
| Positive | 0.383 | | 95.763 | | |
| Control 3 | | | | | |
| PBS Blank 1 | 0.002 | 0.002 | 0.804 | 0.80 | N/A |
| PBS Blank 2 | 0.001 | | 0.555 | | |
| PBS Blank 3 | 0.003 | | 1.053 | | |

### Example 10: Intracutaneous Irritation Test

The purpose of this test was to determine if any chemicals that can leach out or be extracted from the test sensor (“the test article”) were capable of causing local irritation to the dermal tissue of rabbits. Testing was conducted in accordance with ISO 10993-10:2010 Biological Evaluation of Medical Devices, Part 10-Tests for Irritation and Skin Sensitization.

The test article was prepared as indicated in Table 11. The extraction mixtures and corresponding control blanks were incubated for 72+2 hours at 50+2° C. At the start of the extraction, the solutions appeared clear and free of particulates. The extracts were agitated during the course of the extraction period. At the end of the extraction period, the vessels were shaken well and the liquid asepticially decanted into a sterile vessel. The extracts were not further manipulated prior to use. The extracts were maintained at room temperature and used within 24 hours of preparation.

TABLE 11
Test Article Preparation for Intracutaneous Irritation Test.

| | | Vehicle | Number of Test |
|---|---|---|---|
| | Test Article | Amount | Article Devices |
| Extract Vehicle | Area (cm²) | (mL) | Used per Vessel |
| 0.9% Normal Saline | 72.0 | 12.0 | 6 |
| (NS) | | | |
| Sesame Oil (SO) | 72.0 | 12.0 | 6 |

All animals used for this study were female, young adult albino rabbits, New Zealand White strain (obtained from Robinson Services, Inc.). Each animal was weighed and the weight recorded prior to test injection. The fur of the animals was clipped on both sides of the spinal column to expose a sufficient area for injection. Each rabbit (3 total) received five sequential 0.2 mL intracutaneous injections along either side of the dorsal mid-line, with the test article solution on one side and the concurrent vehicle control on the other. The irritation reaction of the test article solutions was compared to vehicle controls and recorded over a 72 hour period according to the standard ISO irritation scoring system. The dermal observation scoring is shown in Table 12.

TABLE 12
Dermal Observation Scoring

| | |
|---|---|
| | Numerical |
| Reaction | Grading |

Erythema and eschar formation

| | |
|---|---|
| No erythema | 0 |
| Very slight erythema (barely perceptible) | 1 |
| Well-defined erythema | 2 |
| Moderate erythema | 3 |
| Severe erythema (beet-redness) to eschar formation preventing | 4 |
| grading of erythema | |

Oedema formation

| | |
|---|---|
| No oedema | 0 |
| Very slight oedema (barely perceptible) | 1 |
| Well-defined oedema (edges of area well-defined by definite | 2 |
| raising) | |
| Moderate erythema (raised approximately 1 mm) | 3 |
| Severe oedema (raised more than 1 mm and extending beyond | 4 |
| exposure area) | |
| Maximal possible score for irritation | 8 |

Other adverse changes at the injection site to be recorded and reported.

The animals were observed daily for abnormal clinical signs. The appearance of each injection site was noted immediately post injection and at 24±2, 48±2, and 72±2 hours.

The tissue reactions were rated for gross evidence of erythema and edema. The intradermal injection of SO frequently elicits an inflammatory response. SO erythema scores≤2 are considered normal. A well-defined positive response is characterized by a score equal to or greater than 2.

After the 72±2 hour observation period, all erythema grades plus edema grades at 24±2, 48±2, and 72±2 hours were totaled separately for each test sample or control for each individual animal. To calculate the score of a test sample or control on each individual animal, each of the totals was divided by 15 (three scoring time points x five test or control sample injection sites). To determine the overall mean score for each test sample and each corresponding control, the scores were added for the three animals and divided by three. The final test score was obtained by subtracting the score of the control from the test sample score.

According to ISO 10993:10 test criteria, if the difference between the average scores for the extract of the test article and the vehicle control is less than or equal to 1.0, the test article is considered to have met the requirements of the test.

Analysis and Conclusion: The difference in the mean test and control scores of the extract dermal observations were 1.0 or less, indicating that the requirements of the ISO Intracutaneous Reactivity Test have been met by the test article. Thus, the test sensor does not cause skin irritation as measured by the intracutaneous reactivity test according to ISO 10993-10.

### Example 11: Bacterial Reverse Mutation Assay

The bacterial reverse mutation assay (Ames test) was used to determine the potential mutagenic activity of a solid test article extract by exposing a large number of the test organisms to the extract fluid in agar plates. The agar plates were monitored for growth of revertants (organisms mutating to the wild type) which were counted and used to estimate the mutagenic potential of the test article. Testing was conducted in accordance with ISO 10993-3:2014 Biological Evaluation of Medical Devices, Part 3-Tests for Genotoxicity, Carcinogenicity and Reproductive Toxity and OECD Guidelines for Testing of Chemicals, Section 4, Test No. 471: Bacterial Reverse Mutation Test (OECD 471).

The Ames test employs several histidine auxotrophic (His-) strains of Salmonella typhimurium, which require the amino acid histidine for growth, ana tryptophan auxotrophic (Trp-) strain of Escherichia coli, which requires tryptophan for growth. The test detects mutations which cause the bacterial stranis to revert to histidine or tryptophan independent (His+ or Trp+) bacteria. These revertants are detected by their ability to grow in the absence of an external source of histidine or tryptophan, respectively. The assay uses S. typhimurium tester strains TA97a, TA98, TA100, and TA1535, and E. coli test strain WP2, which were selected to detect various types of mutagens. The S-9 activation system is designed to simulate mammalian liver enzyme systems and is used to detect substances which undergo metabolic activation from non-mutagenic forms.

Test sensors (“test articles”) were prepared as shown in Table 13. The amount of test material was based on ISO extraction ratios. An aliquot of the solvents used were included and incubated in parallel with the test articles to serve as solvent controls (negative controls).

TABLE 13
Test Article Preparation

| | | Test Article/ | Extraction | Extraction |
|---|---|---|---|---|
| | Extraction | Extraction | Temperature | Time (with |
| Extract Vehicle | Ratio | solvent amount | (with agitation) | agitation) |
| 0.9% NaCl (saline) | 6 cm²/mL | 60 cm²/10 mL | 50 ± 2° C. | 72 ± 2 hours |
| PEG 400 adjusted to a physiological | 6 cm²/mL | 60 cm²/10 mL | 50 ± 2° C. | 72 ± 2 hours |
| osmotic pressure | | | | |

The extract fluids were not filtered, centrifuged, or manipulated in any way following the extraction process. The appearance of the extract fluids are shown in Table 14.

TABLE 14
Pre and Post Extract Appearance.

| Substance | Stage | Appearance |
|---|---|---|
| Test Articles | Pre extract | Clear with no particulates present |
| Test Articles | Post extract | Clear with no particulates present - |
| | | No color change noted. |
| Controls | Pre extract | Clear with no particulates present |
| Controls | Post extract | Clear with no particulates present - |
| | | No color change noted. |

Frozen working cultures of each strain were used to inoculate nutrient broth for testing. The cultures were incubated for 5.0-5.5 hours on an orbital shaker at approximately 100 rpm. The titers of the cultures were determined and had concentrations of approximately 108 CFU/mL or higher. The cultures were verified for presence of appropriate strain phenotype characteristics.

The S-9 activation system was used to screen for the presence of mutagents from byproducts of the test article. Rat liver S-9 homogenate (obtained from Molecular Toxicology, Inc.), kept frozen at ≤−60° C., were plated to contain approximately 20 μL rat liver S-9 per plate. When working with soft agar the plates did not exceed 47° C.

Aliquots of top agar were melted and maintained at 45±2° C. For test strains TA97a, TA98, TA100, and TA1535, each 100 mL aliquot of top agar was fortified with 5-10 mL of 0.5 mM biotin and 0.5 mM histidine solution prior to use. For test strain WP2, each 100 mL aliquot of top agar was fortified with 5-10 mL of 0.5 mM L-tryptophan solution.

Each test article extract and solvent control was tested both with and without S-9 metabolic activation. The S-9 specific chemical controls (2-aminofluorene and 2-aminoanthracene) were also tested with S-9 metabolic activation only. Strain specific non-metabolic chemical controls were also included (sodium azide, methyl methanesulfonate, and 4-nitro-O-phenylene-diamine). The non-metabolic chemical controls were tested without S-9 activation only.

Sterile 13×100 mm test tubes were transferred to a waterbath held at 45±2° C. Two mL aliquots of top agar were transferred to each test tube. Three replicates for each test article or control were prepared. The test organisms and materials were added as specified in Table 15.

TABLE 15
Test Organism and Material Addition

| Identification | Added/Replicate |
|---|---|
| Solvent controls | 2 mL top agar, 100 μL test organism, and |
| | 100 μL of each solvent control |
| Test article extracts | 2 mL top agar, 100 μL test organism, and |
| | 100 μL of test article extract |
| Chemical controls | 2 mL top agar, 100 μL test organism, and |
| | 100 μL of the specified chemical |

Each replicate requiring S-9 metabolic activation had 0.5 mL of the prepared S-9 mix added. The replicates were vortexed, poured onto MGPA plates, swirled to form an even layer, and allowed to solidify. The plates were incubated for growth at 37+2° C. for 48-72 hours.

The test articles were also analyzed using the spot method on plates with and without the S-9 activation system. Two mL aliquots of the top agar mixture and 0.1 mL of the appropriate test organism was added to minimal glucose agar plates. The plates were allowed to harden then 10 μL of the test article extract was added as a spot on the surface of the plate. The plates were incubated for growth of the organisms at 37±2° C. for 48-72 hours.

The following chemical controls were used (with the concentrations listed as the amount added per plate): 1.5 μg sodium azide, 100 μg methyl methanesulfonate, 20 μg 4-nitro-O-phenylenediamine, 20 μg 2-aminofluorene, and 7 μg 2-aminoanthracene. The chemical controls were tested using the plate incorporation method only.

The criteria for acceptance of the test and criteria for determination of a mutagen are provided as follows:
1) Tested strains for phenotype verification and achieved the appropriate responses.
2) All chemical controls included in the test gave the appropriate responses.
3) The reversion rates for each tester strain within the historical ranges as outlined in the standard test protocol. Historical data are constantly changing, as new data from acceptable tests are created. Therefore, reversion rates can differ slightly.

Criteria for a mutagen: a reversion rate greater than 200% of the solvent control in strains TA97 and TA100. A reversion rate greater than 300% of solvent control in strains TA98, TA1535, and WP2.

Criteria for a non-mutagen: a reversion rate less than or equal to 200% of the solvent control in strains TA97 and TA100. A reversion rate less than or equal to 300% of solvent control in strains TA98, TA1535, and WP2.

The results were calculated using a validated computer program. All results greater than 300 colony forming units (CFU) are considered estimates. The test article extracts did not produce a two-fold or three-fold increase in the number of revertants in any of the 5 tester strains. The spot tests showed no zone of increased reversion or of toxicity. In summary, the extracts tested against the five strains did not meet the criteria for a potential mutagen. Thus, the test sensor was not a potential mutagen as measured by the Ames Bacterial Reverse Mutation Assay according to ISO 10993-3 and OECD 471.

### Example 12: Acute Systemic Injection Test

Animals were treated by intravenous or intraperitoneal routes to screen solutions or test article extracts for potential toxic effects as a result of a single-dose systemic injection. The animal species, number, and route of test article administration were as recommended in ISO 10993-11:2017 Biological Evaluation of Medical Devices-Part 11: Tests for Systemic Toxicity. For the safety evaluation of the test article, mice were injected systemically with extracts of the test sensor (“the test article”) in standard solutions (normal saline and sesame oil). The animals were observed for signs of toxicity immediately after injection and at 4, 24, 48, and 72 hours post-injection. The requirements of the test are met if none of the animals treated with the test article extract have a significantly greater adverse reaction than the animals treated with the vehicle control.

The animals used in this testing were albino, young adult, female, Swiss mice, ND4 naïve (obtained from ENVIGO). The study used 10 mice per extract vehicle (5 test, 5 control). Test articles were prepared as shown in Table 16.

TABLE 16
Test Article Record for Systemic Toxicity Test

| | | Vehicle | Number of Test |
|---|---|---|---|
| | Test Article | Amount | Article Devices |
| Extract Vehicle | Area (cm²) | (mL) | Used per Vessel |
| 0.9% Normal Saline (NS) | 60.0 | 10.0 | 5 |
| Sesame Oil (SO) | 60.0 | 10.0 | 5 |

The extraction mixtures and corresponding control blanks were incubated for 72±2 hours at 50±2° C. At the start of extraction, the solutions appeared clear and free of particulates. The extracts were agitated during the course of the extraction period. At the end of the extraction period, the vessels were shaken well and the liquid aseptically decanted into a sterile vessel. Fragments in extracts for intravenous dosing were allowed to settle prior to dosing. The extracts were not further manipulated prior to use. The extracts were maintained at room temperature and used within 24 hours of preparation.

Groups of five animals were injected with either the test article extract or the corresponding control vehicle as indicated in Table 17.

TABLE 17
Routes, Dosages, and Injection Rates

| Extract or Control | Route | Dose | Injection Rate |
|---|---|---|---|
| NS | Intravenous | 50 mL/kg | ~0.1 mL/sec |
| SO | Intraperitoneal | 50 mL/kg | Not Applicable |

According to ISO guidelines, the test is considered negative if none of the animals injected with the test article show a significantly greater biological reaction than the animals treated with the control vehicle. Death in two or more mice or other toxic signs such as convulsions, prostration, or body weight loss greater than 10% of body weight in three or more control animals is also considered an abnormal clinical sign.

Results: None of the animals in the study were observed with abnormal clinical signs indicative of toxicity during the 72 hour test period. All were alive at the end of the 72 hour test duration and body weight changes were within acceptable parameters over the course of the study as shown in Table 18. Thus, the test sensor does not cause systemic toxicity as measured by the Acute Systemic Toxicity Test according to ISO 10993-11.

TABLE 18
Mortality, Clinical Signs, and Weight Loss Incidence

| | | | | | Animals with >10% | |
|---|---|---|---|---|---|---|
| | Fatalities | | Toxicity Clinical Signs | | Body Weight Loss | |
| Extract | Test | Control | Test | Control | Test | Control |
| NS | 0/5 | 0/5 | 0/5 | 0/5 | 0/5 | 0/5 |
| SO | 0/5 | 0/5 | 0/5 | 0/5 | 0/5 | 0/5 |

### Example 13: Intramuscular Injection Test

The purpose of this test was to determine the local effects of a test article in direct contact with living skeletal muscle tissue of a rabbit after a four week duration. Testing was conducted in accordance with ISO 10993-6:2016 Biological Evaluation of Medical Devices, Part 16-Tests for Local Effects after Implantation.

All animals used for this study were male, young adult, albino rabbits, New Zealand White strain (obtained from Robinson Services, Inc.). Each animal was weighed and the weight recorded prior to test injection. The fur of the animals was clipped on both sides of the spinal column to expose an adequate area for implantation. Loose fur was removed from the skin. Each rabbit (3 total) was anesthetized with a combination of 20 mg/kg of ketamine hydrochloride and 0.75 mg/kg of acepromazine prior to implantation. In addition, supplemental isoflurane gas anesthesia was used to maintain the surgical plane to prevent muscular movements. To prepare for surgical implantation, the back of each rabbit was scrubbed with Betadine solution and cleaned with 70% isopropyl alcohol. The animals were draped for aseptic surgical procedure.

A single incision was made in the skin along the mid-line of the animal's back. The upper layer of fascia were also incised to allow access to the paravertebral muscles. A small incision, large enough to accommodate the size of the implant material, was made in the paravertebral muscle and a hemostate was gently inserted to prepare the area for the test or control material.

Each animal had five strips of USP high density polyethylene reference standard implanted into the left paravertebral muscle, via incisions approximately 1 cm into the muscle. In a similar fashion, five test sensor (“the test article”) samples were implanted into the right paravertebral muscle.

The muscle and fascia at each implanted site was closed with an absorbable suture. The mid-line skin incision was closed with suture. Three rabbits were implanted with the test article and control article during the above procedure. The animals were then returned to their original cages and observed daily for the duration of the study. Each rabbit was given an injection of an analgesic (buprenorphine SR) on the day of implantation.

At the end of the four week period, the test paravertebral muscles were dissected away from the spine of sacrificed animals. The tissues were then placed into properly labeled tissue cups in 10% NBF. The tissue surrounding the implant was examined microscopically for the presence of hemorrhage, fluids, encapsulation, discoloration, or presence of infection. These observations were recorded. The tissue sections were processed into paraffin, cut, and stained for light microscopy. Slides were interpreted by a trained pathologist.

The final evaluation of this test was based on the histological comparison of the test article to the comparison control material. The gross observations at necropsy were used as an aid to the study pathologist to develop a correlation between gross and microscopic observations.

Results: All animals survived to the scheduled study endpoint. No abnormal clinical signs were noted for any of the animals during the course of the study. The explant observations were normal. The average of the test article site scores (10.5) minus the average of the control site scores (9.6) equals the irritant ranking score (0.9). Based on the observations of the study pathologist an irritant ranking score of 0.9 was calculated indicating the test article was a non-irritant as compared to the USP high density polyethylene reference standard. Thus, the test sensor does not cause intramuscular irritation as measured by the Intramuscular Implantation Test according to ISO 10993-6.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification. As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

What is claimed is:

1. An analyte sensor comprising:
a sensor tail substrate having a lower portion and configured for insertion into a tissue;
a first working electrode disposed on the lower portion of the sensor tail substrate;
a sensing layer disposed upon a surface of the first working electrode; and
a membrane disposed over at least the sensing layer;
wherein the membrane comprises 20% to 50% by weight of a silver salt with a particle size of ≤60 μm.

2. The analyte sensor of claim 1, wherein the silver salt is uniformly distributed within the membrane.

3. The analyte sensor of claim 1, wherein the silver salt is selected from the group consisting of silver chloride, silver-silver chloride, silver iodide, and combinations thereof.

4. The analyte sensor of claim 1, wherein the silver salt is silver iodide.

5. The analyte sensor of claim 4, wherein the silver iodide has a particle size of ≤53 μm.

6. The analyte sensor of claim 4, wherein the silver iodide has a purity of at least 99%.

7. The analyte sensor of claim 4, wherein the membrane comprises 30% to 40% by weight of silver iodide.

8. The analyte sensor of claim 4, wherein the membrane comprises 35% by weight of silver iodide.

9. The analyte sensor of claim 1, wherein the analyte sensor does not sensor does not induce cytotoxicity as measured according to ISO 10993-5.

10. The analyte sensor of claim 1, wherein the analyte sensor does not cause skin irritation or skin sensitization according to ISO 10993-10.

11. The analyte sensor of claim 1, wherein the analyte sensor does not cause intradermal irritation according to ISO 10993-10.

12. The analyte sensor of claim 1, wherein the analyte sensor does not cause systemic toxicity according to ISO 10993-11.

13. The analyte sensor of claim 1, wherein the analyte sensor is non-hemolytic according to ISO 10993-4 and ASTM F 756.

14. The analyte sensor of claim 1, wherein the analyte sensor does not cause intramuscular irritation according to ISO 10993-6.

15. The analyte sensor of claim 1, wherein the analyte sensor is not a potential mutagen according to ISO 10993-3 and OECD 471.

16. A method of inhibiting microorganism growth in an area in and around an insertion site comprising inserting an analyte sensor in a subject in need thereof comprising:
a first portion positionable above a surface of the skin;
a second portion positionable below the surface of the skin, wherein the second portion is in contact with bodily fluid and configured to measure signals indicative of analyte levels in the bodily fluid;
a first working electrode disposed on the second portion;
a sensing layer disposed upon a surface of the first working electrode; and
a membrane disposed over at least the sensing layer;
wherein the membrane comprises 20% to 50% by weight of a silver salt with a particle size of ≤60 μm, and wherein the analyte sensor does not induce cytotoxicity as measured according to ISO 10993-5.

17. The method of claim 16, wherein the silver salt is silver iodide.

18. The method of claim 17, wherein the silver iodide has a particle size of ≤53 μm.

19. The method of claim 17, wherein the silver iodide has a purity of at least 99%.

20. A method of preparing an analyte sensor overcoated with a membrane comprising:
a) mixing a silver salt having a particle size of ≤60 μm and at least one membrane polymer to form a silver salt dispersion, wherein the dispersion comprises 20% to 50% by weight of the silver salt;
b) dipping an analyte sensor into the silver salt dispersion to overcoat the analyte sensor with the silver salt dispersion;
c) curing the analyte sensor; and
d) baking the analyte sensor.

21. The method of claim 20, wherein the silver salt is silver iodide.

22. An analyte sensor comprising:
a sensor tail substrate having a lower portion and configured for insertion into a tissue;
a first working electrode disposed on the lower portion of the sensor tail substrate;
a sensing layer disposed upon a surface of the first working electrode;
a membrane disposed over at least the sensing layer; and
a non-electrochemical functional layer comprising a silver salt;
wherein the analyte sensor does not induce cytotoxicity as measured according to ISO 10993-5.

23. The analyte sensor of claim 22, wherein the silver salt is silver iodide with a particle size of ≤60 μm and a purity of at least 99%.

24. The analyte sensor of claim 22, wherein the silver salt is silver-silver chloride.
